(19) United States
(12) Patent Application Publication
(10) Pub. No.: US 2009/0176773 A1
(43) Pub. Date: Jul. 9, 2009

(54) Non-Peptidic Inhibitors of AKAP/PKA Interaction

(75) Inventors: Enno Klussmann, Berlin (DE); Walter Rosenthal, Kleinmachnow (DE); Jorg Rademann, Berlin (DE); Frank Christian, Berlin (DE); Sina Meyer, Berlin (DE)

Correspondence Address: JOYCE VON NATZMER;PEQUIGNOT + MYERS LLC, 200 Madison Avenue, Suite 1901, New York, NY, 10016, US

(73) Assignee: FORSCHUNGSVERBUND BERLIN E.V., Berlin (DE)

(21) Appl. No.: 11/914,591

(22) PCT Filed: May 18, 2006
(86) PCT No.: PCT/DE06/00897

§ 371 Date: Aug. 8, 2008

(30) Foreign Application Priority Data
May 18, 2005 (DE) 102005023505.0
Jan. 16, 2006 (DE) 102006002614.4

(51) Int. Cl. A61K 31/536; C07D 207/44; A61K 31/40; C07K 16/44; A01K 67/027; C12N 5/00; C07D 265/30; C07D 241/04; A61K 31/5375; A61K 31/495; C07D 319/12; A61K 31/357; C07D 413/06; A61K 31/421; C07D 257/06; A61K 31/41; C07D 233/96; A61K 31/4166; C07D 211/82; A61K 31/44; C07D 413/10; C07D 407/12; A61K 31/5355; A61P 3/04; A61P 3/10; A61P 9/00; A61P 11/00; A61P 9/04; A61P 25/18; A61P 11/06; A61P 29/00; A61P 31/18

(52) U.S. Cl. 514/230.5; 548/572; 514/428; 530/387.1; 800/11; 435/375; 800/9; 548/569; 514/429; 544/168; 544/400; 514/237.8; 514/252.12; 514/255.02; 544/383; 549/378; 514/452; 548/228; 514/376; 548/251; 514/381; 548/325.5; 514/386; 546/329; 514/357; 544/92; 549/366; 544/96; 514/228.8

(57) ABSTRACT

The invention relates to non-peptidic molecules which modulate, especially inhibit, the interaction of protein kinase A (PKA) and A kinase anchor proteins (AKAP) and to a host or target organism that comprises said non-peptidic compounds or recognition molecules directed to said compounds, such as e.g. antibodies or chelating agents. The invention also relates to a pharmaceutical agent, especially for use in the treatment of diseases that are associated with a disturbance of the cAMP signal path, especially insipid diabetes, hypertonia, pancreatic diabetes, duodenal ulcer, asthma, heart failure, obesity, AIDS, edema, hepatic cirrhosis, schizophrenia and others. The invention also relates to the use of the inventive molecules.

The invention relates to non-peptidic molecules which modulate, especially inhibit, the interaction of protein kinase A (PKA) and protein kinase A anchor proteins (AKAP) and to a host or target organism that comprises said non-peptidic compounds or recognition molecules directed thereto, such as antibodies or chelating agents; the invention also relates to a pharmaceutical agent, especially for the treatment of diseases associated with a disorder of the cAMP signaling pathway, especially insipid diabetes, hypertonia, pancreatic diabetes, duodenal ulcer, asthma, heart failure, obesity, AIDS, edema, hepatic cirrhosis, schizophrenia and others. The invention also relates to the use of the inventive molecules.

The ability of cells to receive signals from outside and respond to them is of fundamental importance to their survival and their function. The signals are detected by receptors and converted into a cellular response which invariably involves a chemical process.

The existence of a large number of possible signals and an even larger number of possible reactions necessitates numerous signaling pathways. One possible signaling pathway that can be followed is the cAMP-dependent signaling pathway which is initiated by activation of a G protein-coupled heptahelical transmembrane receptor by an extracellular signal, e.g. by a neurotransmitter or a hormone. The best-investigated example of such a signaling pathway is the β-adrenergic receptor system wherein G protein-coupled β-adrenergic receptors (GPCR) are activated by adrenaline or noradrenaline. β-Adrenergic receptors are differentiated into the subtypes beta₁, beta₂ and beta₃ which differ in their tissue distribution and ligand affinity.

The receptor thus activated transmits the signal to a heterotrimeric, guanosine triphosphate-binding protein (GTP-binding protein or G protein) which subsequently replaces bound GDP with GTP and is activated as well.

Following GTP binding, the trimer dissociates into an alpha-subunit and a beta-gamma-subunit. Both subunits are bound to the membrane via acylation, each one being capable of activating and inhibiting effectors of their own.

G proteins have intrinsic GTPase activity which can cleave bound GTP and re-deactivate the G protein, so that the alpha and beta-gamma-subunits re-form the trimer. Consequently, the G proteins assume the function of a molecular switch. G proteins are divided into different classes (Gₛ, Gᵢ, G_{q}, G_{olf}), the subunits of which can activate or deactivate various effectors. The above effectors include Ca²⁺ and K⁺ channels, phospholipase C-beta and adenylate cyclase (AC).

Adenylate cyclase, likewise a membranous enzyme occurring in several variants, is stimulated by Gₐₗₚₕₐₛ. It converts ATP into the second messenger cAMP. cAMP can freely diffuse into the cytosol and activate various effectors, including cyclic nucleotide-gated (CNG) cation channels opening or closing upon cAMP binding, and a family of guanine nucleotide exchange factors (GEF), the so-called EPACs (exchange proteins activated by cAMP). The latter regulate the small monomeric GTPases Rap-1 and Rap-2 which act as suppressors of Ras. Binding of cAMP to EPAC results in a conformational change exposing and activating the GEF domain. In this way, the suppressing effect is terminated, and Ras can be activated by the GEF function. Ras is involved in MAP kinase (mitogen-activated protein kinase) signaling pathways activated by a variety of signals, e.g. from cytokines, which induce proliferation or differentiation.

In addition, cAMP can influence gene expression via cAMP-responsive element-binding proteins (CREB); CREB bind to the cAMP-responsive elements (CRE) of DNA, thus acting as transcription factors.

However, the most extensively described and best-characterized effector of cAMP is the cAMP-dependent protein kinase (PKA or A kinase). In the inactive state, the protein is in the form of a heterotetramer, consisting of two regulatory subunits (R) and two catalytic subunits (C). In such a bound state, the catalytic subunits are inactive. When the intracellular cAMP concentration rises following stimulation, two cAMP molecules bind to each R subunit. As a result of the thus-triggered conformational change, the catalytic subunits are released, subsequently phosphorylating nearby target proteins on Ser and Thr residues, thereby causing conformational and thus functional changes therein as well. Recognition of the target proteins proceeds via consensus sequences.

In view of the facts that many different extracellular stimuli converge in the cAMP-PKA pathway, and that PKA is an enzyme with broad substrate specificity, the question arises in which way this wide variety of stimuli could trigger different specific cell responses.

One initial contribution to specificity in the cAMP-PKA signaling pathway is provided by R and C subunits (RIalpha, RIbeta, RIIalpha, RIIbeta and Calpha, Cbeta, Cgamma, PrKX, respectively) which, depending on the type of cell, are present in different isoforms and may undergo aggregation into functionally different PKA isoforms. In general, RI subunits have higher affinity to cAMP than RII subunits. R subunits can form both homo- and heterodimers, allowing many possible combinations with finely graded affinity.

In addition, local aspects play an important role in specificity of the signaling pathway: cAMP is produced by AC at a particular site near the G protein-coupled receptors, and free diffusion in the cytosol is restricted by phosphodiesterases (PDE) which hydrolyze cAMP to form adenosine monophosphate. In this way, an extracellular stimulus will only activate PKA tetramers situated within the cAMP area of activity locally restricted by PDE.

An A kinase anchor protein (AKAP) assumes the important function of anchoring the PKA not only within such an area of activity, but also near substrates to be phosphorylated.

AKAP proteins are a family of about 50 proteins at present, which have no significant sequence homology but similar function. AKAP proteins are defined by the feature of a binding domain for the R subunits of PKA. This conserved motif is an amphipathic alpha-helix of 14 to 18 amino acids, the hydrophobic side of which interacts with a hydrophobic pocket of PKA.

The hydrophobic pocket is formed by the N termini of dimerized R subunits. The N termini undergo aggregation in an antiparallel fashion, forming a four-helix bundle from two helix-turn-helix motifs. The alpha-helices of each subunit which are closest to the N terminus form the hydrophobic pocket required for AKAP binding, while the alpha-helices of the bundle following in C-terminal direction allow dimerization. Apart from the amino acids contributing to the alpha-helices, further N-terminal amino acid side chains are involved in interactions with AKAP proteins.

The N-terminal portion of the RI and RII subunits varies with respect to the sequence, resulting in varying AKAP binding specificity. Most AKAP proteins bind to RII subunits (K_{d} in the nanomolar range), in which case low- and high-affinity AKAP proteins are distinguished. While AKAPLbc/Ht31, AKAP79 and AKAP95, having K_{d} values between 1 and 50 nM, are included among high-affinity AKAP proteins, gravin and AKAP proteins included in the ezrin/radixin/moesin protein family are included among those having low affinity (K_{d} values in the micromolar range). In addition, there are some dual-specific AKAP proteins (D-AKAP) binding both R types. AKAP_{CE} (from Caenorhabditis elegans), AKAP82, PAP7 (peripheral-type benzodiazepine receptor (PBR) associated protein 7) and hAKAP220 are the only RI-specific AKAP proteins known to date.

Another characteristic domain of AKAP proteins is the so-called targeting domain that anchors each AKAP protein on a specific subcellular compartment in the cell.

In addition to PKA and subcellular structures, AKAP proteins can bind other signaling molecules and important enzymes in the cAMP-PKA system, such as PDE and phosphatases. The latter reverse phosphorylation reactions by hydrolysis, thus being capable of terminating the effect of a stimulus. In this way, AKAP proteins provide a framework near the substrate, on which all proteins required for initiation and termination of a signal can be gathered. With substrate phosphorylation by the AKAP-fixed PKA, the cAMP signal coming from AC, being diffuse in principle, is thus focused with regard to space and time.

AKAP proteins are centrally involved in many cellular processes. One possible classification is localization of the AKAP proteins within the cell. Thus, there are ion channel-, cytoskeleton- and mitochondria-associated AKAP proteins. Another possible way is provided by tissue-specific expression of AKAP proteins.

To demonstrate the importance of AKAP proteins to signal transduction pathways in cells, the functions of three AKAP proteins, gravin, AKAP79 and AKAP18, will be described in more detail below.

Gravin is an actin-associated AKAP; polymerized actin is an important component of the cytoskeleton. Gravin serves as multivalent scaffolding protein which, in addition to actin, binds PKA and Ca²⁺-dependent protein kinase (PKC). This complex is important in desensitization of the β₂-adrenergic receptor (see above) following stimulation by agonists. In the state of rest, the gravin complex is bound to the receptor. Activation of the receptor, e.g. by adrenaline binding, results in formation of cAMP (see above). The gravin-anchored PKA releases its catalytic subunits which phosphorylate gravin itself, which phosphorylation initially enhances binding. If stimulation by the agonist continues, gravin is also phosphorylated by PKC which is anchored as well. Such phosphorylation causes dissociation of the gravin complex from the receptor. Instead, the β-arrestin adaptor protein bound to the mouse double minute 2 (MDM2) ubiquitin ligase binds to the receptor. The MDM2-catalyzed ubiquitination characterizes the receptor for proteasomal degradation and endocytosis, by means of which the β₂-adrenergic receptor system is desensitized.

AKAP79 is an ion channel-associated AKAP involved in regulation of the synaptic plasticity in the hippocampus. Mediated by PKA, the flux of Na⁺ or Ca²⁺ induced by α-amino-3-hydroxy-5-methyl-4-isoxazolylpropionic acid (AMPA) receptors is enhanced therein. AMPA receptors have an intrinsic Ca²⁺/Na⁺ channel function. Enhancement is achieved by modulating the activity of the glutamate receptor via phosphorylation. AKAP79 assumes the function of anchoring PKA in the vicinity of the AMPA receptors. To this end, it has N-terminal basic regions which allow binding to the phosphatidylinositol-4,5-bisphosphate membrane lipid. Binding of AKAP79 to the receptor does not proceed directly; rather, it binds to membrane-associated guanylate kinase proteins (MAGUK) which in turn bind to the receptor.

AKAP18 has first been described as a membrane-associated protein 15 or 18 kDa in size, which anchors PKA on the basolateral plasma membrane of epithelial cells and in skeletal muscle cells in the vicinity of L-type Ca²⁺ channels on the plasma membrane, with AKAP18 directly interacting with the C terminus of the Ca²⁺ channels.

The protein consists of 81 amino acids, includes the PKA-binding amphipathic helix and N-terminal myristoyl and palmitoyl lipid anchors anchoring the PKA-AKAP complex on the plasma membrane. Interaction between AKAP18 and the Ca²⁺ channel takes place via the C-terminal domain of the alpha₁-subunits of the channel, involving a leucine zipper-like motif. In this way, the PKA is positioned near an important phosphorylation site in the alpha₁-subunit, thereby allowing specific phosphorylation. Phosphorylation increases the open probability of the channel.

Further studies have shown that several splicing variants emerge from the AKAP18 gene, for which reason the above-described AKAP18 protein has also been designated AKAP18alpha (or AKAP15). Other splicing variants are AKAP18beta, AKAPgamma and AKAPdelta.

AKAP18beta consists of 104 amino acids and includes the same membranous binding domain as the alpha variant. An additional domain comprising 23 amino acids directs the protein to the apical plasma membrane in polarized epithelial cells.

The function of AKAP18beta is unclear up to now. In contrast to most AKAP proteins, AKAP18gamma consisting of 326 amino acids is also localized in soluble cell fractions. In mouse oocytes, it anchors PKA-RI subunits in the nucleus, suggesting involvement in the regulation of transcription. AKAP18delta is a protein comprising 353 amino acids, the RII-binding domain of which is virtually identical to that of the alpha-gamma variants. It is largely homologous to AKAP18gamma. AKAP18delta has been discovered during a search for AKAP proteins involved in the translocation of the aquaporin-2 (AQP2) water channel from intracellular vesicles into the apical plasma membrane of renal collecting duct cells (see below). It has been demonstrated that AKAP18delta is localized on AQP2-containing vesicles.

In addition, its distribution strongly resembles that of AQP2, and, following suitable stimulation of the renal cells, it is translocated to the plasma membrane together with AQP2, so that involvement in AQP2 translocation is presumed.

AQP2 translocation is induced by the antidiuretic hormone arginine-vasopressin (AVP or ADH). Formation of the hormone and secretion thereof into the bloodstream are controlled by osmoreceptors in the hypothalamus, which constantly monitor the osmolarity of the blood. If the osmolarity increases, e.g. as a result of reduced fluid intake of the organism, AVP will be synthesized and secreted into the bloodstream.

The starting signal for AQP2 redistribution is provided by binding of AVP to the vasopressin receptor (V2R) on the basolateral membrane side of principal cells of the renal collecting duct. The V2 receptor, which is a GPCR, actuates the above-described signal cascade so that formation of cAMP is increased.

As a result of the conformational change triggered by cAMP binding, the PKA anchored on the AQP2-containing secretory vesicles via AKAP18delta releases its catalytic subunits which phosphorylate the AQP2 water channels situated nearby. Such phosphorylation provides for the transport of the vesicles to and fusion with the apical plasma membrane.

In this way, the water permeability of the membrane is increased, and increasing amounts of water can be reabsorbed from primary urine situated on the other side of the apical membrane in the collecting duct, thus counteracting the initial stimulus of the signal chain.

In addition to AKAP18delta, other proteins are associated with the AQP2 vesicles, but—in contrast to AKAP18delta—do not reach the plasma membrane, possibly implying that the PKA anchored to the vesicle membranes via AKAP18delta is required not only for AQP2 phosphorylation, but also e.g. for phosphorylations regulating the transport process to the plasma membrane. A function of this type has also been demonstrated for the L-type Ca²⁺ channel-associated AKAP79 (see above).

As set forth above, it is possible to inhibit binding of PKA to AKAP proteins by means of synthetic peptides mimicking the amphipathic helix of the PKA-binding domain. Although the above-mentioned inhibitory peptides play an important role in elucidating the physiological significance of PKA compartmentation by AKAP proteins, they have disadvantages. Being peptides, they are not particularly stable and are costly in synthesis, for example. Since AKAP proteins provide for specificity in many cellular processes and are expressed in a tissue-specific fashion, they represent an attractive target for new pharmaceutical agents. However, the AKAP proteins must be validated as target before developing new active substances. As a result, the interaction between AKAP proteins and PKA must be eliminated in animal models and subsequently in a human model as well. However, peptides cannot be administered to test animals, because peptides are degraded in the gastrointestinal tract. Other important models for the investigation of AKAP functions are cell culture models. However, in order to ensure membrane permeability, peptides must be acylated for use in cell cultures.

As a result of lacking steric hindrance, or owing to more properly interacting (hydrophobic) amino acid side chains, prior art peptides dock more tightly to the hydrophobic pockets of regulatory PKA subunits than complete AKAP proteins. In this way, the physiological effect of eliminating the PKA compartmentation by AKAP proteins can be tested in experiments.

The anchor inhibitor peptide first introduced and most frequently used to date is the Ht31 peptide which consists of 22 to 24 amino acids and has been derived from the PKA-binding domain of the Ht31 AKAP protein, also known as AKAPLbc.

Using methods of bioinformatics and peptide array screenings wherein, starting with a consensus binding site for RII subunits calculated from various AKAP proteins, amino acids are systematically substituted and peptides for binding experiments coupled to cellulose membranes, it was possible to generate a peptide, AKAP_{IS} (in silico), having a stronger inhibiting effect. It is a 17mer capable of undergoing additional hydrophobic interactions with the RII subunit as a result of modified positions of two amino acid side chains, and it can form an additional salt bridge stabilizing its conformation.

Starting from the RII binding domain of AKAP18delta included among the high-affinity AKAP proteins, it was possible to generate inhibitory peptides having even higher affinity. Among other things, they have the advantage of allowing lower dosing, rendering non-specific effects of the peptide more unlikely.

Here, all amino acids of a 25mer containing the AKAP18delta-RII binding domain were replaced one by one with all sorts of other biogenic amino acids. Starting from peptides with higher affinity, further amino acid substitutions based on a structural model of peptide binding in the hydrophobic pocket were carried out. However, further affinity enhancement was not possible. The resulting peptides were tested for their specificity and biological function as global inhibitors of AKAP-RII binding.

Surprisingly, it was found that the non-peptidic molecules in accordance with Table A can be employed as inhibitors or decouplers of PKA and AKAP or as substances blocking PKA anchoring. The non-peptidic inhibitors also represent lead structures for new active substances. On the one hand, such substances represent a new tool for blocking PKA anchoring in in vitro experiments and, on the other hand, represent the basis of a new class of pharmaceutical agents which, in contrast to traditional pharmaceutical agents, affect protein-protein interactions rather than enzyme or receptor activity.

All these molecules have in common that they are non-peptidic blockers or decouplers of PKA-AKAP interaction, which have not been disclosed in the prior art as yet. All these substances can also be used to block PKA anchoring. The compounds claimed herein allow specific inhibition of an AKAP-RII complex. Surprisingly, the chemical and physical properties of the substances according to the invention enable direct use in cell cultures, animal models, as well as in the field of primates and humans, thereby allowing in vivo investigations on the function of AKAP proteins for the first time. To date, in vivo investigations on the function of AKAP proteins have not been described in the prior art. This in vivo suitability is a common feature of all compounds according to the invention.

Not all of the inventive compounds exhibit a new structural element, but this does not imply lacking unity of the teaching according to the present application. The alternative chemical compounds claimed herein have a common property or effect.

In another aspect of the invention, preferred compounds claimed herein surprisingly exhibit a variety of common physicochemical properties which make the compounds highly useful as pharmaceutical agents because they comply with Lipinski's Rule (the so-called Rule of Five) to a very large extent. The above-mentioned common physicochemical or structural features of the components according to the invention relate to their molecular weight which is in the range of from 150 to 600 g/mol, preferably from 190 to 300 g/mol, a partition coefficient of log P≦10, preferably ≦8, more preferably ≧1 to ≦5, with a maximum of 10 hydrogen bridge donors and a maximum of 10 hydrogen bridge acceptors, a solubility value of log Sw of from −400 to 0, and a BrotN value of from 0 to 7, the AKAP18delta-RII interaction preferably being inhibited by at least 40%. The sum of structural common features results in a functional relationship of inhibiting the effect of PKA and AKAP and blocking PKA anchoring. The common physicochemical features therefore do not represent an arbitrary sum of features, but—so to speak—the common fingerprint of the claimed compounds, which advantageously enables and characterizes good in vivo suitability of the compounds. In a particularly preferred fashion the molecules of the invention bind to regulatory subunits of PKA, particularly to RIalpha or RIIalpha and RIIalpha or RIIbeta, respectively. The molecules of the invention allow modification, inhibition or decoupling of AKAP, preferably AKAP18, more preferably AKAP18delta, and PKA in dependence on the species being used. Using simple routine tests, a person skilled in the art can easily provide recognition molecules directed to the molecules according to the invention. For example, these can be antibodies, chelators, complexing agents, or other structures well-known in the prior art. Such recognition molecules are easy to generate, provided their target is known, i.e., the decouplers in the present case. For example, the decouplers can be administered in organisms together with an adjuvant, thereby forming antibodies which can be collected according to well-known methods. Using these recognition molecules, or using the molecules of the invention, organisms wherein the AKAP-PKA interaction is modified in a tissue- and/or cell-specific fashion can be provided by contacting the organisms with the inventive molecules or with the recognition molecules.

Preferred decouplers have a log P value of <9, 8, 7, 6, 5, preferably 4, more preferably 3, and especially preferably <2, and 10, 9, 8, 7, 6, preferably 5, especially preferably 4, 3 and/or 2 hydrogen bridge donors and/or acceptors at maximum and/or a BrotN value especially of 1, 2, 3, 4, 5, or 6 and/or a log Sw value of 350, 300, 250, 200, 150, 100 or 50, and a molecular weight of from 150 to 550, from 150 to 500, from 150 to 450, from 150 to 400 or from 150 to 350.

The decouplers preferably have 6 hydrogen bridge donors at maximum and/or 4 hydrogen bridge acceptors at maximum and/or a partition coefficient log P of ≧1 to ≦5.

In a particularly preferred embodiment of the invention the inventive decouplers inhibit the interaction of AKAP and PKA subunits by at least 80%. Adequate methods by means of which the inhibition of interaction can be determined are well-known to those skilled in the art. For example, there are numerous disclosures describing how to accomplish inhibition of AKAP-PKA binding with the aid of the Ht31 peptide. In the meaning of the invention, however, inhibition implies any form of modification of the interaction between AKAP and PKA compared to a non-influenced interaction between the two molecules. Although inhibition of binding between AKAP and PKA is preferred, increasing the interaction of AKAP and PKA may also be desired according to the invention.

Particularly preferred decouplers are those represented in Table A. The decouplers therein have physicochemical properties resulting not only in inhibition of the interaction of AKAP and PKA, but also in very good absorption thereof in a target organism, thus allowing treatment of diseases induced by an imbalance or disorder of the cAMP-dependent signal transduction. A person skilled in the art will be familiar with such diseases from the present state of the art and will therefore understand which kinds of diseases are covered and concerned. Well-known diseases are, for example, insipid diabetes, pancreatic diabetes, obesity, edema, chronic obstructive pulmonary diseases, AIDS, schizophrenia, hepatic cirrhosis, heart failure, coronary heart diseases, hypertonia and/or asthma. However, the diseases caused by disorders in the cAMP-dependent signal transduction are not limited thereto. Further diseases as mentioned below are also included among diseases highly susceptible to treatment with the agents according to the invention.

Other preferred molecules according to the invention are disclosed in Table B. These preferred compounds have physicochemical properties in common which enable the use of said compounds, optionally together with a pharmaceutically acceptable carrier, in surgical and/or therapeutic treatment of a human or animal body and in diagnostic methods carried out on a human or animal body. Advantageously, the compounds have substance properties complying with the Rule of Five by Lipinski et al. (Lipinski et al., Adv. Drug Deliv. Rev. 46, 3-26, 2001).

Another preferred embodiment of the invention relates to decouplers selected from Table C. The preferred compounds therein have such good membrane permeability that they can easily be used in vivo, i.e. especially as pharmaceutical agents, to block PKA anchoring or modulate, particularly inhibit, the AKAP-PKA interaction.

Advantageous molecules in accordance with the invention are disclosed in Tables A, B and/or C. Owing to their physicochemical properties, such as low molecular weight, advantageous partition coefficient, and solubility and BrotN values, and to the fact that no more than 10H bridge acceptors and, in particular, essentially no more than 7, preferably 6, more preferably 5H bridge donors are included, these agents are highly useful in prophylaxis, therapy, follow-up and/or diagnosis in in vivo systems, such as target organisms, preferably in humans or in test animals, such as primates, rats or mice. Other preferred decouplers are disclosed in Table D.

In another preferred embodiment of the invention the inventive decouplers have the general formula I wherein mesomeric interconversion may take place (R² and R³ are regarded as interchangeable)

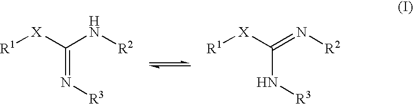

wherein X is a non-hydrogen atom, preferably a sulfur atom, R¹ is an alkyl or aryl residue, preferably a 1-naphthylmethyl residue, R² and R³ are hydrogen atoms or alkyl or aryl residues, and R² and R³ are preferably two hydrogen atoms, two methyl residues, one benzyl residue and one methyl residue or one benzyl residue and one tert-butyl residue, and in a particularly preferred fashion, R² and R³ are a 2-thiazolidinyl residue and a methyl or tert-butyl residue, as well as a 1-naphthyl residue and an isopropyl, cyclohexyl, benzyl or methyl residue. Accordingly, the invention also relates to compounds in accordance with general formula I for use as drugs or pharmaceutical active substances. In another preferred embodiment the invention also relates to the use of the compounds in accordance with general formula I for the treatment of diseases as disclosed in the present application.

In another preferred embodiment of the invention, the inventive decouplers have the general formula II wherein R¹ to R³ have the meaning as above:

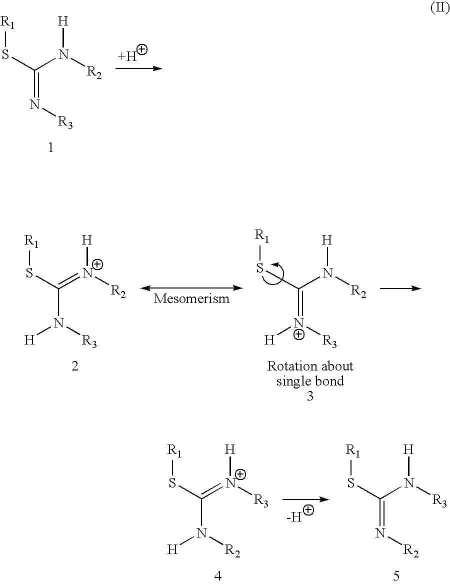

The ratio 1/5 depends on the basicity of the nitrogen adjacent to R³. Advantageously, the position of the R² and R¹ residues is interchangeable from structure 2 on. Protonated compounds, e.g. a hydrochloride, are particularly preferred for use as decouplers of the AKAP-PKA interaction. In the event of compounds having multiple basic centers, e.g. compound JG5 (see Table), the dihydrochloride is particularly preferred. Preferred is the lead structure 990 (see Table).

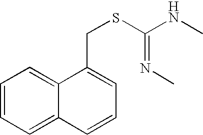

The IC values decrease with increasing size of the aliphatic residues, and—as is the case with JG31 (see Table)—the anthracene exhibits additional effects. This implies higher electron shift towards the amidine and higher basicity. The electron density on the amidine is important in particular uses, so that the residues R¹ and R² are electron-donating moieties (see compounds 6 and 7; ortho- or para-methoxyphenyl, ortho- or para-diaminophenyl).

Particular degradation products of the decouplers according to the invention may also be advantageous. Under certain conditions, thiocarbamidines (such as No. 990) are unstable towards nucleophiles such as amines, forming thiols and guanidines which can also be used for the treatment of diseases, for example. In a preferred embodiment, the invention therefore relates to the cyclic imidazoles (6) and the dihydroimidazoles (7) for use in medicine, especially as decouplers of AKAP and PKA, and as tools in basic research in vivo and in vitro. The residues R¹ and R² are preferably aryl residues or cycloaliphatic residues (SM61, SM63 and SM65). Also preferred is the 6-membered ring on the amidine (compound 8 is based on SM71).

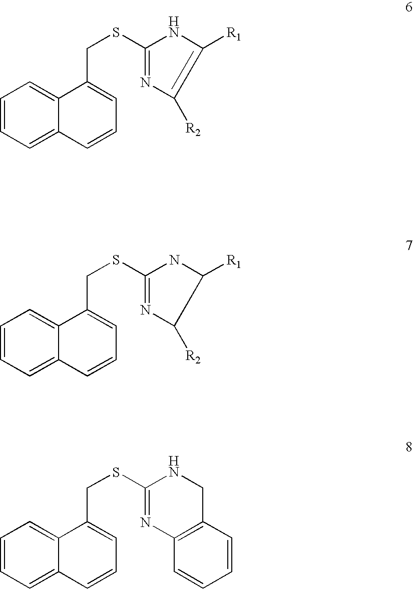

It is also advantageous when the amidine is separated from the sulfur and attached in the 2-position of the naphthalene (compound 9). In an advantageous embodiment, the distance N—S remains virtually the same. R¹ preferably contains S—R, so that the electron density on the amidine is comparable to that of the active compounds. R is preferably an alkyl residue. Accordingly, the isomer 10 is also preferred.

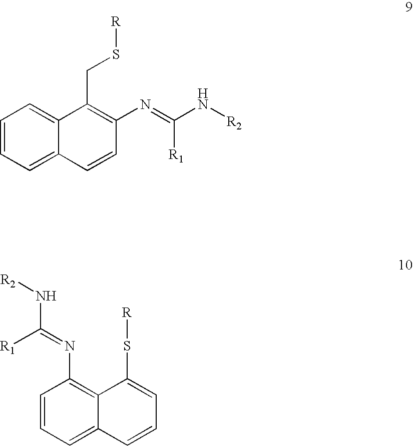

Therefore, decouplers in accordance with general formulas 11 and 12 are preferred.

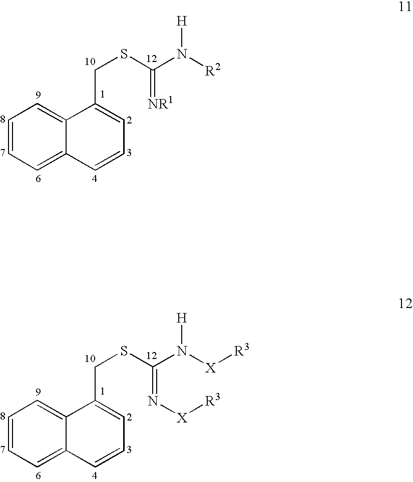

Claimed herein are the compounds per se, as well as their HCl salts. In preferred embodiments, R¹ and R² independently can be
acyclic aliphatic, with a chain length of from C₁ to C₆;
cycloaliphatic, with a ring size of from C₃ to C₉, with one or more O- or N-type heteroatoms independently of each other being included;
aromatic, as a monocyclic, heteroaryl and mono- to trisubstituted monocyclic residue.

The electron-donating substituents (e.g. OMe or NMe₂) were alluded to above. The use of heteroaromatics is particularly advantageous with regard to bioavailability. It is also advantageous to use structure 12 with X as acyclic aliphatic type linker group with a chain length of from C₁ to C₆ and R³ with the same groups as for R¹ and R² formulated independently of each other.

As for the chemistry of the above-mentioned compounds, it should be noted that the sulfur and the adjacent position 10 can be sensitive to oxidation in some embodiments of the invention. In those cases where only sulfur is oxidized, it is advantageous when the active form does not have the backbone of the lead structure (see above), but is either the sulfoxide 2 or the sulfone 3. Moreover, the position 10 may be acidic after oxidation of the sulfur and, following deprotonation, may undergo e.g. a Claisen aldol addition. This latter reaction is exemplified using an acetic acid building block as compound 16 (see below). If R² is a hydrogen atom, intramolecular ring closure also takes place.

Advantageously, the above-mentioned two positions (sulfur and No. 10) are highly reactive.

For structure 11 to be effective, it is preferred not to modify position 10. As set forth above, the latter can be acidic and—as explained below—can be sensitive to oxidation. Advantageously with no significant change in bulkiness, this position can be provided with fluorine atoms preventing diversification under physiological conditions and advantageously retaining the activity. Structure 16, in particular, is thus protected from premature loss.

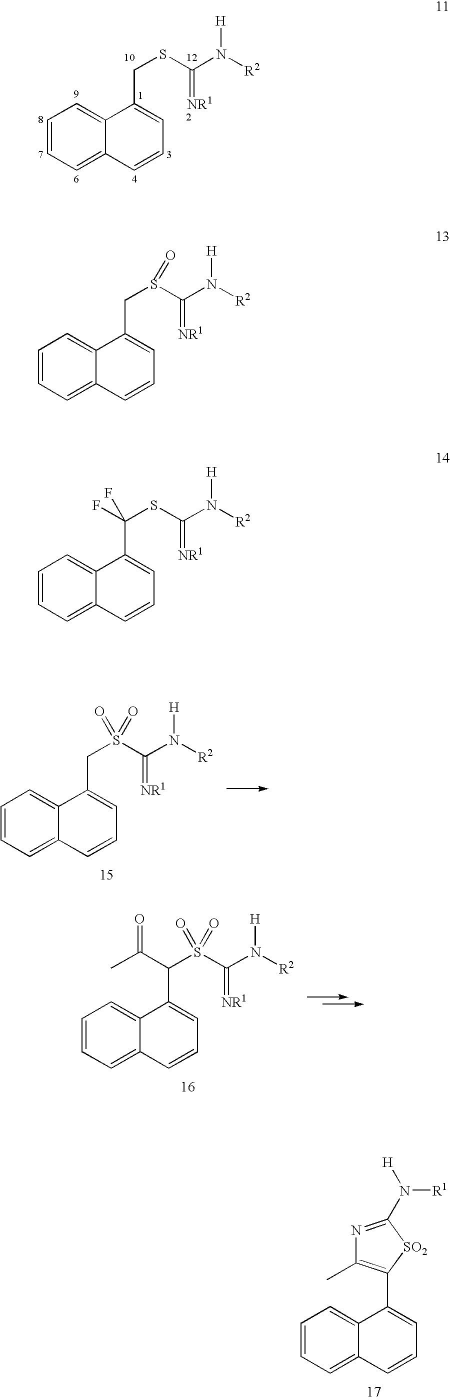

As a preferred embodiment of resultant products from the oxidation of position 10 (oxidation yields compound 18), the product of addition of an acetyl moiety as structure 8-following elimination of water—is exemplified in 20 (see below). Advantageously, position 10 can also be substituted. In particular, this involves the residues R³ and R⁴ as illustrated in compound 21, which, as an extension of R¹ and R², can also be F. The above explanations relating to the spacers between N and R¹/R² and oxidation on the sulfur also apply to the compounds listed below.

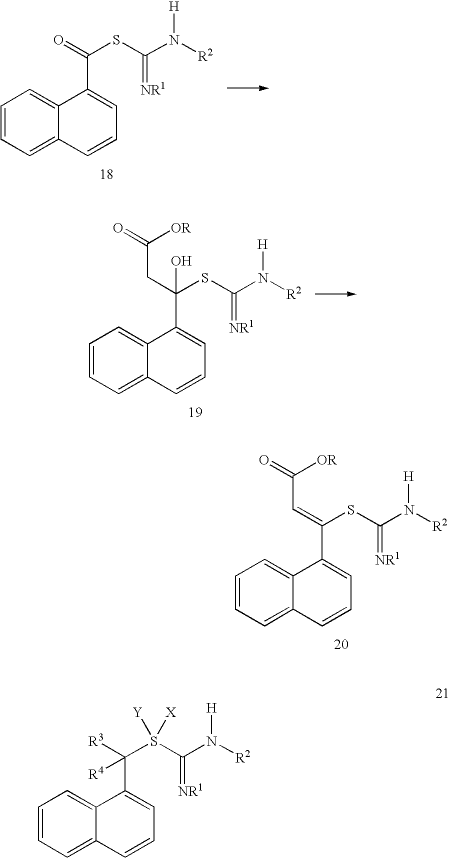

For example, R¹, R², R³ and R⁴ can independently represent alkyl and/or aryl residues.

Accordingly, the use of 1-substituted naphthalenes is preferred. In the lipophilic pockets of a target molecule, the affinity thereof to a lipophilic side group, such as an aromatic bicyclic unit, may afford a significant contribution to the acceptance of the substrate. Accordingly, compounds having the general formula in accordance with the compounds 22, 23 and 24 are also preferred and advantageously exhibit equivalent affinity.

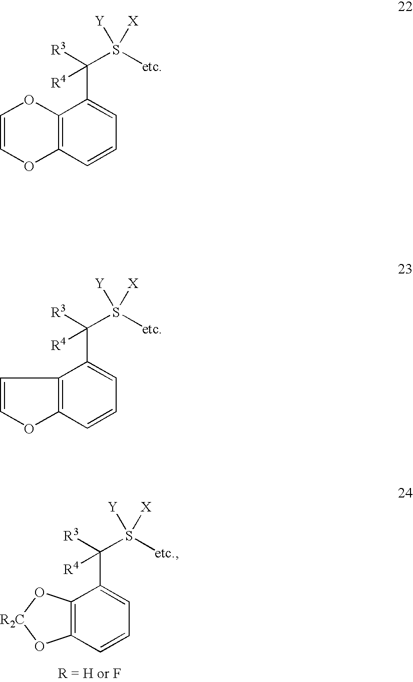

In advantageous embodiments, such bicyclic units in accordance with compounds 22 to 24 have X=oxygen, but also N, NH or S. Triannelated ring systems can also be preferred (see also compounds SM39 and SM44 in the Table).

In another preferred embodiment, guanidines are preferred, if R¹ or R² is cyano (for example, compound 25).

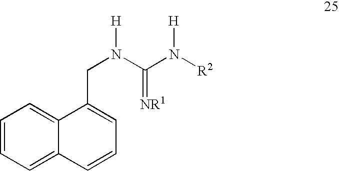

Also preferred is a sulfur amidine which may have different stability in vivo and in vitro (lead structure in accordance with compound II). In oral application, the compounds according to the invention have a water solubility of 1 mg/ml, preferably at a pH value of 2 to 7. When injecting a daily dose of 200 to 400 mg, the water solubility is even higher so as to avoid injection of 400 ml of solution with one syringe.

Therefore, a compound is also preferred wherein naphthalene is replaced by quinoline or isoquinoline, which can be administered as HCl salt, for example, particularly to increase the water solubility.

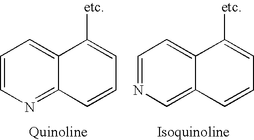

Accordingly, a decoupler of general formula III is preferred, i.e. in accordance with structure 1′ and formula 2′ which is in equilibrium with the former.

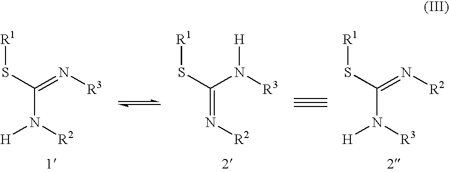

When 2′, 2″ are rotated about the S—C single bond and compared with 1′, the double bond and the hydrogen will coincide, as is the case with R² and R³ (and vice versa).

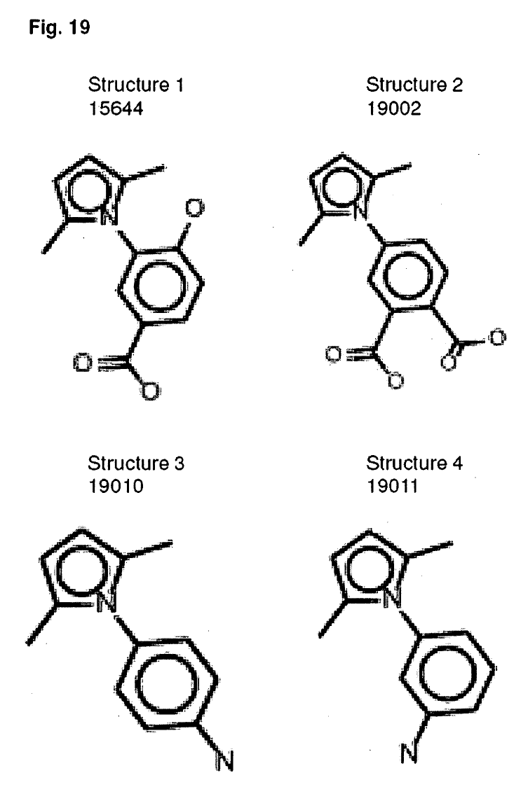

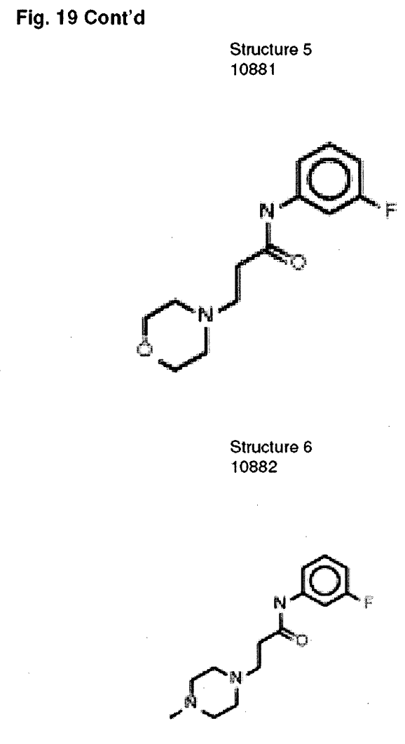

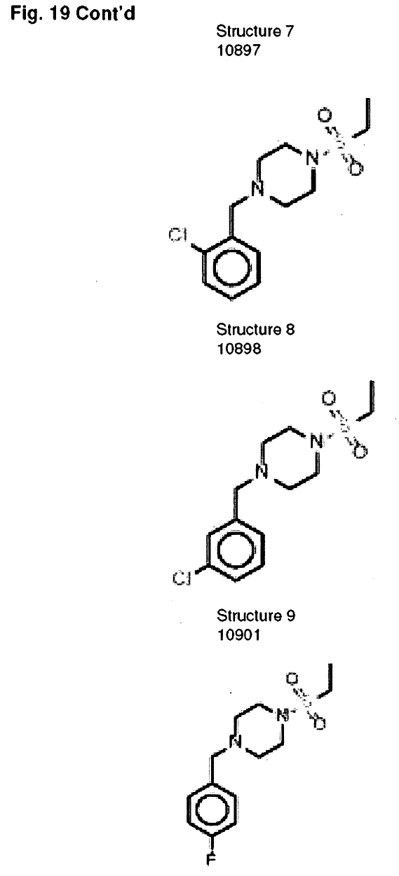

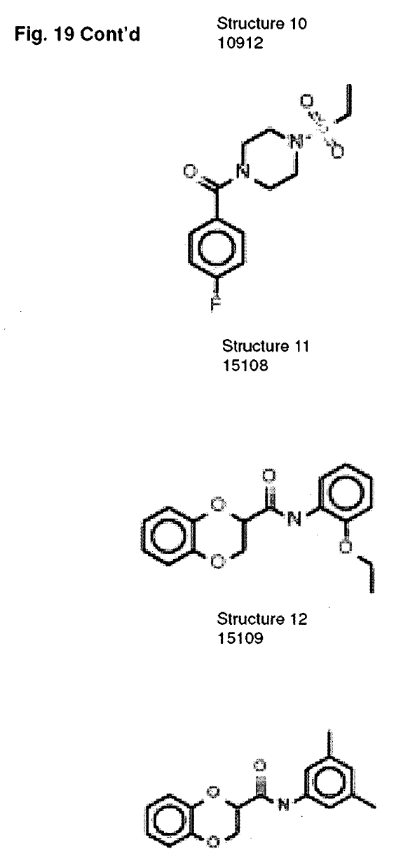

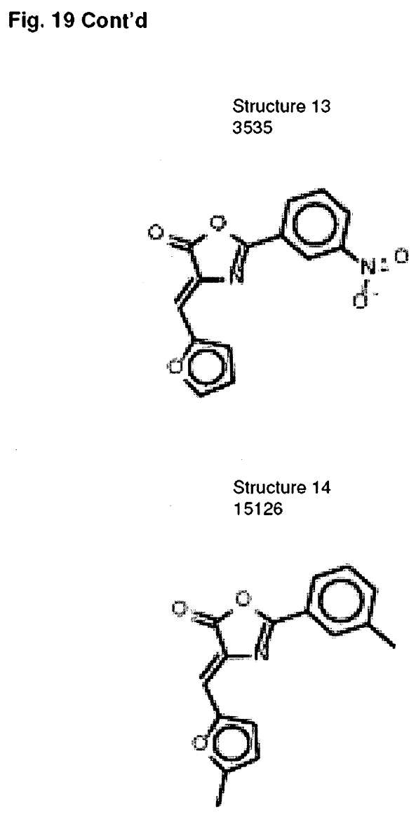

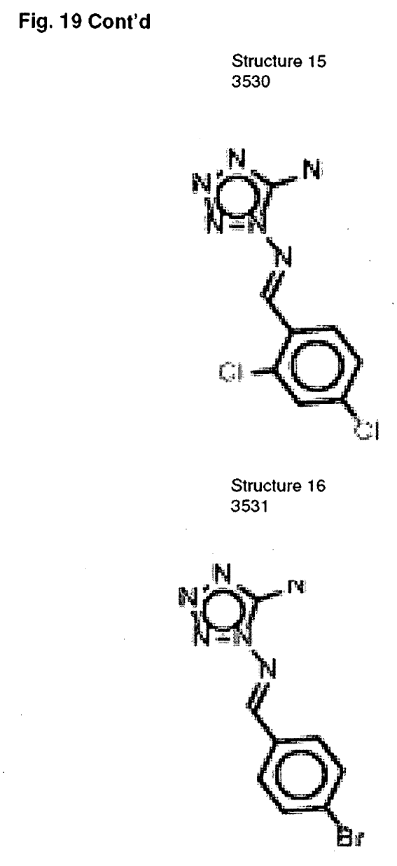

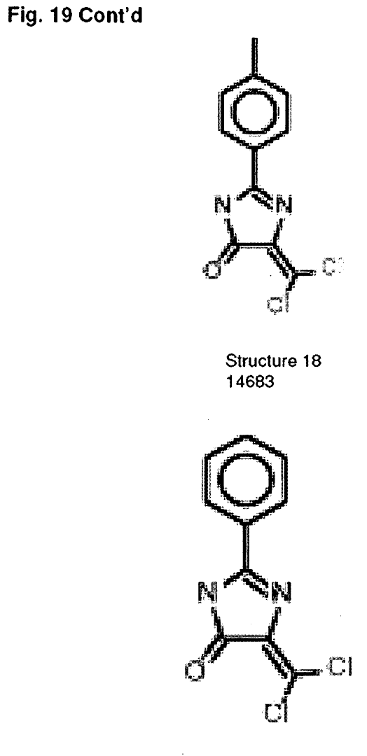

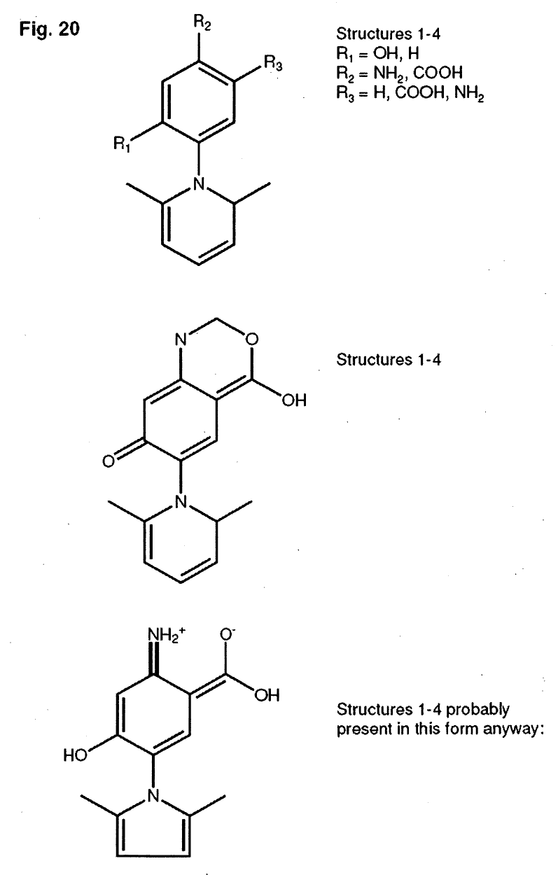

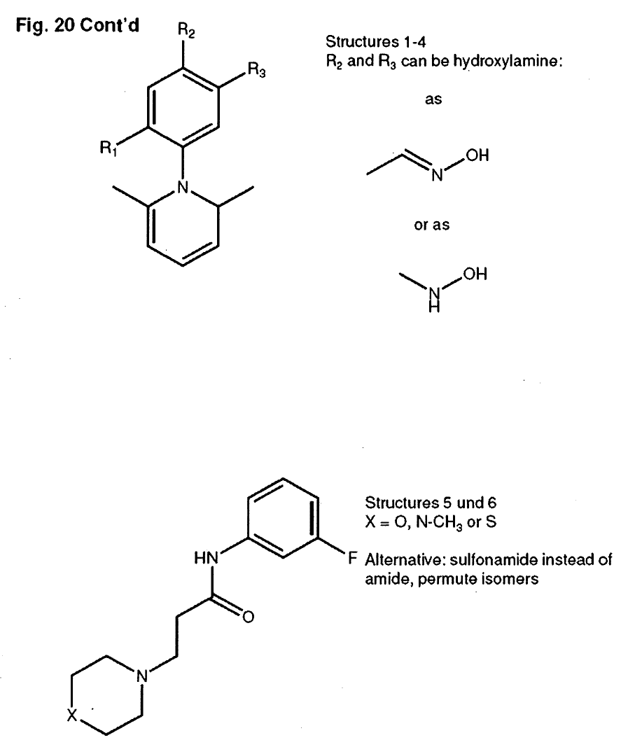

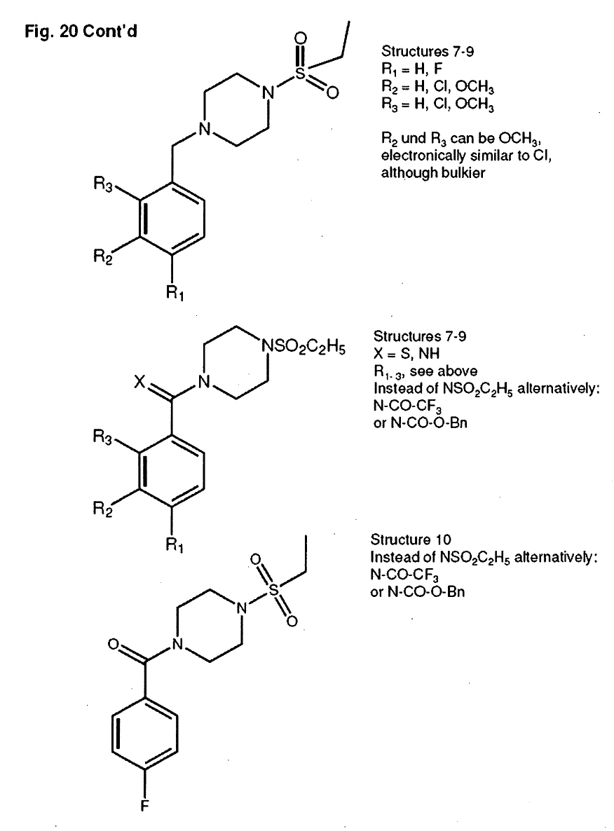

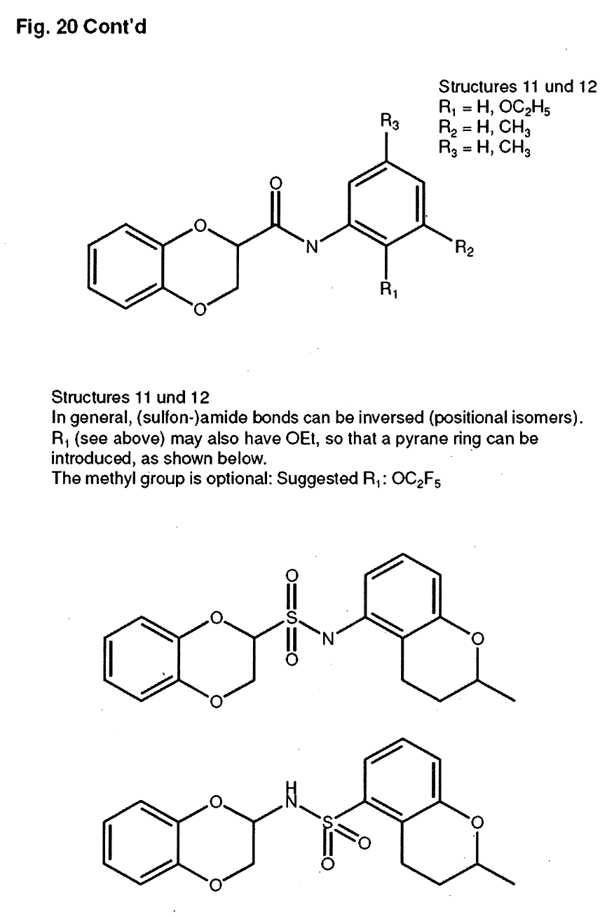

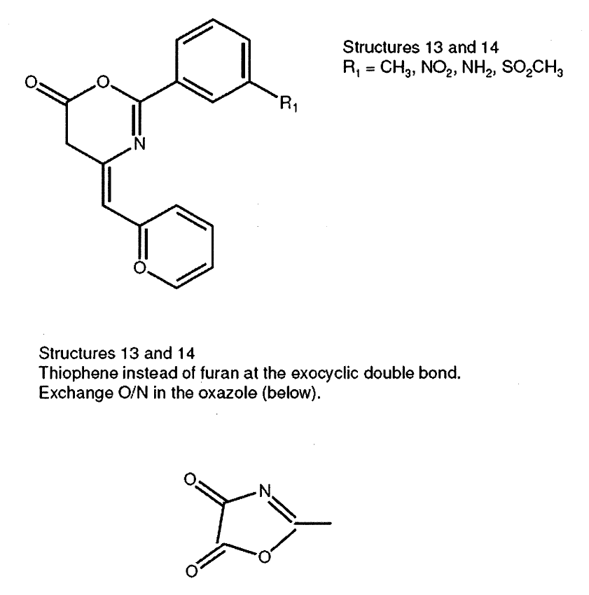

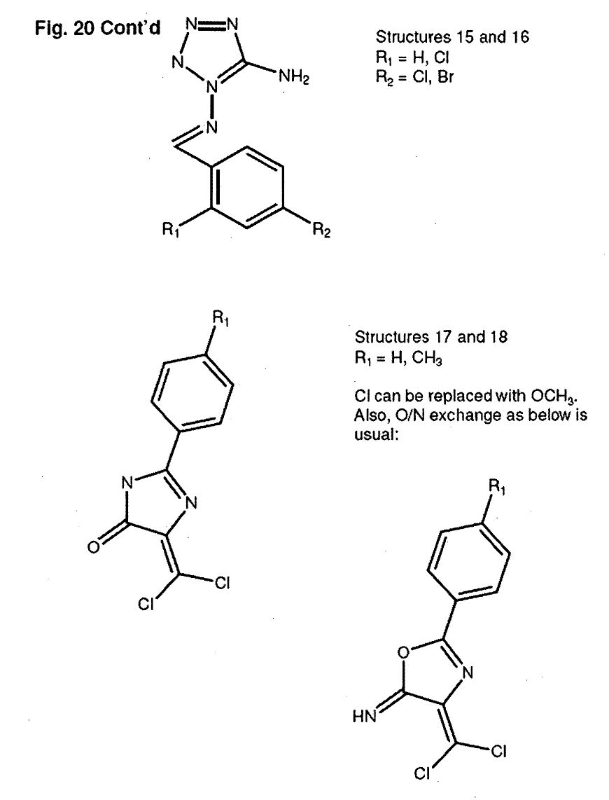

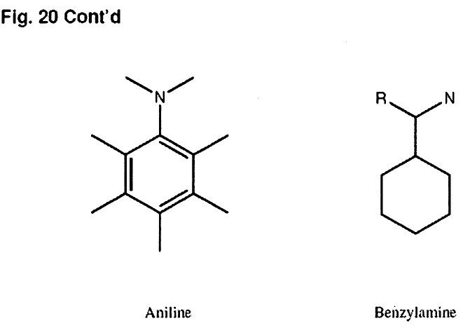

Other preferred decouplers have a general structure in accordance with FIG. 19 and/or FIG. 20. Preferred decouplers have many surprising advantages over the peptidic inhibitors or decouplers known from the prior art. First of all, the non-peptidic decouplers according to the invention represent a departure from conventional technologies, corresponding to a new field of problems. The structures according to the invention satisfy a long-unresolved, urgent need on which hitherto vain efforts have been made in the art. In particular, the simplicity of the solution indicates inventive activity, as it replaces more complicated teachings of the prior art. The development in scientific technology relating to the treatment of the diseases mentioned above and below has proceeded in a different direction, so that the teaching of the invention represents an achievement that rationalizes development and eliminates erroneous ideas in the art on the solution of the problem at issue. More specifically, the technical progress achieved by the teaching of the invention can be seen in improvement, performance enhancement, lower expense, savings of time, materials, work steps, cost or raw materials difficult to obtain, enhanced reliability, elimination of flaws, superior quality, maintenance freedom, greater efficiency, higher yield, expansion of the technical scope, provision of a further means, creation of another approach in the treatment of diseases, creation of a new field, first-time solution of a problem, provision of reserve means, alternatives, scope for rationalization, automation and miniaturization, and enrichment of the range of available drugs. Accordingly, the teaching of the invention represents a fortunate choice where one has been selected out of a variety of possibilities, the result of which has not been predictable. The teaching of the invention is a young field of technology which is praised in the art and leads to economic success and issue of licenses. In particular, the inventive molecules can be used in the treatment of diseases caused by a disorder or defect of the cAMP-dependent signal transduction. The above characterization of the representation of diseases is not intended to provide a functional definition of the diseases to be put to therapy; rather, the representation as diseases associated with a modification or a defect of the compartmentalized cAMP-dependent signal transduction serves as a generic term for a clearly defined group of diseases in the meaning of the invention. That is to say, a person skilled in the art can estimate which types of diseases are covered by the definition in accordance with the generic term and thus fall within the claims of the teaching according to the invention. Accordingly, citing the individual specific diseases falling under the generic term is not meant to claim a barely manageable number of possible therapies, with no tests being presented, but merely serves to clarify and specifically define the claimed diseases associated with a disorder of the above-mentioned signal transduction. Using the molecules according to the invention, it is possible to influence the above-mentioned AKAP proteins in their interaction with PKA molecules. More specifically, the above-mentioned AKAP18 proteins and their interaction with PKA molecules can be modified, especially AKAP18delta proteins and their interaction with PKA molecules, especially with subunits and especially preferably with RIIalpha and/or RIIbeta molecules. That is, for example, the interaction of AKAP79, gravin, AKAP82 or other AKAPs mentioned above, especially of AKAP18 such as AKAP18alpha, -beta, -gamma, -delta, especially preferably of AKAP18delta, can be modulated, especially inhibited, in their interaction with PKA. In a preferred fashion the decouplers essentially inhibit 100%, with inhibition of 90%, 80%, 70%, 60%, 50%, 40% also being preferred, more preferably of 30%, especially preferably 20%, and particularly 10%. Each of the above percentages of inhibition can be preferred.

On the one hand, the decouplers of the invention are claimed as new molecules; on the other hand, preferred molecules are claimed as compounds disclosed as finding use in medicine for the first time.

In another preferred embodiment of the invention, the new molecules, especially the new molecules in the field of medicine, are claimed for the treatment of diseases which, in accordance with the definition of the invention, fall under the term of diseases associated with compartmentalized cAMP-dependent signal transduction.

In a particularly preferred embodiment, this concerns diseases selected from insipid diabetes, pancreatic diabetes, obesity, edema, chronic obstructive pulmonary diseases, AIDS, schizophrenia, hepatic cirrhosis, heart failure, coronary heart diseases, hypertonia, duodenal ulcer and/or asthma.

The compounds according to Table B, preferably Table C or D, are new molecules which have not yet been described in the prior art. The other compounds (preferably according to Table A) disclosed have not been disclosed as being useful in the sector of therapeutic or diagnostic methods. However, these compounds indeed can be interpreted as completely new compounds even though their novelty is only given by the fact that they had been recorded in an archive or library, but were unrecognized by the general public due to lack of cataloguing (see especially Tables A and B).

The invention also relates to recognition molecules targeted to the non-peptidic molecules according to the invention. Owing to the disclosure of the non-peptidic molecules of the invention, a person of ordinary skill in the art can provide recognition molecules for the inventive non-peptidic molecules without unconscionable efforts, using routine tests, so that the recognition molecules are clearly and completely disclosed. According to the invention, the recognition molecules are antibodies, complexing agents or chelators or peptides interacting with the non-peptidic decouplers in such a way that the biological activity thereof, i.e. modulating the AKAP-PKA interaction, is not impaired. The recognition molecules also allow detection of the decouplers in an in vivo or in vitro system. To this effect, providing the recognition molecules with a detectable probe can be advantageous. Such probes are well-known to those skilled in the art.

The invention is also directed to cells, cell aggregates, tissue cultures or tissue patches, but also organisms, such as mice, rats, cattle, horses, donkeys, sheep, camels, goats, pigs, rabbits, guinea pigs, hamsters, cats, monkeys, dogs, or humans, comprising the decouplers of the invention and/or the recognition molecules of the invention. Using these cells, tissue cultures or organisms, it is possible to investigate various diseases, such investigations of diseases relating e.g. to the causes thereof or to possible methods of diagnosis and treatment. Preferred diseases are asthma, hypertonia, hypertrophy of the heart, coronary heart diseases, duodenal ulcer, heart failure, hepatic cirrhosis, schizophrenia, AIDS, pancreatic diabetes, insipid diabetes, obesity, chronic obstructive pulmonary diseases, learning disorders, edema (pathological water retention), infectious diseases and/or cancer. Of course, using an organism in a study of such diseases which does not have the recognition molecules of the invention, but has the decouplers of the invention or both structures at the same time, can also be preferred. Based on the disclosure of the teaching according to the invention, a person skilled in the art will know which type of decouplers or recognition molecules must be used at which concentration, because the latter immediately and unambiguously follows—by means of routine tests—from the disclosed technical teaching of the invention and from the prior art as explained e.g. in reference books. The organisms can be used in the development of pharmaceutical agents which modify, preferably decouple, the PKA-AKAP interaction. Obviously, the inventive decouplers themselves can be used as test molecules for pharmaceutical agents, but also as lead structures from which pharmaceutical agents are developed. The organisms also allow in vivo investigations of metabolic processes where PKA-AKAP interaction plays a role, or which processes require clarification as to whether AKAP-PKA interaction is involved in a particular incident, such as a particular pathogenic change, e.g. degeneration of cells. The inventive decouplers or recognition molecules can also be modified decouplers or recognition molecules obtained from the compounds according to the invention by means of combined methods. Essentially, the structures thus obtained, in which the molecules of the invention serve as lead structures, are functionally analogous to the decouplers and recognition molecules according to the invention. “Functionally analogous” means that the homologous structures obtained likewise allow conclusions as to the interaction of AKAP and PKA or the significance thereof to particular diseases. Accordingly, functionally analogous molecules in the meaning of the invention are molecules which can be identified by a person skilled in the art as having essentially the same effects. Accordingly, the invention is also directed to modified molecules having essentially the same function on essentially the same route, furnishing essentially the same result as the inventive decouplers or recognition molecules, but also to those equivalent compounds making it obvious to a person of ordinary skill in the art that they would achieve the same as the molecules disclosed in the claims. Accordingly, the above functionally analogous structures are obtained by using the decouplers or recognition molecules of the invention as lead structures. The functional analogs can be obtained using a structure-based, combined or other drug design. The term “drug design” is clearly defined to a person skilled in the art, relating e.g. to the reference “Wirkstoffdesign. Der Weg zum Arzneimittel.” or “Lehrbuch der klinischen Pharmazie” or other standard text books.

Accordingly, the invention also relates to a pharmaceutical agent comprising an inventive decoupler, or a recognition molecule targeted thereto, in the form of a chelator, complexing agent or antibody, optionally together with a pharmaceutically acceptable carrier and/or auxiliary agents. For example, the auxiliary agents can be adjuvants, vehicles or others. For example, the carriers can be fillers, diluents, binders, humectants, disintegrants, dissolution retarders, absorption enhancers, wetting agents, adsorbents and/or lubricants. In this event, i.e. if carriers, adjuvants and/or vehicles, such as liposomes, are present together with the inventive decouplers or recognition molecules thereof, they will be referred to as drug or pharmaceutical agent.

In another preferred embodiment of the invention the agent according to the invention is formulated as a gel, poudrage, powder, tablet, sustained-release tablet, premix, emulsion, brew-up formulation, drops, concentrate, granulate, syrup, pellet, bolus, capsule, aerosol, spray and/or inhalant and/or used in this form. The tablets, coated tablets, capsules, pills and granulates can be provided with conventional coatings and envelopes optionally including opacification agents, and can also be composed such that release of the active substance(s) takes place only or preferably in a particular area of the intestinal tract, optionally in a delayed fashion, to which end polymer substances and waxes can be used as embedding materials.

For example, the drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate can also be added. For oral administration in the form of capsules, diluents that can be used include lactose and dried corn starch. In oral administration of aqueous suspensions the active substance is combined with emulsifiers and suspending agents. Also, particular sweeteners and/or flavors and/or coloring agents can be added, if desired.

The active substance(s) can also be present in microencapsulated form, optionally with one or more of the above-specified carrier materials.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silica, talc and zinc oxide or mixtures of these substances.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substances, i.e., the compounds according to the invention, solutions and emulsions may include conventional carriers such as solvents, solubilizers and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substances, suspensions may include conventional carriers such as liquid diluents, e.g. water, ethyl alcohol, propylene glycol, suspending agents, e.g. ethoxylated isostearyl alcohols, polyoxyethylenesorbitol and sorbitan esters, microcrystalline cellulose, aluminum metahydroxide, bentonite, agar and tragacanth or mixtures of these substances.

The drugs can be present in the form of a lyophilized sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their polyoxyethylated forms. Such oil solutions or suspensions may also include a long-chain alcohol or similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the compounds according to the invention should be present in the above-mentioned pharmaceutical preparations at a concentration of about 0.01 to 99.9 wt.-%, more preferably about 0.05 to 99 wt.-% of the overall mixture.

In addition to said compounds, the above-mentioned pharmaceutical preparations may include further pharmaceutical active substances, but also, in addition to said further pharmaceutical active substances, salts, buffers, vitamins, sugar derivatives, especially saccharides, enzymes, vegetable extracts and others. Buffers and sugar derivatives advantageously reduce the pain during subcutaneous application, and enzymes such as hyaluronidase increase the effectiveness. The production of the pharmaceutical formulations specified above proceeds in a usual manner according to well-known methods, e.g. by mixing the active substance(s) with the carrier(s).

The above-mentioned preparations can be applied in humans and animals in an oral, rectal, parenteral (intravenous, intramuscular, subcutaneous), intracisternal, intravaginal, intraperitoneal, local manner (powders, ointment, drops) and used in a therapy of the diseases specified below. Injection solutions, solutions and suspensions for oral therapy, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Moreover, the drugs can be incorporated in other carrier materials such as plastics (plastic chains for local therapy), collagen or bone cement.

In another preferred embodiment of the invention, the compounds are incorporated in a pharmaceutical preparation at a concentration of 0.1 to 99.5, preferably 0.5 to 95, and more preferably 20 to 80 wt.-%. That is, the compounds are pre-sent in the above-specified pharmaceutical preparations, e.g. tablets, pills, granulates and others, at a concentration of preferably 0.1 to 99.5 wt.-% of the overall mixture. Those skilled in the art will be aware of the fact that the amount of active substance, i.e., the amount of an inventive compound combined with the carrier materials to produce a single dosage form, will vary depending on the patient to be treated and on the particular type of administration. Once the condition of a patient has improved, the proportion of active compound in the preparation can be modified so as to obtain a maintenance dose that will bring the disease to a halt. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that a disease is combated as effectively as possible or the onset of such a disease is prevented by a prophylactic administration. Concentration and type of application can be determined by a person skilled in the art using routine tests. Preferred applications of the compounds of the invention are oral application in the form of powders, tablets, fluid mixture, drops, capsules or the like, rectal application in the form of suppositories, solutions and the like, parenteral application in the form of injections, infusions and solutions, and local application in the form of ointments, pads, dressings, douches and the like. Contacting with the compounds according to the invention is preferably effected in a prophylactic or therapeutic fashion.

For example, the suitability of the selected form of application, of the dose, application regimen, selection of adjuvant and the like can be determined by taking serum aliquots from the patient, i.e., human or animal, and testing for the presence of indicators of disease in the course of the treatment procedure. Alternatively or concomitantly, the condition of kidneys, liver, but also the amount of T cells or other cells of the immune system or of other markers characterizing or documenting the course of disease or convalescence can be determined in a conventional manner so as to obtain a general survey on the immunologic constitution of the patient and, in particular, the constitution of organs important to the metabolism. Additionally, the clinical condition of the patient can be observed for the desired effect. Where insufficient therapeutic effectiveness is achieved, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration.

In addition to oral ingestion, e.g. intramuscular or subcutaneous injections or injections into the blood vessels can be envisaged as other preferred routes of therapeutic administration of the compounds according to the invention. At the same time, supply via catheters or surgical tubes can be used, e.g. via catheters directly leading to particular organs such as kidneys, liver, spleen, intestine, lungs, etc.

In a preferred embodiment the compounds according to the invention can be employed in a total amount of preferably 0.05 to 500 mg/kg body weight per 24 hours, more preferably 5 to 100 mg/kg body weight. Advantageously, this is a therapeutic quantity which is used to prevent or improve the symptoms of a disorder or of a responsive, pathologic physiological condition.

Obviously, the dose will depend on the age, health and weight of the recipient, degree of the disease, type of required simultaneous treatment, frequency of the treatment and type of the desired effects and side-effects. The daily dose of 0.005 to 500 mg/kg preferably of 0.05 to 500 mg/kg body weight, can be applied as a single dose or multiple doses in order to furnish the desired results. In particular, pharmaceutical agents are typically used in about 1 to 10 administrations per day, or alternatively or additionally as a continuous infusion. Such administrations can be applied as a chronic or acute therapy. It will be appreciated that the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 10 times per day with the compounds of the invention or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can determine the optimum dosage required in each case and the type of application of the active substances.

In another particularly preferred embodiment of the invention the pharmaceutical agent is used in a single administration of from 1 to 100, especially from 2 to 50 mg/kg body weight. In the same way as the total amount per day (see above), the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in one application and which normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 2 tablets are administered in each oral application of the compounds of the invention. The tablets according to the invention can be provided with coatings and envelopes well-known to those skilled in the art or can be composed in a way so as to release the active substance(s) only in preferred, particular regions of the host.

It is preferred in another embodiment of the invention that the compounds according to the invention are optionally associated with each other or, coupled to a carrier, enclosed in liposomes, and, in the meaning of the invention, such enclosure in liposomes does not necessarily imply that the compounds of the invention are present inside the liposomes. Enclosure in the meaning of the invention may also imply that the compounds of the invention are associated with the membrane of the liposomes, e.g. in such a way that the compounds are anchored on the exterior membrane. Such a representation of the inventive compounds in or on liposomes is advantageous in those cases where a person skilled in the art selects the liposomes such that the latter have an immune-stimulating effect. Various ways of modifying the immune-stimulating effect of liposomes are known to those skilled in the art from DE 198 51 282. The lipids can be ordinary lipids, such as esters and amides, or complex lipids, e.g. glycolipids such as cerebrosides or gangliosides, sphingolipids or phospholipids.

Preferred diseases that can be treated with the agent according to the invention are selected from the group comprising AIDS, acne, albuminuria (proteinuria), alcohol withdrawal syndrome, allergies, alopecia (loss of hair), ALS (amyotrophic lateral sclerosis), Alzheimer's disease, retinal macula senile degeneration, anemia, anxiety syndrome, anthrax (milzbrand), aortic sclerosis, occlusive arterial disease, arteriosclerosis, arterial occlusion, arteriitis temporalis, arteriovenous fistula, arthritis, arthrosis, asthma, respiratory insufficiency, autoimmune disease, atrioventricular block, acidosis, prolapsed intervertebral disc, inflammation of the peritoneum, pancreatic cancer, Becker muscular dystrophy, benign prostate hyperplasia (BPH), bladder carcinoma, hemophilia, bronchial carcinoma, breast cancer, BSE, Budd-Chiari syndrome, bulimia nervosa, bursitis, Byler syndrome, bypass, chlamydia infection, chronic pain, cirrhosis, commotio cerebri (brain concussion), Creutzfeld-Jacob disease, intestinal carcinoma, intestinal tuberculosis, depression, diabetes insipidus, diabetes mellitus, diabetes mellitus juvenilis, diabetic retinopathy, Duchenne muscular dystrophia, duodenal carcinoma, dystrophia musculorum progressiva, dystrophia, ebola, eczema, erectile dysfunction, obesity, fibrosis, cervix cancer, uterine cancer, cerebral hemorrhage, encephalitis, loss of hair, hemiplegia, hemolytic anemia, hemophilia, pet allergy (animal hair allergy), skin cancer, herpes zoster, cardiac infarction, cardiac insufficiency, cardiovalvulitis, cerebral metastases, cerebral stroke, cerebral tumor, testicle cancer, ischemia, Kahler's disease (plasmocytoma), polio (poliomyelitis), rarefaction of bone, contact eczema, palsy, liver cirrhosis, leukemia, pulmonary fibrosis, lung cancer, pulmonary edema, lymph node cancer, (Morbus Hodgkin), lymphogranulomatosis, lymphoma, lyssa, gastric carcinoma, mammary carcinoma, meningitis, mucoviscidosis (cystic fibrosis), multiple sclerosis (MS), myocardial infarction, neurodermitis, neurofibromatosis, neuronal tumors, kidney cancer (kidney cell carcinoma), osteoporosis, pancreas carcinoma, pneumonia, polyneuropathies, potency disorders, progressive systemic sclerosis (PSS), prostate cancer, urticaria, paraplegic syndrome, traumatic, rectum carcinoma, pleurisy, craniocerebral trauma, vaginal carcinoma, sinusitis, esophagus cancer, tremor, tuberculosis, tumor pain, burns/scalds, intoxications, viral meningitis, menopause, soft-tissue sarcoma, soft-tissue tumor, cerebral blood circulation disorders and/or CNS tumors.

In another preferred embodiment the pharmaceutical agents of the invention can also be used in the treatment of cancerous diseases selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors may comprise the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchio-alveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarial carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In another preferred embodiment the cancerous disease or tumor being treated or prevented is selected from the group of tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated Hodgkin's disease and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prevented is selected from the group comprising mammary carcinomas, gastrointestinal tumors, including colon carcinomas, stomach carcinomas, pancreas carcinomas, colon cancer, small intestine cancer, ovarial carcinomas, cervical carcinomas, lung cancer, prostate cancer, kidney cell carcinomas and/or liver metastases.

In another preferred embodiment of the invention the disease is selected from the group comprising diseases referred to as infectious diseases in the meaning of the invention and associated with a modulation of the compartmentalized cAMP-dependent signal transduction, namely: monkey pox, AIDS, anthrax (Bacillus anthracis, milzbrand), avian influenza, borreliosis, Borrelia recurrentis, botulism (Clostridium botulinum), brucellosis, Campylobacter infections, chlamydial infections, cholera (Vibrio cholerae), Creutzfeldt-Jakob disease, Coxiella burnetii (Q fever), Cryptosporidium parvuum (cryptosporidiosis), dengue fever, diphtheria, ebola viral infections, echinococcosis (fox tapeworm, dog tapeworm), EHEC infections (STEC infections, VTEC infections), enterovirus, typhoid fever, (Rickettsia prowazeckii), Francisella tularensis (tularemia), spring-summer meningoencephalitis, yellow fever, giardiasis, gonorrhea, flu (influenza), Haemophilis influenzae, hantavirus, Helicobacter pylori, hepatitis C, hepatitis D, hepatitis E, herpes, HUS (hemolytic uremic syndrome), epidemic keratoconjunctivitis, pertussis, polio (poliomyelitis), infestation with head lice, infestation with itch-mites, Crimean-Congo fever, Lassa fever, food-related diseases, legionnaire's disease, leishmaniosis, lepra, leptospirosis, listeriosis, Lyme disease, Lymphogranuloma venereum, malaria (plasmodial infections), Marburg virus infections, measles, melioidosis, meningococcosis, MRSA (staphylococci), mumps, mycosis (fungus infections), new infectious diseases of increasing incidence, norovirus, ornithosis (parrot disease), papilloma ylrus, paratyphoid fever, plague (Yersinia pestis), pneumococcidal infections (Streptococcus pneumoniae), smallpox, travel-related infectious diseases, beef tapeworm infection in humans, rotavirus, German measles, RSV infections, salmonellosis, scarlet fever, severe acute respiratory syndrome (SARS), sexually communicable infections, shigellosis, syphilis, tetanus, rabies, toxoplasmosis, trichinosis, tuberculosis, typhoid fever, varicella (chickenpox), variant Creutzfeldt-Jakob disease, viral hemorrhagic fever, West-Nile fever, yersiniosis and/or diseases communicated by ticks. The agents according to the invention do not necessarily have to inhibit the enzymes of the above pathogens, particularly phosphatase. The agents may also have a membrane-destabilizing or other effect. In the meaning of the invention, reducing the pathogenicity of the pathogens is preferably essential.

In another preferred embodiment of the invention, the disease to be treated is essentially induced or co-induced by bacteria; said bacteria can be legionellas, streptococci, staphylococci, klebsiellas, Haemophilis influenzae, rickettsiae (typhoid fever), mycobacteria, mycoplasmas, ureaplasmas, neisseriae (meningitis, Waterhouse-Friedrichsen syndrome, gonorrhea), pseudomonads, bordetellas (pertussis), corynebacteria (diphtheria), chlamydiae, campylobacteria (diarrhea), Escherichia coli, proteus, salmonellas, shigellas, yersiniae, vibrions, enterococci, clostridiae, listeriae, borreliae, Treponema pallidum, brucellas, francisellas and/or Leptospira.

The invention also relates to the use of the decouplers for specific binding to AKAP, preferably AKAP18, more preferably AKAP18delta, and/or specific binding to PKA, preferably to subunits thereof, and more preferably to RII subunits.

The invention also relates to the inhibition of the interaction of RIalpha, RIIalpha, RIbeta and/or RIIbeta subunits of PKA with AKAP, with inhibition in the meaning of the invention being any type of modification.

In a particularly preferred embodiment of the invention, the decouplers can be used as aquaretic agent, contraceptive agent, anti-infective agent, anxiolytic agent and/or anti-tumor agent.

In another advantageous embodiment the diseases are selected from the group comprising any type of asthma, etiology or pathogenesis, or asthma from the group of atopic asthma, non-atopic asthma, allergic asthma, IgE-mediated atopic asthma, bronchial asthma, essential asthma, primary asthma, endogenous asthma caused by pathophysiologic disorders, exogenous asthma caused by environmental factors, essential asthma of unknown or unapparent origin, non-atopic asthma, bronchitic asthma, emphysematous asthma, stress-induced asthma, occupational asthma, infectious-allergic asthma caused by bacterial, fungous, protozoal or viral infection, non-allergic asthma, incipient asthma, “wheezy infant syndrome”;

chronic or acute bronchoconstriction, chronic bronchitis, obstruction of the small respiratory tract, and emphysema; any type of obstructive or inflammatory diseases of the respiratory tract, etiology or pathogenesis, or obstructive or inflammatory diseases of the respiratory tract from the group of asthma; pneumoconiosis, chronic eosinophilic pneumonia; chronic obstructive pulmonary disease (COPD); COPD including chronic bronchitis, pulmonary emphysema or associated dyspnoea, COPD characterized by irreversible, progressive obstruction of the respiratory tract, shock lung (adult respiratory distress syndrome, ARDS), as well as aggravation of respiratory tract hypersensitivity due to therapy with other medical drugs;
pneumoconiosis of any type, etiology or pathogenesis, or pneumoconiosis from the group of aluminosis or aluminum pneumoconiosis, anthracosis (asthma), asbestosis or asbestos pneumoconiosis, chalicosis or lime pneumoconiosis, ptilosis caused by inhalation of ostrich feather dust, siderosis caused by inhalation of iron particles, silicosis or Potter's asthma, byssinosis or cotton pneumoconiosis, as well as talc dust pneumoconiosis;
bronchitis of any type, etiology or pathogenesis, or bronchitis from the group of acute bronchitis, acute laryngotracheal bronchitis, bronchitis induced by peanuts, bronchial catarrh, croupous bronchitis, unproductive bronchitis, infectious asthma bronchitis, bronchitis with sputum, staphylococcal or streptococcal bronchitis; as well as vesicular bronchitis;
bronchiectasia of any type, etiology or pathogenesis, or bronchiectasia from the group of cylindrical bronchiectasia, saccular bronchiectasia, spindle bronchiectasia, bronchiole dilatation, cystic bronchiectasia, unproductive bronchiectasia, as well as follicular bronchiectasia;
seasonal allergic rhinitis, perennial allergic rhinitis, or sinusitis of any type, etiology or pathogenesis, or sinusitis from the group of purulent or non-purulent sinusitis, acute or chronic sinusitis, ethmoiditis, frontal sinusitis, maxillary sinusitis, or sphenoiditis;
rheumatoid arthritis of any type, etiology or pathogenesis, or rheumatoid arthritis from the group of acute arthritis, acute gouty arthritis, primary chronic polyarthritis, osteoarthrosis, infectious arthritis, Lyme arthritis, progredient arthritis, psoriatic arthritis, as well as spondylarthritis;
gout as well as fever associated with inflammation, or pain associated with inflammation;
eosinophile-related pathologic disorders of any type, etiology or pathogenesis, or eosinophile-related pathologic disorders from the group of eosinophilia, eosinophilic pulmonary infiltrate, Löffler's syndrome, chronic eosinophilic pneumonia, tropical pulmonary eosinophilia, bronchopneumonic aspergillosis, aspergilloma, eosinophilic granuloma, allergic granulomatous angiitis or Churg-Strauss syndrome, polyarteritis nodosa (PAN), as well as systemic necrotizing vasculitis;
atopic dermatitis, allergic dermatitis, or allergic or atopic eczema;
urticaria of any type, etiology or pathogenesis, or urticaria from the group of immune-related urticaria, complement-related urticaria, urticaria induced by material causing urticaria, urticaria induced by physical stimuli, urticaria induced by stress, idiopathic urticaria, acute urticaria, chronic urticaria, angioneurotic edema, Urticaria cholinergica, cold urticaria in its autosomal-dominant or acquired form, contact urticaria, Urticaria giantean as well as papuloid urticaria;
conjunctivitis of any type, etiology or pathogenesis, or conjunctivitis from the group of actinic conjunctivitis, acute catarrhal conjunctivitis, acute contagious conjunctivitis, allergic conjunctivitis, atopic conjunctivitis, chronic catarrhal conjunctivitis, purulent conjunctivitis, as well as spring conjunctivitis;
uveitis of any type, etiology or pathogenesis, or uveitis from the group of inflammation of the whole uvea or a part thereof, Uveitis anterior, iritis, cyclitis, iridocyclitis, granulomatous uveitis, non-granulomatous uveitis, phacoanti-genic uveitis, Uveitis posterior, choroiditis, as well as chorioretinitis;
psoriasis;
multiple sclerosis of any type, etiology or pathogenesis, or multiple sclerosis from the group of primary progredient multiple sclerosis, as well as multiple sclerosis with episodic course and tendency of remission;
autoimmune/inflammatory diseases of any type, etiology or pathogenesis, or autoimmune/inflammatory diseases from the group of autoimmune-hematological disorders, hemolytic anemia, aplastic anemia, aregenerative anemia, idiopathic thrombocytopenic purpura, systemic lupus erythematosus, polychondritis, scleroderma, Wegener's granulomatosis, photopathy, chronically active hepatitis, Myasthenia gravis, Stevens-Johnson syndrome, idiopathic sprue, autoimmune irritable colon disease, ulcerous colitis, Crohn's disease, endocrine opthalmopathy, Basedow's disease, sarcoidosis, alveolitis, chronic hypersensitive pneumonitis, primary biliary cirrhosis, insulin deficiency diabetes or type 1 pancreatic mellitus, Uveitis anterior, granulomatous uveitis or Uveitis posterior, dry keratoconjunctivitis, epidemic keratoconjunctivitis (diffuse), interstitial pulmonary fibrosis, pulmonary cirrhosis, mucoviscidosis, psoriatic arthritis, glomerulonephritis with and without nephrosis, acute glomerulonephritis, idiopathic nephrosis, minimal-change nephropathy, inflammatory/hyperproliferative dermal diseases, psoriasis, atopic dermatitis, contact dermatitis, allergic contact dermatitis, familial benign pemphigus, Pemphigus erythematosus, Pemphigus foliaceus as well as Pemphigus vulgaris;
preventing allograft rejection after organ transplantation, irritable intestine (inflammatory bowel disease, IBD) of any type, etiology or pathogenesis, or irritable intestine from the group of ulcerous colitis (UC), collagenous colitis, Colitis polyposa, transmural colitis, as well as Crohn's disease (CD);
septic shock of any type, etiology or pathogenesis, or septic shock from the group of renal failure, acute renal failure, cachexia, malaria cachexia, hypophyseal cachexia, uremic cachexia, cardiac cachexia, Cachexia suprarenalis or Addison's disease, carcinomatous cachexia, as well as cachexia due to infection with human immunodeficiency virus (HIV); liver damage;
pulmonary hypertension, as well as pulmonary hypertension induced by oxygen deficiency;
bone rarefaction diseases, primary osteoporosis and secondary osteoporosis;
any type of pathologic disorders of the central nervous system, etiology or pathogenesis, or pathologic disorders of the central nervous system from the group of depression, Parkinson's disease, learning and memory disorders, tardive dyskinesia, drug addiction, arteriosclerotic dementia, as well as dementia as an accompanying symptom of Huntington's disease, Wilson's disease, agitated paralysis, as well as thalamus atrophy;
infections, especially viral infections, such viruses increasing the production of TNF-α in their host or being sensitive to TNF-α upregulation in their host, thereby impairing their replication or other important activities, including viruses from the group of HIV-1, HIV-2 and HIV-3, cytomegalovirus, CMV; influenza, adenovirus and herpes viruses, including Herpes zoster and Herpes simplex; yeast and fungous infections, such yeasts and fungi being sensitive to upregulation by TNF-α or inducing TNF-α production in their host, preferably fungous meningitis, especially in case of simultaneous administration with other drugs of choice for the treatment of systemic yeast and fungous infections, including polymycins, preferably polymycin B, imidazoles, preferably clotrimazol, econazol, miconazol and/or ketoconazol, triazoles, preferably fluconazol and/or itranazol, as well as amphotericins, preferably amphotericin B and/or liposomal amphotericin B.

The invention also relates to a method for the modification, especially inhibition, of an AKAP-PKA interaction, comprising the steps of:
(a) providing the decoupler of the invention or a recognition molecule targeted thereto, and
(b) contacting at least one product according to (a) with a cell, cell culture, tissue and/or target organism.

In a preferred embodiment of the invention, the above method is characterized in that modification is effected on a regulatory RII subunit of PKA, the RII subunits preferably being RIIalpha and/or RIIbeta subunits.

The invention also relates to a kit comprising the products of the invention, preferably the decouplers and the recognition molecules targeted thereto, and/or a pharmaceutical composition according to the invention, optionally together with information—e.g. an instruction leaflet or an internet address referring to homepages including further information, etc.—concerning handling or combining the contents of the kit. For example, the information concerning handling the kit may comprise a therapeutic regimen for the above-mentioned diseases, particularly the preferred diseases. Also, the information may comprise information referring to the use of the products of the invention in diagnosing diseases associated with AKAP-PKA interaction or decoupling thereof. The kit according to the invention may also be used in basic research. In basic research, the kit can preferably be used to detect whether a metabolic phenomenon is associated with interaction or absent interaction of AKAP and PKA. More specifically, the kit according to the invention allows to determine which subunits of AKAP and/or PKA are responsible for interaction of the above two molecules or failure of such interaction to take place.

In an advantageous embodiment the products according to the invention may comprise peptides, vectors, nucleic acids, amino acids, carbohydrates or lipids. For example, it may be preferred to couple the products to a fatty residue, so that the membrane permeability will be changed. By comparison with substances binding PKA with different affinity, it will also be possible to make quantitative statements defining to what extent PKA-AKAP interaction is necessary to ensure the progress of a physiological process. In particular, the kits according to the invention can be used to study the progress of such a physiological process. Advantageously, the molecules of the invention can be selected in such a way that they bind the RII subunits of PKA more strongly than the typical PKA binding domains of AKAP, preferably AKAP18, particularly of AKAP18delta. Advantageously, selected molecules of the invention are specific to RIIalpha or RIIbeta or to particular RI subunits, so that the kit can be used e.g. to obtain highly detailed insight into the interaction of these molecules. More specifically, decoupling of one or another regulatory subunit of PKA from AKAP proteins may furnish information as to which PKA, type IIalpha or type IIbeta or type I, is involved in each process to be investigated.

The invention also relates to a method for the production of pharmaceutical agents, which method comprises the following steps:
(a) providing a decoupler according to the invention, preferably in the form of a lead structure,
(b) chemical modification of the lead structure, preferably by means of combined and/or structure-based drug design, thereby obtaining substances, and optionally
(c) testing the substances for their capability of influencing the AKAP-PKA interaction, and optionally
(d) selecting suitable substances as pharmaceutical agents.

In a preferred embodiment of the invention, the above method also comprises formulating the tested substances into a pharmaceutically acceptable form.

The invention also relates to the processed product directly obtained by the above method.

Without intending to be limiting, the invention will be explained in more detail with reference to the examples. The inhibitors or decouplers of the invention modulate the interaction of AKAP and PKA. The invention will be described in more detail below with reference to a few selected examples.

### 1. Materials and Methods

Unless otherwise stated, the reagents and chemicals used were purchased from Merck (Darmstadt), Sigma (Deisenhofen) or Carl Roth (Karlsruhe).

### 1.1 Media and Buffers

Luria-Bertani (LB) medium
10 g/l tryptone

### 10 g/l NaC₁₋₅

5 g/l yeast extract
pH 7.0

The solution was autoclaved after intense stirring.

### LB Agar

LB medium was added with 15 g/l agar and subsequently autoclaved.

To prepare agar plates, the LB agar was heated in a microwave oven until all solid particles had dissolved. Following cooling to 40° C., the required antibiotic was added and the solution cast into plates.

### Ampicillin

Ampicillin is a penicillin derivative preventing cell wall synthesis in proliferating bacteria. It was stored in the form of stock solution (100 mg/ml) at −20° C. and diluted to a concentration of 100 μg/ml in LB agar or LB medium shortly before use.

20× Tris/acetic acid/ethylenediamine Tetraacetate (EDTA) Buffer (TAE)
242 g of tris(hydroxymethyl)aminomethane (Tris) in 0.5 l of H₂O

### 40 ml of 0.5 M EDTA, pH 8

22.8 ml of acetic acid

### ad 1 l of H₂O

### Ethidium Bromide Solution

1 g of ethidium bromide

100 ml of H₂O

### 6× Deoxyribonucleic Acid (DNA) Test Buffer

250 mg of bromophenol blue 250 mg of Xylencyanol

### 33 ml of 150 mM Tris-HCl, pH 7.6

60 ml of glycerol

### 7 ml of H₂β

### Isopropyl-β-D-thiogalactoside (IPTG) Solution

### 1.79 g of IPTG

### 50 ml of H₂O

### Phosphate-Buffered Saline (PBS)

### 28.5 g of Na₂HPO₄×₂H₂O

### 5.5 g of NaH₂PO₄×H₂O

### 164 g of NaCl

### ad 1 l H₂O

### Mix of Protease Inhibitors

1.4 μg/μl trasylol (aprotinin) 0.5 mM benzamidine
3.2 μg/μl soy bean trypsin inhibitor (STI)
Phenylmethylsulfonyl fluoride (PMSF) Solution (40 mM)

### 14 mg of PMSF

2 ml of ethanol
Dithiothreitol (DTT) stock solution (0.5 M)

### 771 mg of DTT

### 10 ml of H₂O

### Lysis Buffer

### PBS

Mix of protease inhibitors (1:125)
PMSF solution (1:80)
DTT stock solution (1:100)

### Lysozyme Solution

10 mg of lysozyme

### 10 ml of H₂O

### Glutathione Elution Buffer

40 mM reduced glutathione

### 200 mM NaCl

### 0.2% Tween 20

### 100 mM Tris-HCl, pH 8.5

Sodium dodecylsulfate (SDS) Sample Buffer

### 100 mM Tris-HCl, pH 6.8

### 4% SDS

20% glycerol

### 10% β-Mercaptoethanol

0.02% bromophenol blue

### 30 mM DTT

### Separating Gel (10%)

the amounts specified are sufficient for two gels:
3.75 ml of acrylamide 30% with bisacrylamide 0.8%

### 5.625 ml of Tris-HCl 0.75 M, pH 8.8

### 56.5 μl of SDS 20%

### 2.5 ml of H₂O

79 μl of ammonium peroxodisulfate (APS) 10%
5.65 μl of N,N,N′,N′-tetramethylethylenediamine (TEMED)

### Collecting Gel

the amounts specified are sufficient for two gels:
835 μl of acrylamide 30% with bisacrylamide 0.8%

### 625 μl of Tris-HCl 0.625 M, pH 6.8

### 25 μl of SDS 20%

### 3.5 ml of H₂O

### 25 μl of APS 10%

### 5 μl of TEMED

SDS polyacrylamide gel electrophoresis (PAGE) migration buffer
250 mM glycine

### 25 mM Tris

### 0.1% SDS

### Coomassie Staining Solution

2 g of coomassie brilliant blue G 250
75 ml of acetic acid
500 ml of methanol

### ad 1 l H₂O

### Destaining Solution

75 ml of acetic acid
100 ml of ethanol
ad 1 lH₂O

### Semi-Dry Transfer Buffer

### 25 mM Tris

190 mM glycine
20% methanol

### Tris-Buffered Saline (TBS)

### 10 mM Tris-HCl, pH 8.0

### 150 mM NaCl

### TBS-Tween 20 (TBST)

As in TBS, with 0.01% Tween 20

### Ponceau S Staining Solution

### 2 g of Ponceau S

30 g of trichloroacetic acid
30 g of sulfosalicylic acid

### 100 ml of H₂O

### Blotto

50 g/l skim milk powder in TBST

### Bradford's Reagent

100 mg of coomassie brilliant blue G 250
50 ml of ethanol, non-denatured

### 800 ml of H₂O

Allow solution to stand overnight, subsequently add
100 ml of phosphoric acid
ad 1 lH₂O
The solution was agitated and filtrated.

### Binding Buffer

80 μl of protease inhibitor mix
125 μl of PMSF solution
20 μl of DTT stock solution 0.5 M

### ad 10 ml PBS

### Blocking Solution

150 mg of skim milk powder
100 μl of 0.5 M DTT stock solution

### 0.05% Tween 20

625 μl of PMSF solution
400 μl of protease inhibitor mix

### ad 50 ml PBS

### PBST

### 0.05% Tween 20 in PBS

1.2 Transformation of Competent Bacteria with Plasmid DNA

For transformation, competent E. coli cells of the strain BL21(DE3) were produced according to the method of Cohen and Wang.

The bacteria stored at −80° C. were thawed twice on ice, and 2 ng of the plasmid DNA to be transformed was added to 50 μl of bacteria suspension. After incubating for 30 min on ice, the cells were exposed to a thermal shock of 42° C. for 45 s in order to receive the plasmid DNA. Thereafter, 250 μl of LB medium preheated to 37° C. was added immediately, and the cells were agitated for 1 h at 37° C., allowing them to express the antibiotic resistance encoded by the plasmid. Thereafter, 30 μl of cell suspension was removed and plated on LB agar containing antibiotic agent; the remainder was centrifuged at 8000×g for 1 min, resuspended in 100 μl of medium and plated as well. The agar plates were incubated at 37° C. overnight. On the next day, the colonies were counted to quantify the transformation efficiency.

### 1.3 Plasmid DNA Preparation

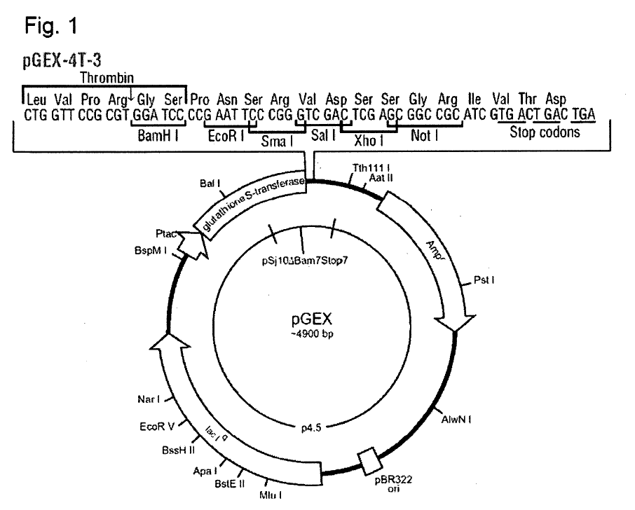

For expression of GST fusion proteins, the pGEX-4T3 plasmid (FIG. 1) including the cloned protein-coding sequence was used. Such a vector can be used to introduce foreign DNA in bacterial cells, and the plasmid DNA can be re-isolated from the cells e.g. for control purposes.

To isolate 5-10 μg of plasmid DNA from E. coli, 2-3 ml of LB medium was inoculated with the corresponding antibiotic with single colonies from agar plates. The cultures were incubated with agitation at 37° C. overnight.

The cells were centrifuged at 13,000×g, and the supernatant was discarded completely, if possible.

Plasmid isolation from the bacteria sediments was carried out according to the plasmid mini-preparation protocol of the QIAprep Spin Miniprep Kit (Qiagen GmbH, Hilden).

To remove proteins and protein-associated chromosomal DNA, the cells were lysed in alkaline lysis buffer. The soluble plasmid DNA was bound on silica gel (in a column) in the presence of a highly concentrated salt solution, washed, and finally eluted using a salt solution of low concentration. The isolated plasmid DNA was analyzed using restriction endonuclease digestion and subsequent agarose gel electrophoresis.

### 1.4 DNA Restriction

Restriction endonucleases such as EcoRI (isolated from E. coli) cut DNA at specific sites defined by the sequence. The enzymes recognize such sequences, bind thereto, and cut the DNA by hydrolysis. Microorganisms such as E. coli possess restriction endonucleases to degrade foreign DNA. Differentiation between autologous and foreign DNA is made possible by varying methylation patterns of the DNA.

The endonucleases purchased from different manufacturers (New England BioLabs (NEB), Beverly, USA; Fermentas GmbH, St. Leon-Rot; Invitrogen GmbH, Karlsruhe) were used according to the manufacturers' instructions.

In general, DNA restrictions (treatment with restriction endonucleases) were performed at 37° C. for 1 h. A typical reaction batch included 5 μg of DNA, 1 μl of 10× enzyme buffer and 0.5 μl of enzyme solution and was filled up to a final volume of 10 μl with sterile water.

Following incubation, the complete reaction batch was analyzed using agarose gel electrophoresis.

### 1.5 Agarose Gel Electrophoresis

In electrophoresis, molecules are separated according to size and electric charge in a gel by applying a voltage. 1% agarose gels were used. For preparation, 1% solid agarose was placed in TAE buffer and dissolved by boiling in a microwave oven. The clear solution was cooled to 45° C., and 5 μl of ethidium bromide (EtBr) solution was added per 100 ml of agarose solution.

EtBr intercalates between the bases of nucleic acids, allowing detection thereof under UV light. The EtBr fluorescence is intensified by delocalized 1-electron systems of purine and pyrimidine bases of DNA, which have EtBr intercalated therebetween.

The liquid gel was subsequently poured into a plastic mold, where it was allowed to cool and polymerize.

The DNA samples to be investigated were added with sample buffer, TAE, glycerol and bromophenol blue. Glycerol increases the density of the solution, so that the latter could reach the gel pockets easier and was held there. Bromophenol blue allows visualization of the sample.

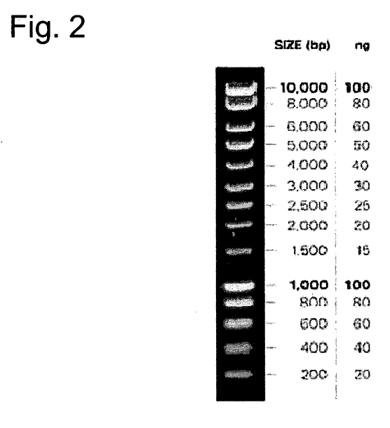

To determine the size of the DNA fragments in the sample, 5 μl of a DNA molecular weight standard was co-analyzed with DNA fragments of a defined size in one lane per gel (Hyper-Ladder I, Bioline GmbH, Luckenwalde; FIG. 2).

Electrophoresis was performed at a voltage of 120 V for 30 min. Thereafter, the gels were photographed with a LumiImager F1 from Boehringer Mannheim and analyzed with the Lumi Analyst 3.0 Software included.

### 1.6 Expression and Purification of GST Fusion Proteins

150 ml of LB medium containing ampicillin was inoculated with E. coli cells of a transformed clone from an agar plate. The culture solution was incubated at 30° C. overnight (ON). (At 37° C., cell growth is excessively rapid, so that the culture is already beyond the logarithmic growth phase on the next day; furthermore, the elevated incubation temperature results in stronger basal expression of the fusion protein. This may impede purification as a result of formation of insoluble inclusion bodies.)

On the next day, 20 ml of the overnight culture was transferred into 500 ml of fresh LB medium containing ampicillin. During incubation at 37° C., growth of the bacteria was monitored by measuring the optical density (OD) at 600 nm. After reaching an OD₆₀₀ of about 0.6—the bacteria now being in the logarithmic growth phase where protein expression is at maximum—expression of the glutathione S transferase (GST) fusion protein was induced by adding IPTG at a final concentration of 1.5 mM. IPTG is a synthetic inductor of the lac operon, binding to the repressor and thus inhibiting binding thereof to DNA. Following incubation at 37° C. for 30 min, the bacteria were sedimented by centrifugation for 10 min at 5000×g and 4° C.

From this point on, all the following purification steps were carried out on ice in order to keep the activity of proteases low. The sediment was resuspended in 30 ml of cold lysis buffer, and DTT was added at a final concentration of 5 mM. The cell suspension was mechanically lysed three times using a French Pressure Cell Press (“French press”). During this treatment, the bacterial cell walls were destroyed as a result of the high pressure, thus facilitating subsequent lysis of the plasma membranes by a detergent.

Triton X-100 at a final concentration of 1% was used as detergent. The suspension was slightly agitated at 4° C. for 30 min to dissolve the plasma membranes and release the cytosolic proteins into the solution.

The lysate thus obtained was centrifuged at 17,000×g and 4° C. for 10 min to remove cell debris. The supernatant contained the GST fusion proteins and—to purify the latter—was incubated with 600 μl of Glutathione Sepharose 4B (50% suspension in lysis buffer; Amersham GmbH & Co. KG, Brunswick) for 30 min at room temperature with slight agitation. In vivo, the GST enzyme plays an important role in detoxification reactions, e.g. in the cytosol of hepatocytes, catalyzing the reaction of glutathione with various electrophilic hydrophobic substrates. In protein purification, the GST fused with the desired protein binds with high affinity and high specificity to glutathione coupled to Sepharose beads. Sepharose is a bead-shaped agarose-derived base material crosslinked to form a three-dimensional network. Owing to their large volume, the beads and anything bound thereto can be sedimented by centrifugation at 500×g for 5 min.

Alternatively, GST fusion proteins can also be purified using affinity chromatography on a column filled with Glutathione Sepharose. This operation usually results in higher purity, but also lower yield of proteins.

The Sepharose sediment including the GST fusion proteins was subsequently washed three times, which was done by resuspending in 3 ml of lysis buffer and recentrifuging each time.

To elute the purified proteins from the Glutathione Sepharose, the pellet was added with 300 μl of elution buffer and agitated at room temperature for 10 min. The elution buffer included free glutathione in excess, thus competitively displacing the Glutathione Sepharose from the GST fusion protein. Following centrifugation at 500×g for 5 min, the supernatant containing the fusion protein was collected by careful pipetting, added with the same volume of glycerol, aliquoted, and stored at −20° C. Glycerol prevents formation of ice crystals resulting in degradation of the proteins (particularly after several freeze/thaw cycles).

To allow monitoring of the purification process, samples were taken at various stages of the procedure and analyzed using SDS PAGE (see below): before and after inducing protein synthesis with IPTG, before and after centrifugation of the cell lysate, before and after adding Glutathione Sepharose, and after washing the Sepharose sediment.

### 1.7 SDS PAGE

The method used to analyze the purity and molecular weight of proteins is discontinuous sodium dodecylsulfate-polyacrylamide gel electrophoresis (SDS PAGE) according to Laemmli.

To detect the molecular weight, it is necessary to eliminate the influence of charge and tertiary structure on the migration rate of the proteins in the gel, so that the (logarithmic) mobility of the proteins depends on their molecular weight alone.

This situation can virtually be achieved by denaturing the native proteins with the SDS detergent which results in dissociation of oligomeric proteins into subunits thereof. The negative charges of the sulfonic acid groups of SDS overlay the inherent charges of the individual amino acid side chains, thus providing for uniform negative charge of the proteins.

Disulfide bridges are reduced and cleaved by DTT and β-mercaptoethanol present in the sample buffer in addition to SDS. The denatured proteins bind SDS in accordance with their size, i.e., length of the amino acid chain.

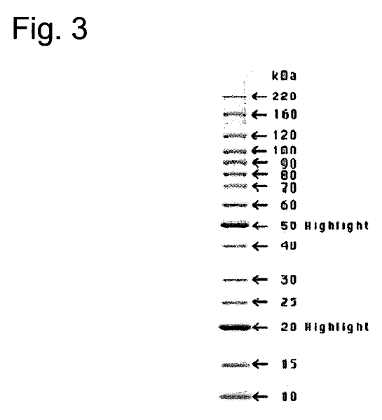

As in agarose gel electrophoresis of nucleic acids, a molecular weight standard consisting of proteins having known molecular weights is co-applied to the gel. To this end, a BenchMark Protein Ladder molecular weight standard was used (FIG. 3; Invitrogen GmbH, Karlsruhe).

The SDS PAGE is referred to as discontinuous because the gel consists of two parts, namely, a collection gel and a separation gel which differ in their pore size and pH value. Such a discontinuous system provides sharper bands compared to a continuous system, thus allowing more precise determination of protein size and molecular weight.

Sepharose beads with bound proteins can be used directly in SDS PAGE because the SDS sample buffer has high reducing power, resulting in elution of the proteins from the beads. The separation gel was produced using the reagents specified in section 2.1. Polymerization started after addition of TEMED by pipetting, and 3.75 ml of solution was pipetted between glass plates clamped in a special plastic holder.

The gel was subsequently covered with 2-propanol to eliminate air bubbles and keep air away from the gel (oxygen prevents the polymerization reaction). The 2-propanol was removed after completed polymerization, and the collection gel was prepared (see section 1.1). After covering the separation gel with a layer of collection gel, the ridge forming the sample pockets was placed in the gel solution.

The protein samples were denatured by boiling in sample buffer at 95° C. for 5 min. The samples were briefly centrifuged and transferred into the gel pockets using a Hamilton syringe.

After electrophoresis at 90 V for about 15 min, the proteins reached the separation gel, and the voltage was increased to 120 V for 1.5 h.

Following completion of the electrophoresis, the separated proteins in the gel were either stained with coomassie brilliant blue G 250 solution or further investigated using Western blot analysis (see below).

For coomassie staining, the gel was agitated for 30-60 min in coomassie staining solution and subsequently in destaining solution until excess dye was removed and the contrast between unstained gel and stained proteins was sufficiently strong. Finally, a photograph was taken using the Bio-Rad ChemiDoc EQ and the Quantity One software included.

### 1.8 Western Blot

In Western blotting, the proteins—following separation by means of SDS PAGE—are transferred on a nitrocellulose or polyvinylidene fluoride (PVDF) membrane by means of an electric voltage and detected using specific antibodies (Ab). After binding of the Abs, excess Abs are washed off, and the membrane is incubated with a secondary Ab which is labelled and specifically recognizes the primary Ab. The label may consist of radioactive isotopes or an enzyme catalyzing a dye-forming reaction.

First of all, an Immobilon P-PVDF membrane was cut to the size of the gel and wetted for 15 s with ethanol and subsequently for at least 5 min with Semidry transfer buffer. Whatman filter papers were also cut to proper size and wetted in transfer buffer.

Protein transfer from the gel onto the membrane was carried out using a BioRad TransBlot SD Semi Dry Transfer Cell which had 2 filters, a membrane, a gel and another 2 filters layered thereon. Air bubbles were removed, because they impair protein transfer. After pipetting 4 ml of transfer buffer on the stack, the transfer cell was closed, and the proteins were transferred onto the membrane at 10 V for 1 h.

Subsequently, the proteins on the membrane were stained with Ponceau S solution. To this end, the membrane was washed with water and agitated in Ponceau S solution at room temperature for 20 min. The bands of the molecular weight standard were traced with a pencil, and the membrane was wrapped in transparent film in order to take a photograph using the LumiImager F1 (exposure 7 s, top illumination).

Prior to Ab incubation, free binding sites on the membrane must be blocked because otherwise, large quantities of Ab may attach thereto in a non-specific fashion, interfering with specific signals. Saturation of the membrane was achieved by agitating in Blotto for 1 h.

The primary A18delta3 Ab was already available to the team. It had been produced by immunizing rabbits to a peptide whose sequence was identical with the amino acids 60-76 of the AKAP18delta sequence. The antiserums thus obtained were purified by affinity chromatographic via the peptides used for immunization, coupled to Thiopropyl Sepharose 6B (Amersham GmbH & Co. KG, Brunswick). The A18delta3 Ab binds both AKAP18delta and -gamma.

The Ab solution was diluted 1:500 in Blotto. To reduce the amount of Abs required, the membranes were agitated in incubation bags with 3 ml of Ab solution free of air bubbles for 2 h at room temperature or at 4° C. overnight.

After washing three times with TBST for 5 min, the membranes were agitated for 1 h in a 1:1000 dilution of secondary Ab (anti-rabbit F(ab)₂ fragments) in Blotto. The secondary Ab was coupled to horseradish peroxidase (POD; Dianova, Hamburg).

The membranes were washed three times with TBST for 10 min; for detection, 4 ml of solutions 1 and 2 of the LumiLight Western Blotting Substrate (Roche Diagnostics GmbH, Mannheim) were added to the membrane, and this was agitated for 5 min at room temperature. The detection solution includes luminol which is oxidized by the Ab-coupled POD with generation of light (chemiluminescence). A photograph of the membrane wrapped in transparent film was taken, adjusting “chemiluminescence” (exposure 1 min) on the LumiImager F1.

### 1.9 Bradford Determination of Protein Concentration

The Bradford method is best suited to determine the protein concentration in the Glutathione Sepharose eluates, because the chromogen being used (coomassie brilliant blue G 250) does not bind to excess glutathione also present in the eluate, but particularly to cationic and hydrophobic amino acid side chains.

For measurement, 50 μl of the protein-containing sample (diluted eluate) was added to 50 μl of 2 M NaOH and incubated at 60° C. for 10 min. Thereafter, 1 ml of Bradford reagent was added, the sample was transferred into a cuvette and, the absorption at 595 nm was determined.

The protein concentration was determined by comparison with a calibration series from various ovalbumin dilutions.

### 1.10 Enzyme-Linked Immunosorbent Assay (ELISA)

ELISA is a method wherein an antigen is detected with a specific antibody. Detection proceeds via an enzyme linked covalently with one of the binding partners, which enzyme catalyzes chromogen conversion. This results in quantitative generation of e.g. a dye or chemiluminescence (as described in the Western blot).

Because one of the two binding partners is immobilized, removal of unbound antibodies or antigens is very easy. Detection proceeds via observing the intensity (I) of chemiluminescence using a photomultiplier in a special reading device (ELISA reader). The output of the measured values is in relative light units (RLU).

In the present case, the point was to investigate whether binding of one protein (PKA) to another (AKAP18delta-GST) would be inhibited by the presence of small organic molecules. A sandwich ELISA was therefore developed wherein the RIIalpha PKA subunit, to which AKAP proteins bind, was bound on 384-well microtiter plates (MTP).

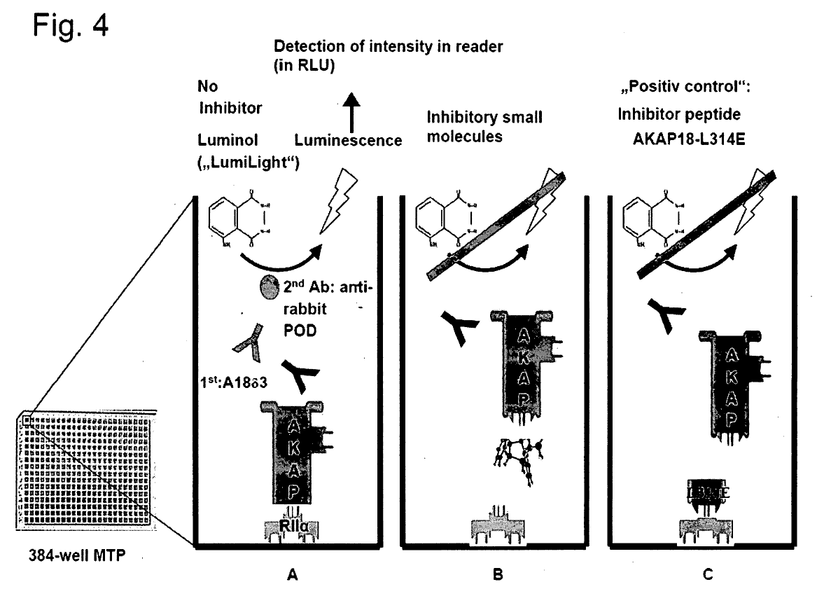

White MTPs made of polystyrene with high protein binding capacity were used (384-Well White Flat Bottom Polystyrene High Bind Microplate, Product No. 3703, Corning GmbH, Wiesbaden). The white color improves detection by reflection of the chemiluminescence being formed and simultaneously prevents illumination of neighboring wells. Free binding sites were blocked after binding (see below), and the second GST-AKAP18delta protein was added to inhibitory peptides (see below) together with the potential low-molecular weight inhibitors as control. Subsequently, bound GST-AKAP18delta was quantified via antibodies and chemiluminescence. The principle of ELISA is illustrated in FIG. 4.

PBS (phosphate-buffered saline, pH 7.4) was used as buffer system to maintain a physiological pH value for the proteins used. The buffer was added with a mix of protease inhibitors and PMSF as non-specific protease inhibitor to delay protein degradation. In addition, DTT was employed to protect the proteins from oxidation.

The blocking solution additionally included 0.3% skim milk powder to saturate MTP non-specific binding sites with milk proteins included therein.

To establish an ELISA as described in more detail in the section dealing with the results, further antibodies in addition to those specified above and described in section 1.8 (Western blot) were required in the detection of PKA-RIIalpha: the commercially available mouse PKA_{RIIalpha} antibody (BD Biosciences, San Jose, USA) and an anti-mouse POD Ab (Dianova, Hamburg).

### 1.11 Calculation of Binding Curves and IC₅₀ Values

Part of the data obtained from the experiments were evaluated using GraphPad Prism (GraphPad Software, San Diego, USA).

To adapt the binding curves to the measured values (see results), the one-site binding model was used as a basis, which model applies to binding of GST-AKAP18delta to RIIalpha. The corresponding formula is

$$Y = \frac{{B}_{max} \cdot X}{{K}_{d} + X}$$

The measured values for IC₅₀ calculation were adapted to the one-site competition model. It was established that the bottom asymptote should be greater than 0.0 to rule out faulty adaptations with negative binding values. The formula used is

$$Y = \frac{Bottom + ( Top - Bottom )}{1 + {10}^{X - log / {C}_{50}}}$$

### 1.12 Substance Library Screening

The FMP20000 substance library was used for screening, i.e., systematic, partially automated search for potential inhibitors of AKAP18delta-RIIalpha binding. The library included 20,064 different, commercially available substances in 10 mM stock solutions in DMSO on 57 MTPs, each having 384 wells (ChemDiv, San Diego, USA). The substances of the library had been selected according to specific criteria which also apply to previously known pharmacological active substances. Said criteria also include Lipinski's Rule (cf. discussion). To prepare an MTP for screening, each of 380 wells was filled with 20 μl of RIIalpha solution with a concentration of 0.75 ng/μl (in binding buffer), using an electronically controlled 24-channel pipette (Eppendorf). 10 μl of AKAP18delta-GST (8 ng/l) was pipetted into each of the remaining 4 wells so as to have a positive control of detection (cf. MTP occupancy in Table 1.1 and pipetting scheme in Table 1.2). Following centrifugation of the MTPs (1 min at 2500×g), the MTPs were incubated at room temperature for at least 1 h. After removing the protein solution by tapping on a paper stack, non-specific binding sites were blocked with 100 μl of blocking solution/well for at least 1 h at room temperature.

Table 1.1: Occupancy of an MTP with control reactions (columns 1 and 24) and low-molecular weight substances (2-23). See also pipetting scheme in Table 1.2.
$

The blocking solution was removed using an automatic Power Washer 384 (Tecan), and all wells were washed with TBST. After completion of the washing operation, solution residues possibly still present were tapped off on a paper stack.

In the next step, the MTPs were prepared for automated pipetting of the low-molecular weight substances. Each well was supplied with 10 μl of blocking solution or controls pipetted in accordance with the pipetting scheme (Table 1.2). Reactions with no inhibitors, but with increasing amounts of AKAP18delta-GST (0, 80, 160 ng/well), and with the inhibitory L314E peptide and the 18d-PP control peptide (25 nM and 1 μM final concentration) were used as controls. The plates were briefly centrifuged.

At the same time, the library MTPs were prepared for the pipetting operation as follows: thawing (30 min at 37° C.) was followed by brief centrifugation to accumulate the solutions on the plate bottom. To dissolve solid particles possibly present, the plates were immersed in an ultrasonic bath for 1 min. Water adhering to the plates was evaporated by treatment in an Eppendorf Concentrator 5301 at 30° C. for 5 min, and the protective film was carefully removed.

Using a Sciclone ALH 3000 Workstation (Caliper LifeSciences, Hopkinton, USA), 0.5 μl of each potential low-molecular weight inhibitor (10 mM stock solutions in dimethyl sulfoxide (DMSO), final concentration: 244 μM) was transferred from the library MTPs to the screening MTPs. Each of the control columns 1 and 24 received 0.5 μl of DMSO, so that the controls were added with the same amount of DMSO as the other samples.

Following centrifugation of the screening MTPs, AKAP18delta-GST (8 ng/μl in blocking solution) was added in accordance with the pipetting scheme (Table 2.2), centrifuged, and incubated for at least 30 min at room temperature.

After removal of excess protein by washing with PBST in the Power Washer, the amount of AKAP18delta-GST bound in the presence of a potential inhibitor was detected using 20 μl of A18delta3 (1:1000 in blocking solution, at least 1 h at room temperature or at 4° C. overnight) and 20 μl of anti-rabbit POD Ab (1:3000 in blocking solution, at least 15 min at room temperature) per well. Washing with PBST using the Power Washer was effected between each single pipetting step. Quantification was carried out by pipetting 20 μl of LumiLight Western blotting substrate (Roche) per well and incubating for 5 min at room temperature. The chemiluminescence generated was detected using a Tecan Genios Pro MTP reader and the Magellan 5.02 software included (chemiluminescence endpoint measurement, integration time: 10 ms/well; Tecan Deutschland GmbH, Crailsheim).

Table 1.2: Pipetting scheme for a screening MTP. The control reactions are in columns 1 and 24, and in 2-23 the reactions with low-molecular weight substances.

*In 1E/1F/24KJ24L: After pipetting the small molecules, the blocking buffer, 10 μl, was replaced by 20 μl of GST-AKAP18delta. $

### 2. Results

### 2.1 Purification of Recombinant AKAP18Delta

2.1.1 The Plasmid Encoding the AKAP18delta-GST Fusion Protein

The pGEX-4T3 vector suitable for expression of GST fusion proteins (FIG. 1) and including the cloned AKAP18delta-encoding sequence had already been available to the team. The AKAP18delta cDNA fragment comprising 1059 base pairs was situated at the 3′ end of the GST-encoding sequence, so that GST in the expressed protein was situated at the N-terminal end of AKAP18delta. The correctness of the sequence had been confirmed by sequencing a short time before this work was initiated.

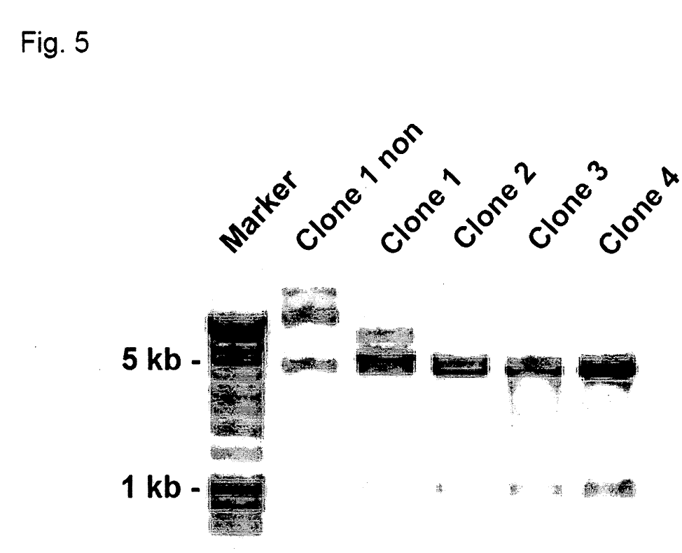

For expression, the GST-AKAP18delta construct was trans-formed into competent BL21 cells. To check the transformation, the plasmid DNA from four of the clones obtained was isolated, and a DNA restriction was carried out using the restriction endonucleases EcoRI and XhoI. Because these enzymes were also used for cloning, fragments having the size of the AKAP18delta insert (1.0 kb) and of the linearized pGex vector (4.9 kb) were detected in agarose gel electrophoresis (FIG. 5) as expected.

2.1.2 Optimizing the Lysis of Bacteria Expressing GST-AKAP18delta

By incubating in IPTG-containing medium, it was possible to make bacteria transformed with the pGEX-AKAP18delta vector express the recombinant protein. Following subsequent lysis of the bacterial cells, the GST fusion protein released into the solution was bound to Glutathione Sepharose, washed, and finally eluted with excess glutathione.

To achieve a protein yield as high as possible, various parameters of GST-AKAP18delta purification were varied, starting from a standard method. The purification procedure established in this way is described in section 1.6.

A size of 75 kDa was expected for the purified recombinant GST-AKAP18delta and of 25 kDa for GST alone, which had been purified for control purposes.

Following transformation of competent E. coli BL21 cells with the pGEX-AKAP18delta plasmid, the cells were grown, and protein synthesis was induced with IPTG. Initially, investigations were conducted as to whether and in which way lysis of the bacteria under varying conditions would affect the protein yield.

In addition to variants of the standard method, i.e. lysis with a French press, cells from the same induction culture were lysed with lysozyme and following addition of DTT. The following variants were investigated:
1× French press
3× French press
addition of 5 mM DTT prior to 1× French press lysis
30 min at RT with lysozyme (1 mg/ml)
30 min at 37° C. with lysozyme (1 mg/ml)

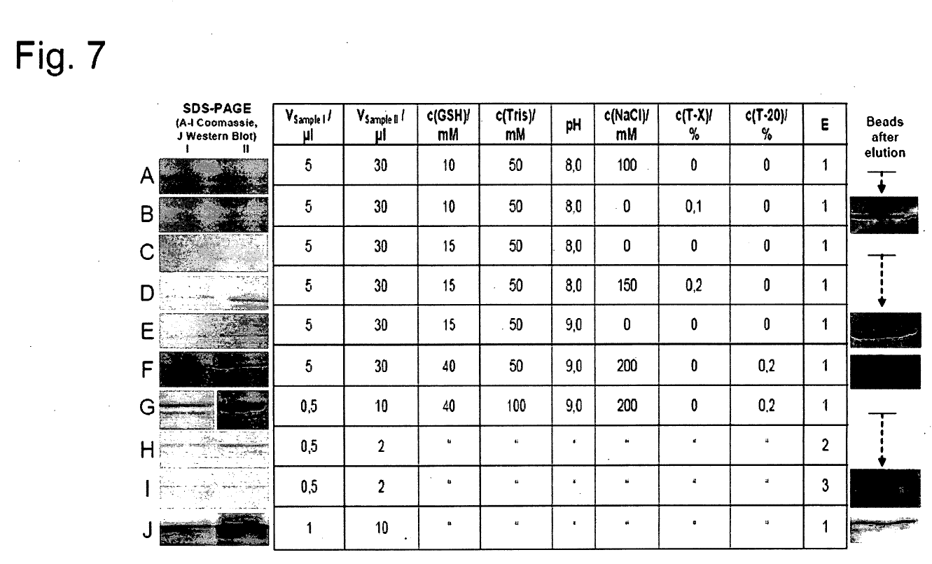

Subsequent to said additional purification procedure and binding to Glutathione Sepharose (see section 1.6), 5 μl of beads were removed from each batch and analyzed using SDS PAGE with subsequent coomassie staining (FIG. 7). Cells transformed with an empty pGEX vector were co-used as control, with GST being the only purification product to be expected for the latter.

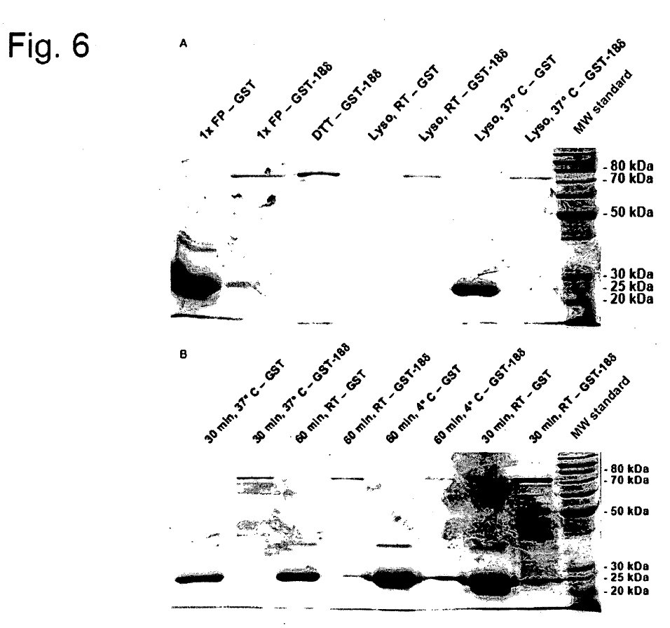

As can be seen from FIGS. 6A and 6B, it was possible to detect proteins of the expected sizes.

Addition of 5 mM DTT to the cell suspension prior to lysis resulted in higher protein yield (FIG. 6A).

Visual comparison of the coomassie-stained gels showed that lysozyme was less effective in cell lysis than the French press (FIG. 6A).

The highest yield of proteins was achieved by incubation at room temperature for 30 min (FIG. 6B).

### 2.1.3 Binding Efficiency of the GST Fusion Protein to Glutathione Sepharose was Improved

An attempt was made to optimize the efficiency of binding GST-AKAP18delta to Glutathione Sepharose by varying the incubation time and incubation temperature of the cell lysate with Sepharose beads (FIG. 6B).

Incubation of the cell lysate at room temperature for 30 min was shown to furnish the highest protein yield.

2.1.4 The Elution Conditions were Optimized

Elution of GST-AKAP18delta from the Sepharose beads was found to be scarcely effective under standard conditions, so that the conditions of elution were adapted so as to maximize the protein yield.

Varying the elution time and temperature in comparison to the manufacturer's instructions (10 min at room temperature) failed to result in improvements, for which reason the composition of the elution buffer was changed. According to the manufacturer's recommendations, the composition of the latter is 10 mM glutathione (GSH) in 50 mM Tris-HCl, pH 8.0. Addition of 100 mM NaCl or 0.1% Triton X-100 was found to give no increase in effectiveness (FIGS. 7, A and B), so that the GSH concentration was increased to 40 mM in steps, the Tris concentration to 100 mM, the pH value to 9.0, and the NaCl concentration to 200 mM. Furthermore, the elution buffer was added with 0.2% Tween 20. Finally, tests were made to see if more protein would be obtained by multiple elutions (FIG. 7, C-I).

Using Western blotting (with A18delta3 and peroxidase (POD)-coupled anti-rabbit Ab), the purified protein was identified as GST-AKAP18delta (FIG. 7J).

The protein concentration of a purification performed prior to optimizing and utilized for establishing an ELISA was determined using a Bradford assay and found to be 0.4 μg/μl.

For screening, another purification of the recombinant GST-AKAP18delta protein was required, which was carried out under conditions optimized as above. The concentration of the eluate from this purification was determined by comparison with the first purification using the established ELISA and found to be 8 μg/μl.

### 2.2 Establishing an ELISA to Quantify Protein-Protein Interactions

For quantitative detection of protein-protein binding between the RIIalpha subunit of PKA and GST-AKAP18delta, an ELISA-based detection was developed which, appropriately optimized by routine tests, can be used for all the other AKAP proteins and PKA subunits.

As already described in section 1.10, the advantages of this method are, in particular, high sensitivity and easy handling. In addition, the substances required had already been established and were available in sufficient quantities. While the anti-rabbit POD Ab and chemiluminescence solution were commercially available, the A18delta3 Ab and GST-AKAP18delta were produced in-house (see above).

First of all, the principal question was if GST-AKAP18delta should first be bound to the microtiter plates (MTP) and binding of RIIalpha detected subsequently or vice versa. The former option implies lower sensitivity and higher protein demand, and the required PKA_{RIIalpha} antibody cannot not be self-produced, for which reason it was only the second option, i.e., binding of RIIalpha to the MTPs and subsequent detection of GST-AKAP18delta binding thereto, that was pursued after the initial steps of establishment.

In developing the assay, the following important aspects required investigation:
Composition of suitable binding or blocking buffer
Amount of RIIalpha protein to be bound to the MTPs
Amount of GST-AKAP18delta binding to plate-bound RIIalpha
Suitable dilution of antibodies for detection
Influence of the dimethyl sulfoxide (DMSO) solvent on ELISA (peptides and organic substances were dissolved in DMSO)
Concentration of inhibitory peptides for control reactions
incubation time of RIIalpha, GST-AKAP18delta, antibodies, and solution for chemiluminescent detection
Conditions of storage for MTPs with bound protein
Detection of chemiluminescence

Establishing an ELISA, observing the above aspects, will be described below. Unless otherwise stated, double determinations were performed in all experiments of establishment, i.e., two wells including the same amount of protein were investigated under the same conditions each time.

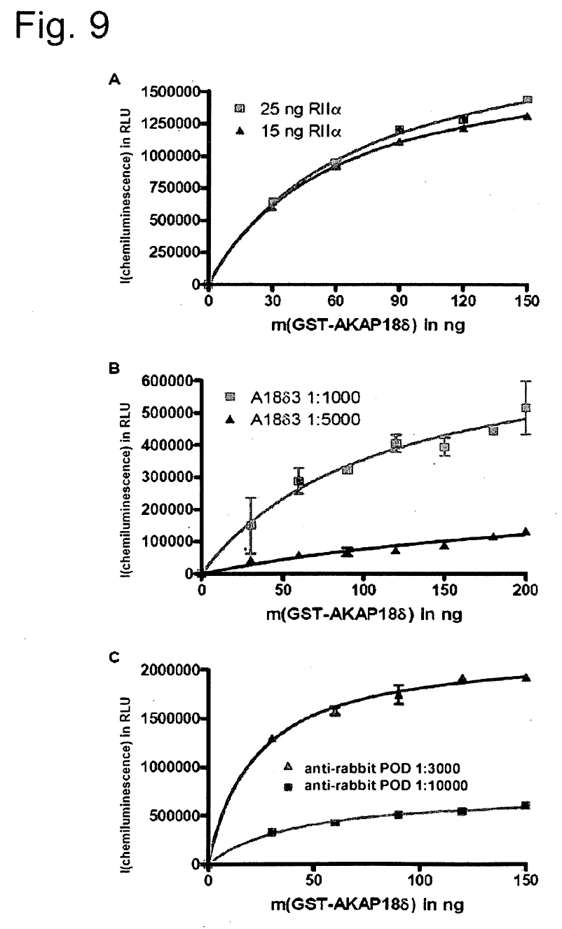

Although it was only two measurements that were performed in each experiment, the standard error was calculated for each mean value to obtain a measure for the precision of the mean value. The data should not be used to determine significances. The error indicators in the drawings represent the standard error of the mean value; in some cases (e.g. FIG. 9A), the standard errors of the mean values were so small that the indicators cannot be recognized.

### 2.2.1 Skim Milk is Best Suited to Block Free Binding Sites on MTPs

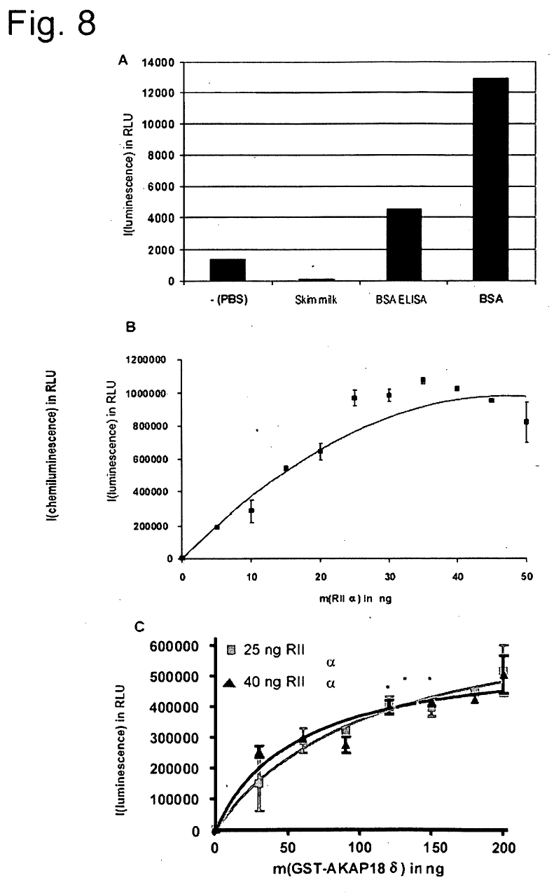

Skim milk powder, bovine serum albumin (BSA), and BSA specially pretreated for ELISA applications (ELISA BSA) were examined as blocking reagents (FIG. 8A; single determinations).

Frequently, BSA includes substances generating non-specific luminescence signals, as demonstrated herein as well. However, even ELISA BSA showed considerable non-specific signals, while skim milk powder virtually gave no chemiluminescence. Therefore, skim milk powder was used at a final concentration of 0.3% in binding buffer to block free binding sites on the MTPs.

2.2.2 An MTP Well can be Saturated with 50 ng of PKA-RIIalpha

Binding buffer solutions with increasing RIIalpha concentrations were pipetted into the wells of an MTP. After blocking free binding sites with skim milk solution, the amounts of protein bound to the plates were detected with the PKA_{RIIalpha} Ab and anti-mouse POD Ab. The optimum antibody concentrations were unknown at that point, so that dilutions of 1:5,000 (PKA_{RIIalpha}) and 1:10,000 (anti-mouse POD) were employed. The measured chemiluminescence intensities were plotted versus the protein concentration to determine if and when saturation of the wells with protein had been achieved (FIG. 8B).

From 45 ng of RIIalpha per well on, the binding capacity was saturated. Next, tests using RIIalpha quantities of 40 ng/well and 25 ng/well were performed.

2.2.3 The Chemiluminescence Intensity Reaches a Maximum at 200 ng GST-AKAP18delta Per Well

25 ng or 40 ng of RIIalpha was bound to the wells of two rows of an MTP each time. Following blocking, blocking buffer solutions including increasing GST-AKAP18delta concentrations (between 0 and 200 ng/well) were pipetted into the wells. This test array will be referred to as binding series hereinafter. Detection of bound protein was effected using A18delta3 and anti-rabbit POD Abs (1:5,000 and 1:10,000, respectively). The signal intensities measured were plotted versus the mass of GST-AKAP18delta per well (FIG. 8C).

From 200 ng GST-AKAP18delta per well on, the binding curve was found to be close to saturation. An initial evaluation of the measured values led to the conclusion that a value of 80 ng GST-AKAP18delta per well would achieve half-maximum saturation. Half-maximum saturation of binding achieves the highest possible sensitivity. Consequently, the following experiments were performed using a GST-AKAP18delta concentration of 80 ng/well.

Further evaluation including the adaptation of the one-site binding model (see section 1.11) to the measured values is shown in FIG. 8C; a lower value of 50-60 ng of GST-AKAP18delta for half-maximum saturation was concluded therefrom.

There were no significant differences between the luminescence intensities of wells coated with 25 ng of RIIalpha and wells coated with 40 ng of RIIalpha, so that the following tests were performed using 25 ng RIIalpha/well to save protein.

To save protein, further investigations were performed to see if an RIIalpha quantity of 15 ng/well would also be sufficient for screening (FIG. 9A). Similarly, no impairment of the luminescence intensity was observed, so that screening was possible with the above amount of protein.

2.2.4 The A18delta3 Antibody was Diluted 1:1,000

As in the test above, an RIIalpha-GST-AKAP18delta binding series was prepared and subsequently detected using varying dilutions of the A18delta3 Ab (FIG. 9B). Initially, the dilution of the anti-rabbit POD Ab was maintained at 1:10,000. At a dilution of 1:5,000, the binding curves showed that not all of the GST-AKAP18delta-RIIalpha complexes were detected. Markedly stronger signals were obtained at a concentration increased by five times. The background signal, i.e., the luminescence intensity at 0 ng of GST-AKAP18delta, remained low at the same time, so that higher sensitivity was obtained.

### 2.2.5 A Reasonable Dilution for the Secondary Antibody is 1:3,000

The same test as in the previous section was performed, in which case the dilution of the A18delta3 primary antibody was 1:1,000 (FIG. 9C). Similarly, an increase in signal intensity and sensitivity was observed for the anti-rabbit POD Ab when decreasing the dilution from 1:10,000 to 1:3,000.

Consequently, the following experiments and the substance screening were performed using the above-determined dilutions of 1:1,000 for the A18delta3 Ab and 1:3,000 for the anti-rabbit POD Ab.

### 2.2.6 No Impairment of the ELISA by DMSO

The influence of the organic solvent dimethyl sulfoxide (DMSO) on protein-protein binding or detection thereof required investigation because the inhibitory peptides L314E and Ht31 used as controls, as well as the substance library itself, were dissolved in DMSO.

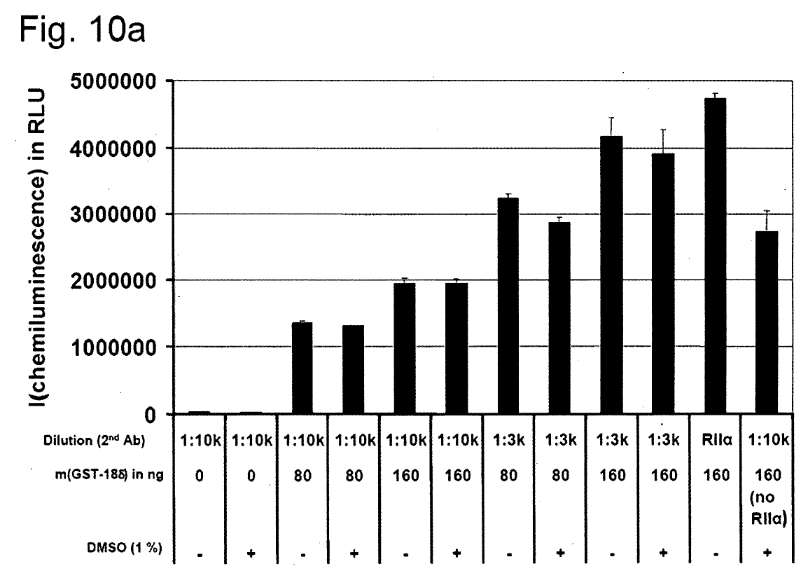

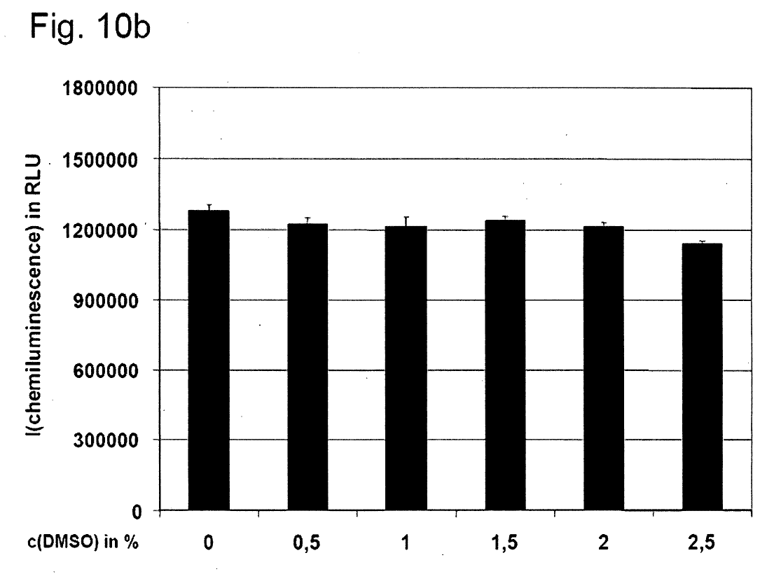

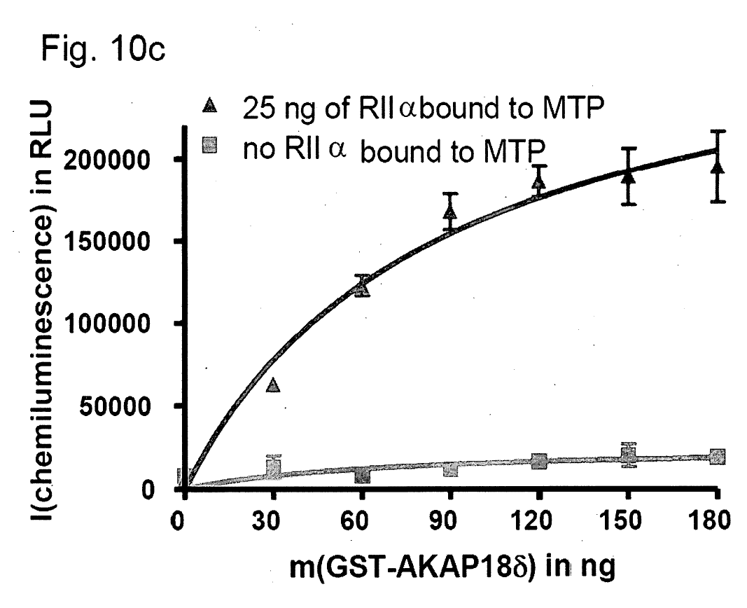

In an initial experiment, the influence of 1% DMSO, relative to the overall reaction volume (20 μl), was tested, likewise using varying dilutions of secondary Ab and concentrations of GST-AKAP18delta at the same time (FIG. 10A). It was found that DMSO had no substantial influence on the results; the results previously obtained for varying GST-AKAP18delta concentrations and Ab dilutions were confirmed.

In another experiment, wherein constant amounts of RIIalpha and GST-AKAP18delta were added with increasing DMSO concentrations of up to 2.5% (FIG. 10B), the signal obtained had only slightly decreased. For screening, the maximum DMSO concentration was also 2.5%.

In a control experiment, the RIIalpha bound to MTP was detected using the PKA_{RIIalpha} and anti-mouse POD Abs (FIG. 10A, penultimate value).

In another control (likewise FIG. 10A, last value), no RIIalpha was bound to the plate and the rest of the assay was performed as already known. Surprisingly, high chemiluminescence intensity was measured in this case, leading to the question whether the signals measured would reflect the amount of bound GST-AKAP18delta at all. For examination, another experiment was performed (see next section).

2.2.7 Detection of RIIalpha-Bound GST-AKAP18delta is Specific

To control the signal specificity, 25 ng RIIalpha/well was bound in one row of an MTP, whereas a second row had no RIIalpha. After blocking non-specific binding sites, solutions including increasing GST-AKAP18delta concentrations were pipetted into the two rows, and specifically bound protein was detected by means of the antibodies (FIG. 10C).

While the signals in the RIIalpha row increased and finally reached saturation, only slight increase due to non-specific binding of GST-AKAP18delta was observed in the row with no RIIalpha. This result showed that detection was specific. Thus, the control in FIG. 10A, last value, probably involves a pipetting or other error.

2.2.8 Inhibitory Peptides Prevent Binding of GST-AKAP18delta to RIIalpha

As is well-known, binding between AKAPs and R subunits of PKA can be inhibited in a specific and competitive fashion by means of AKAP-derived peptides. Consequently, such peptides were used in control reactions with the intention of preventing the interaction of RIIalpha and GST-AKAP18delta with peptides. To obtain detection as sensitive as possible in this case as well, the IC₅₀ values for the established anchor inhibitor peptide Ht31 and for AKAP18-L314E were determined first. The IC₅₀ value is the peptide concentration where only 50% of the maximum possible protein-protein complexes are formed.

To determine these values, RIIalpha was coupled to an MTP at 25 ng/well, and 80 ng of GST-AKAP18delta was added each time. Thereafter, the peptides were added at increasing concentrations by pipetting.

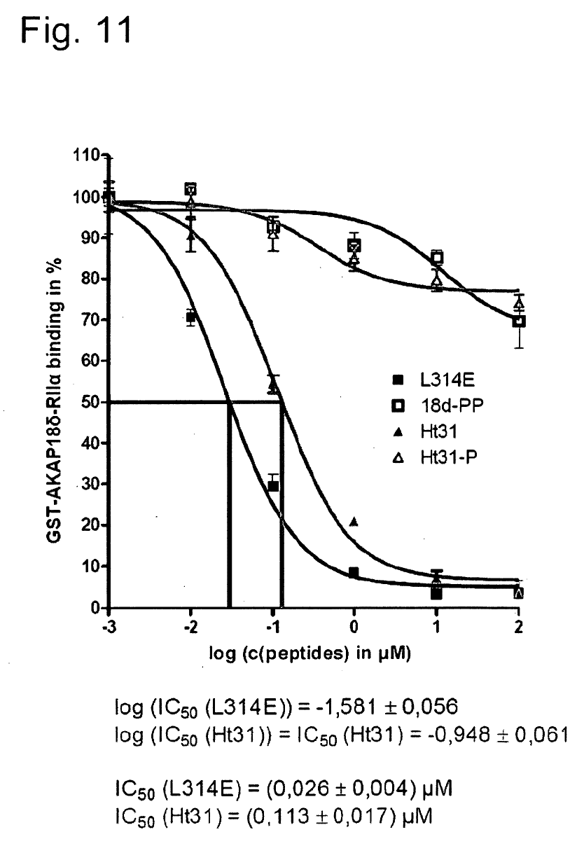

The L314E peptide gave stronger inhibition of binding, for which reason it was employed in the control reactions at a concentration around IC₅₀ of 25 nM (FIG. 11). Peptide inhibition was specific, because the proline-containing control peptides 18d-PP and Ht31-P gave no or only weak inhibition.

### 2.2.9 An Incubation Time of 30-60 min is Sufficient for Protein-Protein Binding

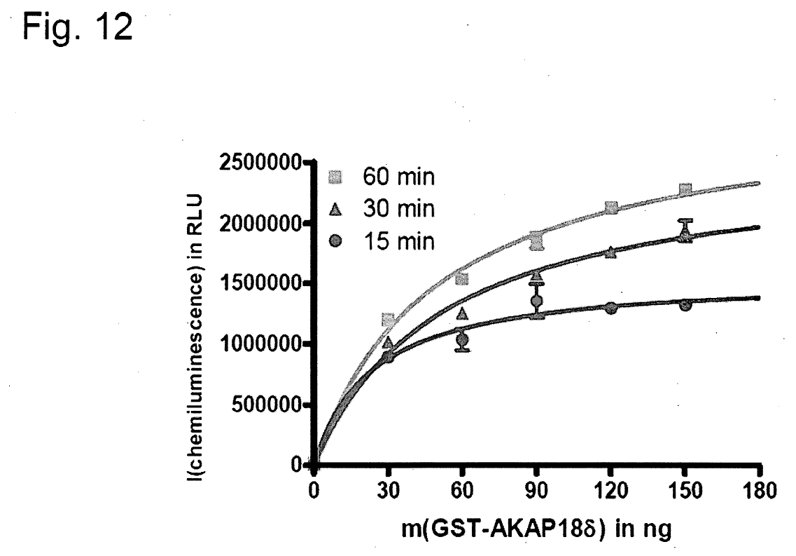

The factor time plays a not inconsiderable role in planning and implementing a substance library screening. For this reason, some additional experiments were performed, providing information as to the required incubation times. First of all, the amount of time to form protein complexes sufficient to obtain adequate chemiluminescence intensity was tested. Again, binding series were pipetted to this effect, and the formation of protein-protein bonds was interrupted after varying amounts of time—15, 30 and 60 min—by washing the MTPs. Thereafter, the amount of bound GST-AKAP18delta was detected using the Abs (FIG. 12).

Strong chemiluminescence signals were detected after only 30-60 min.

### 2.2.10 A Time of 30-60 min is Sufficient for Inhibition by Inhibitory Peptides

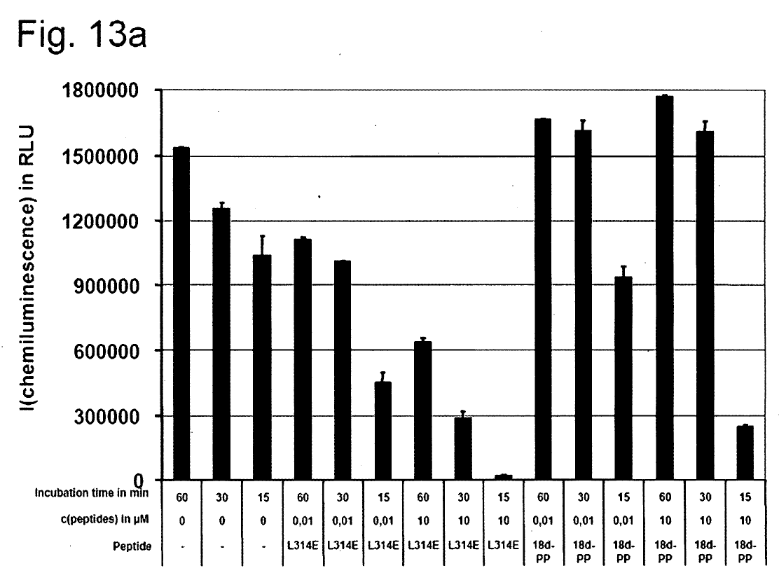

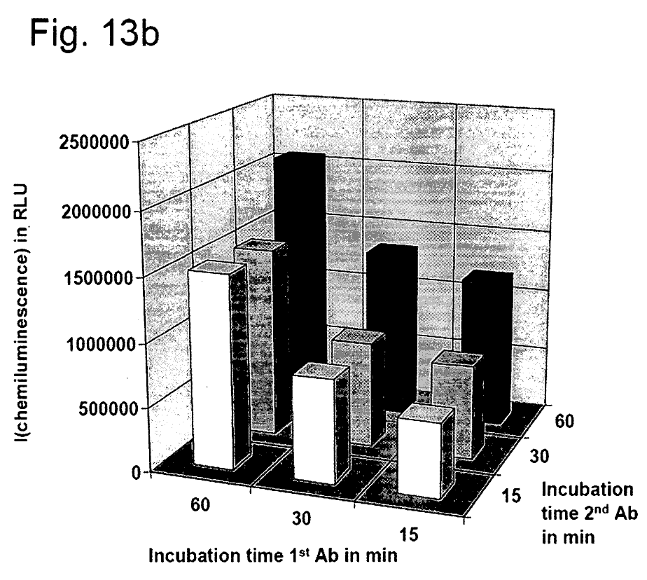

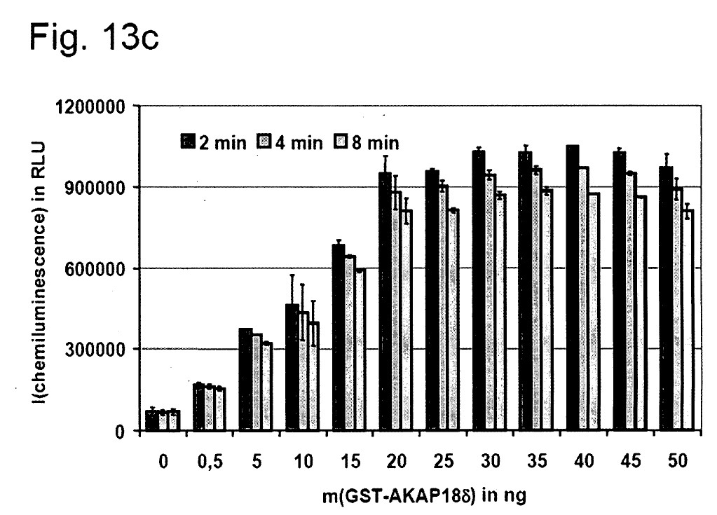

Further, binding of GST-AKAP18delta to RIIalpha was investigated in the presence of L314E peptide with competitive inhibition and 18d-PP control peptide. The peptides at concentrations of 0.01 or 10 μM were placed in the reaction batches for 15, 30 or 60 min, followed by detection of the amount of bound GST-AKAP18delta (FIG. 13A). An incubation time of 30-60 min, as previously determined, was found sufficient in this case as well.

### 2.2.11 The Optimum Antibody Incubation Time is 60 or 15 min

Binding of antibodies to their antigens proceeds in a time-dependent fashion, and the specificity initially increases with increasing incubation time. The specificity may drop back at some later point in time due to degradation of proteins, for example. To obtain the incubation period required for sensitive measurements, RIIalpha-GST-AKAP18delta complexes were added with A18delta3 and anti-rabbit POD antibodies, and the binding reactions were interrupted after 15, 30 and 60 min by washing, taking into account all possible combinations of incubation times for the primary and secondary antibodies (FIG. 13B).

Incubation for 60 min was found to achieve the highest signal intensity with both antibodies. However, reducing the incubation time of the secondary antibody appeared justifiable if the primary antibody was allowed to bind for 60 min.

### 2.2.12 Chemiluminescence can be Detected Over a Period of 8 min

Generation of chemiluminescence by oxidation of luminol is a time-dependent reaction. The intensity drops after some minutes due to consumption of substrate and denaturation of peroxidase in the course of the reaction.

In commercially available products, such as the LumiLight solution used herein, this is not a serious problem when adding auxiliary agents slowing down the drop in intensity. The time window after addition of substrate solution to the reaction batches, during which measurement should be performed, was nevertheless examined. To this end, a binding series was pipetted (see above), and measurement of the chemiluminescence was repeated 2, 4 and 8 min after addition of LumiLight solution (FIG. 13C).

Performing the measurement was possible within 8 min after addition of LumiLight without having to fear dramatic losses in signal intensity. The entire measuring operation was easily completed within the above time window.

### 2.2.13 Protein-Coated MTPs can be Stored for a Prolonged Period of Time

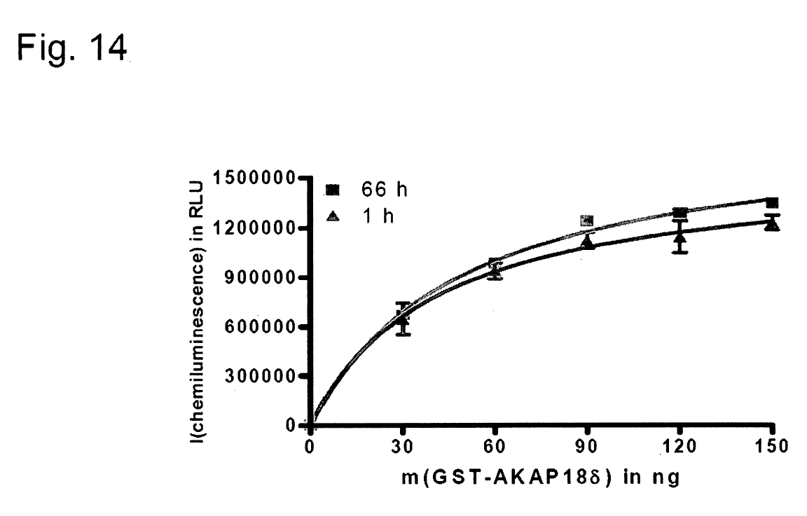

To accelerate screening, it was reasonable to coat several MTPs with RIIalpha and store them for future use. To confirm that the protein would not undergo degradation as a result of storage, RIIalpha was bound to two rows of an MTP as described, and non-specific binding sites were blocked. Thereafter, the ELISA was completed after storage at room temperature for 1 h in one of the rows and at 4° C. for 66 h in another one, and the amount of bound GST-AKAP18delta was detected (FIG. 14).

The storage time had no recognizable influence on the result within the time window under investigation.

Thereafter, screening of the substance library was carried out using the concentrations and incubation times thus determined.

2.3 Screening of a Substance Library to Find Low-Molecular Weight Inhibitors of the RIIalpha-GST-AKAP18delta Interaction

Once the MTPs had been pipetted and measured as described in section 1.12, an initial examination was made to see if the measured chemiluminescence intensities were high enough. If so, the chemiluminescence of each well was converted into the corresponding percent values of the amount of RIIalpha-GST-AKAP18delta binding compared to the control, using the Microsoft Excel table calculation program. To this end, the mean value of the measured values of the controls with 80 ng of GST-AKAP18delta (the wells C1, D1, M24, N24; see Tables 1.1 and 1.2) was set to 100%. To facilitate visual evaluation, the results were subsequently given a color by means of automated color assignment (using a program written in Microsoft Visual Basic), using different colors at 10% intervals. To give an example, Table 2.1 shows the raw data of an MTP (chemiluminescence in RLU) and the associated conversion into percent binding with coloration. The complete data of the screening can be found in the annex.

Table 2.1: Top: Raw data from the measurement of an MTP used in screening, showing the intensity of chemiluminescence in RLU.
Bottom: Using the raw data, the percent binding between GST-AKAP18delta and RIIalpha in the presence of potential inhibitors compared to the controls (the wells C1, D1, M24, N24; see also Table 2.1 for plate occupancy) was calculated and given a color according to the color chart shown below.
$

Once colored, some of the MTPs displayed distinctive features, e.g. many inhibitor candidates in a particular area of the plate. If examination of the control values indicated that the 100% values in columns 1 and 24 varied strongly (more than 20% difference), the corresponding MTP was reevaluated, using for each half of the plate the control values of this half only as basis (e.g. the control values from column 1 for columns 1-12).

If conspicuous areas on the plate nevertheless remained, the entire plate was pipetted, measured and evaluated once more, which was required for the plates 4, 6, 7, 13, 21, 38 and 50.

A so-called Compound ID was assigned to each substance of the library, facilitating computer-based evaluation of the data. Following completion of the screening, all measured values were assigned to corresponding Compound IDs in a table. Sorting the results according to increasing percent binding values furnished the substances illustrated in Table 2.2, the use of which achieved inhibition of protein-protein binding between RIIalpha and GST-AKAP18delta down to 20% or less of the control value.

Table 2.2: Screening of the library comprising 20,064 substances furnished 19 candidates which, at a substance concentration of 244 μM, caused inhibition of binding between RIIalpha and GST-AKAP18delta down to 20% or less compared to the controls. Plate_ID=No. of library MTP, Pos_ID=position of well including candidate substance.
$
Table 2.3: Pipetting scheme and measured values of the validation. A dilution series was made for each potential inhibitor (A7-H15, I1-P10; compound IDs shown) and the peptides (A3-H6, I11-P12) to investigate the concentration dependence of the inhibitory effect. In addition, binding series with no inhibitors were produced (A1-H2, I13-P14). Furthermore, the luminescence intensities were converted into percent values as described in Table 3.1 and labelled with a color (see Table 3.4). Comp. ID=Compound ID, I=intensity.
$

### 2.4 Validation of Potential Inhibitors

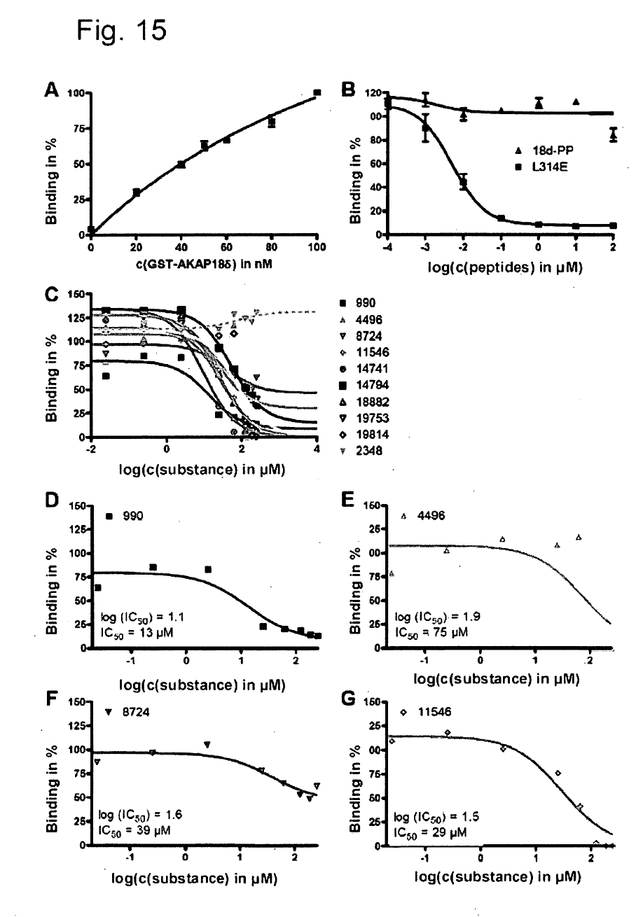

To validate the potential inhibitors found in screening of the substance library, they were selected from the library MTPs, and dilution series together with control reactions were established according to the pipetting scheme shown in Table 2.3. The dilution series were intended to demonstrate the concentration dependence of the potential inhibitors found, which was only to be expected in case of specific inhibition of GST-AKAP18delta-RIIalpha binding. Using the ascertained data, the IC₅₀ values for these compounds (Table 2.4) were calculated as described in section 1.11 (FIG. 15). These values indicate at which concentration of substance half of the maximum possible inhibition is reached. Due to the limited amounts of substances, single determinations were carried out, so that no error indicators are given in FIG. 15, except for the controls.

Identity and purity of the identified substances were confirmed by means of liquid chromatography/mass spectroscopy measurements (LC-MS). The substances were reordered in higher quantities from ChemDiv (San Diego, USA) for further investigations.

Table 2.4: Percent binding between GST-AKAP18delta and RIIalpha in the presence of inhibitory molecules (A7-H15, I1-P10), peptides (A3-H6, I11-P12), or with no inhibitors (binding series in A1-H2, I13-P14), calculated from the results of Table 2.3. The color code corresponds to that in Table 2.1.
$
Table 2.5: Properties of nine substances that showed concentration-dependent inhibition of RIIalpha-GST-AKAP18delta binding in the above validation. MW=molecular weight in g/mol, log P=partition coefficient, H_acceptor=number of atoms which can act as acceptors in hydrogen bridges, H_donor=number of atoms which can act as donors in hydrogen bridges.
$

### 3. Discussion

Protein kinase A anchor proteins (AKAPs) have important functions in cAMP-dependent signaling pathways. They are expressed in a tissue-specific fashion, and various AKAP proteins anchor protein kinase A (PKA) on various subcellular compartments. At the same time, they can be scaffolding proteins for key components of further signaling pathways.

Inter alia, an ELISA for the screening of a library of low-molecular weight, drug-like substances was developed. Using this method, n concentration-dependently effective inhibitors of AKAP18delta-RIIalpha binding were found.

By virtue of the specific inhibition of PKA anchoring, in combination with properties of low-molecular weight substances, said substances could be used not only in the validation of AKAP proteins as potential targets of new active substances, but also represent lead structures for the development thereof.

3.1 The Inhibitors of RIIalpha-AKAP18delta Binding have Pharmacologically Interesting Properties

One important demand on active substances is their capability of passing through biological membranes. Their availability in cells and, in fact, both in organisms and in cell culture models, crucially depends thereon. A precondition for membrane permeability is the lipophilicity of a substance. On the other hand, the driving force of membrane transfer is highest possible concentration on one side of the membrane, which can only be achieved by good solubility in aqueous media, which is inversely proportional to the lipophilicity.

One of several models for predicting the membrane permeability is the distribution of a substance in a 1-octanol-water mixture. The partition coefficient P can be calculated from this distribution:

P=[substance]_{1-octanol}/[substance]_{water}

It is usually the logarithm of this value, log P, that is quoted. Table 2.5 represents the log P values of effective inhibitors. If the value is greater than 1, most of the substance will accumulate in the organic phase, and if it is smaller than 1, most of the substance will accumulate in the aqueous phase. The substances found have log P values greater than 1, so that good membrane permeability is expected for all of them.

One factor that adversely affects the membrane permeability of organic molecules is the capability of forming hydrogen bridges. This type of non-covalent bonds results in a hydrate envelope that surrounds the molecules and must be removed with input of energy prior to passage through the membrane.

However, apart from hydrophobic interactions, hydrogen bridges are the most important forces resulting in non-covalent binding of an active substance to its target structure, i.e. to the RIIalpha subunit of PKA or to AKAP18delta in the present case.

One way of assessing the ability to form hydrogen bridges is a simple count of hydrogen bridge donors and acceptors. The number of hydrogen bridge donors and acceptors is also given in Table 2.5.

Another way is computer-based establishment of a model. Thus, for example, the contribution of hydrogen bridges, among other things, in overall binding was determined for an HIV-1 protease inhibitor by calculating possible directions of movement of the binding partners, taking account of their molecular dynamics. Subsequently, the amount of free energy was analyzed to obtain information as to the forces promoting formation of the protein-inhibitor complex.

Another property that influences the availability of small molecules in cells is the size thereof. The size of a molecule can be approximated by its molecular weight. It was shown that both absorption of a substance and elimination thereof via the bile depend on the molecular weight: lower molecular weights result in improved absorption and reduced elimination via the bile. One essential feature of all substances included in the substance library is their relatively low molecular weight of 250 g/mol on the average (see Table 2.5).

None of the above-mentioned methods alone can provide a reliable prediction, especially because the hydrogen bridge binding potential must be regarded in the context with lipophilicity, for which reason Lipinski et al. have established the so-called Rule of Five. The rule defines which properties of a substance give rise to poor membrane permeability:
more than five H bridge donors (=sum of OH and NH groups)
more than ten H bridge acceptors (sum of N and O atoms)
molecular weight above 500 g/mol
partition coefficient log P>5

The active substances determined by the screening comply with all the conditions above (Table 2.5), i.e., they are membrane-permeable.

Nevertheless, for new active substances and for methodical modification of the structures found, so as to improve their effectiveness, one objective should be lowering these values, if possible; preferred structures according to the invention have such lowered values.

3.2 The Fields of Use of the Inhibitors are Determined by their Specificity

Screening was followed by validation (Table 2.4, FIGS. 2.11 and 2.12), wherein particular inhibitors were shown to inhibit binding of the RIIalpha PKA subunit to GST-AKAP18delta in a concentration-dependent fashion.

All identified substances have hydrophobic aromatic ring systems, so that binding in the hydrophobic pocket formed by the RII subunits is possible.

On the other hand, direct binding to the RII binding domain of the AKAP via hydrogen bridges is also conceivable.

Selected substances bind specifically to AKAP18delta, so that the consequences of suspending the compartmentation of this signaling pathway can also be investigated in vivo (see below). Other substances bind specifically to other AKAP proteins, so that other signaling pathways can be investigated by using these substances.

Advantageous substances bind AKAP18 proteins in a specific fashion, i.e. any of the splicing variants alpha, beta, gamma and delta, thereby excluding them from interaction with RII subunits, because all the isoforms have the same RII binding domain (FIG. 1.5).

However, if one of the substances binds to the RII subunit, a tool is provided by means of which interactions between AKAP proteins and RII subunits can be inhibited in general. The binding sites can be elucidated using computer-based establishment of models wherein the structures of the substances found can be examined with the structures of RIIalpha or of the AKAP-RII binding domain for possible interacting regions.

### 3.3 The AKAP-PKA Inhibitors Represent Lead Structures for new Active Substances

The inhibitors of RIIalpha-GST-AKAP18delta binding are developed into a new class of pharmaceutical agents with advantage.

One way of using inhibitors of PKA-AKAP18delta binding (but also binding to other AKAP molecules) as new pharmaceutical agents results from the involvement of AKAP18delta in AVP-induced translocation of AQP2 into the apical membrane of renal collecting duct cells (AQP2 shuttle).

In this process, AKAP18delta anchors the PKA on AQP2-containing vesicles, so that the catalytic subunits, following AVP stimulation, can phosphorylate the water channel proteins in a specific fashion, resulting in transport to and fusion of the vesicle with the apical plasma membrane. In this way, the water permeability of the membrane is increased.

As demonstrated in a rat model, water retention may occur in particular diseases such as heart failure, liver cirrhosis, hypertension, but also during pregnancy, possibly giving rise to edemas, among other things.

To date, retention of water has been treated using so-called diuretics. Diuretics indirectly provide for increased excretion of water via the kidneys by inhibiting Na⁺, K⁺ and Ca²⁺ ion transporters normally transporting ions from the primary urine back into the collecting duct epithelial cells. As a result, the osmolarity in the collecting duct is increased, and water follows by osmosis through AQP2 molecules constitutively present in the apical cell membrane of the collecting duct epithelial cells. Apart from water retention, diuretics are also used in heart failure and hypertonia. However, undesirable drug effects (UAW) may occur due to massive loss of electrolytes.

One class of pharmaceutical agents void of UAW caused by electrolyte loss are aquaretics. They effect increased water excretion and represent potential substitutes for conventional diuretics. To date, vasopressin receptor antagonists are the only pharmaceutical agents known to have an aquaretic effect.

In view of the involvement of AKAP18delta in AQP2 translocation, inhibitors of RIIalpha-GST-AKAP18delta binding can be developed further to furnish novel aquaretic agents. Decoupling of PKA from the AQP2-bearing vesicles by specific inhibition of AKAP18delta-PKA binding (see section 1.7) interrupts the AVP-mediated signal cascade, so that incorporation of AQP2 molecules in the membrane is inhibited despite the AVP stimulus. In this way, water re-absorption cannot be increased, and more water is excreted via the urine. This mode of action—unlike that of diuretics (see above)—does not impair the ion transport back into the collecting duct epithelial cells. Thus, an active substance having such property would be an aquaretic agent by means of which diuresis could be forced in a specific fashion.

A precondition for the development into a pharmaceutical agent is optimizing the inhibitory molecules. They are effective at lower concentrations, allowing similar use thereof in an animal model in order to examine the expected effect as aquaretic agent. If, for example, rats would excrete more water upon administration of optimized inhibitors, AKAP proteins were validated as potential targets of pharmaceutical agents.

AKAP18delta is not only expressed in the kidneys, but also in other tissues. The expression level is particularly high in the heart where other AKAP proteins assume important functions as well.

As demonstrated in a cell culture model of myocardial cells, AKAP-mediated PKA anchoring is a precondition for β-adrenergic regulation of Ca²⁺ currents via L-type Ca²⁺ channels (Caᵥ1.2 channels).

AKAP18alpha (=AKAP15) binds via a leucine zipper motif to the C terminus of the pore-forming alpha, subunit of the Ca²⁺ channel. Following activation of the β-adrenergic receptor/cAMP signaling pathway, the PKA bound by the AKAP protein releases its catalytic subunits which subsequently can phosphorylate a serine residue in the alpha, subunit of the channel. As a result, the open probability of the channel is increased and the Ca²⁺ influx enhanced. This has a positive inotropic and a positive chronotropic effect.

The same study demonstrated that the use of the anchor inhibitor peptide Ht31 suspends the PKA-dependent increase of the Caᵥ1.2 channel activity activated by β-adrenergic receptors. The low-molecular weight inhibitors according to the invention exhibit the same effect as β-blockers in myocardial cells.

β-Blockers are pharmaceutical agents competitively inhibiting the activity of the neurotransmitters adrenaline and noradrenaline on β-adrenergic receptors of the respective target cells. They are differentiated into beta₁, beta₂ and beta₃ receptors expressed in a tissue-specific fashion. Noradrenaline shows a stronger effect on the first one and a lesser effect on the second one. It is predominantly beta₁ receptors that are expressed in myocardial cells, and there are beta₁-selective (cardioselective) and non-selective β-blockers. β-Blockers have a negative inotropic and negative chronotropic effect on the heart. The result of this is a reduced oxygen demand of the myocardium, so that β-blockers are used in the treatment of coronary heart diseases.

In addition, β-blockers have a relaxing effect on the vascular muscles via an unknown mechanism, which is why they are also used in the treatment of hypertension.

Inhibitors of AKAP binding to RII subunits represent a possible alternative to β-blockers. Blocking the AKAP18alpha-dependent signaling pathway in a specific fashion, they possibly have a more specific effect than β-blockers because the latter are blocking all signaling pathways downstream of the receptor. Furthermore, AKAP18 inhibitors can be used in those cases where β-blockers are contraindicated, e.g. in cases of asthma.

However, the new active substances might give rise to UAW in skeletal muscles because L-type Ca²⁺ channels and AKAP18alpha are also expressed therein.

Apart from myocardial cells, Caᵥ1.2 channels and AKAP18alpha also occur in dendrites and cell bodies of neurons in the brain. Therefore, AKAP18alpha is possibly involved in the regulation of Caᵥ1.2 channels therein as well, opening another potential field of use for the inhibitory molecules.

### 3.4 The Substances Represent New Tools for Blocking PKA Anchoring

Up to now, the protein-protein interaction between AKAP proteins and RII subunits of PKA has been inhibited using peptides (Ht31, AKAP_{IS}), as described in section 1.8. However, specific inhibition of a particular AKAP-RII complex is not possible in this way because the peptides correspond to the RII binding domain of Ht31 or have been generated starting from a consensus binding site calculated from various AKAP proteins, thus blocking all regulatory subunits present in the system under investigation.

All of the RII binding domains of AKAP proteins are very similar, so that the question is whether small molecules would allow specific inhibition of particular AKAP-PKA complexes. On the other hand, the marginal differences of the RII binding domains possibly allow specific inhibition of the AKAP-PKA interaction after optimizing the substances (see below).

Another drawback of said peptides is their lack of membrane permeability. In experiments requiring membrane permeability, e.g. in cell culture models, the peptides therefore had to be acylated.

This disadvantage can be overcome with the aid of the low-molecular weight inhibitors described herein.

The chemical-physical properties of the substances according to the invention allow direct use in cell cultures as well as in animal models, thereby enabling in vivo investigations of the function of AKAP proteins for the first time.

In addition, after identifying starting substances, the virtually unlimited structural diversity of small organic molecules can be utilized in an optimization with the aim of stronger binding to the protein surface, improved stability, less non-specific side effects, and augmented in vivo availability.

The low-molecular weight inhibitors of the preferred RIIalpha-GST-AKAP18delta interaction permit additional experiments providing information as to their properties, their mode of action and their specificity.

First of all, the substances can be examined for non-specific effects on proteins, such as denaturation, using an established ELISA. To this end, RIIalpha or GST-AKAP18delta can be bound to MTP and added with increasing concentrations of substances. Subsequently, the proteins can be detected using the PKA_{RIIalpha} or A18delta3 antibodies.

The ELISA can also be used in an initial examination of the specificity of the low-molecular weight inhibitors by binding RIIalpha to MTP and adding various recombinant AKAP proteins in the presence of increasing inhibitor concentrations. Inhibition of all AKAP-RIIalpha interactions indicates binding of the inhibitor to RIIalpha. Varying levels of inhibition from one AKAP to another AKAP suggest binding of the inhibitors to the marginally different RII binding domains of the AKAP proteins.

Apart from the above investigations, three additional experiments should be mentioned which can be performed immediately, using well-established reagents and methods. They can provide information as to which of the ascertained substances would also be effective in cells, especially when the cells are part of an organism, and therefore could be put to optimization.

Initially, collecting duct cells from the inner medulla of rat kidneys (IMCD cells) can be stimulated with AVP in the presence or absence of the substances so as to stimulate AQP2 translocation. Thereafter, immunofluorescence staining can be performed, wherein the cells—following incubation—are fixed and made permeable. Using antibodies coupled to fluorescent dyes, the intracellular localization of AQP2 can subsequently be determined under a fluorescence microscope. In this way it is possible to investigate the influence of the substances on the presumably AKAP18delta-dependent translocation of AQP2.

Another experiment is measurement of the Ca²⁺ currents through L-type Ca²⁺ channels in primarily cultured myocardial cells from neonatal rats using the patch clamp technique. To this effect, membrane sections of a single cell are fixed by a buffer-filled glass capillary, and a constant voltage is applied across the membrane. A current pulse can trigger a measurable increase of the Ca²⁺ current through the voltage-dependent L-type Ca²⁺ channels situated in the fixed membrane region.

After stimulating the cells with the non-specific β-adrenoreceptor agonist isoproterenol, an increase of the Ca²⁺ channel current is to be expected because receptor activation triggers the AKAP18alpha-dependent signal cascade described in section 3.3. As a result, the L-type Ca²⁺ channel is phosphorylated, thereby increasing its open probability. Such measurements can be performed in the presence or absence of the substances, so that the effect on the signaling pathway can be quantified indirectly via the currents being measured.

Finally, the above-mentioned myocardial cells can be used in another experiment wherein the cells are pre-incubated with isoproterenol and said substances. Thereafter, a membrane-permeable fluorescent Ca²⁺ indicator is added to the cells, which allows visualization of the Ca²⁺ distribution within the cell, and changes of the Ca²⁺ concentration can be analyzed even in a time-resolved fashion, using a laser scanning microscope (LSM). In the presence of the substances, the changes of the Ca²⁺ concentration are expected to be smaller if the substances have an inhibitory effect on the AKAP-PKA interaction and, as a consequence, on the phosphorylation of the Ca²⁺ channel.

If these experiments show that AKAP18delta-specific or non-specific inhibition of the interaction with PKA is possible with the substances in a cell culture model as well, an optimization can be performed with the aim of improving the concentration-dependent effectiveness.

The first step in optimizing is computer-based establishment of a model to obtain a concept as to which functional groups of the molecules would interact with which amino acids in the proteins.

On this basis, functional groups of the molecules can be substituted in a methodical fashion—observing Lipinski's Rule (see section 3.1)—to increase the affinity to the binding partner.

In addition, tests can be made to see if combined use of the inhibitors would achieve an additive or synergistic effect. If improvements can be achieved in this way, targeted synthesis of a molecule can be attempted, which combines functional groups having most effective interaction.

The substances thus obtained would require lower concentrations to achieve the same effect. This allows in vivo use with more advantage, and non-specific or toxic effects are less likely to occur.

The figures illustrate the following matters:

FIG. 1:

Schematic illustration of the pGEX-4T3 vector used to express the GST fusion proteins.

FIG. 2:

The HyperLadder I DNA marker molecular weight standard.

FIG. 3:

The Invitrogen BenchMark protein ladder molecular weight standard for SDS PAGE.

FIG. 4:

Schematic illustration of the ELISA for quantitative detection of protein-protein binding between PKA-RIIalpha and GST-AKAP18delta. In each well of a 384-well MTP (left), one of the following reactions takes place:
A: In the absence of inhibitor, GST-AKAP18delta binds to the plate-bound RIIalpha and can therefore be detected with the antibodies via chemiluminescence.
B: When adding small molecules binding to one of the two binding partners (here: GST-AKAP18delta), both GST-AKAP18delta and antibodies are removed during the subsequent wash steps, so that no chemiluminescence can be generated.
C: The same applies to inhibition of binding by inhibitory peptides which, however, invariably bind to RIIalpha.

FIG. 5:

Competent E. coli cells were transformed with pGEX-AKAP18delta, and the plasmid DNA from 4 clones was prepared, subjected to restriction digestion and analyzed using agarose gel electrophoresis.
Clone 1 non=non-treated plasmid from clone 1, clones 1-4=EcoRI— and XhoI-treated plasmids of clones 1-4.
The digested plasmids show the expected fragments of 4.9 kb and 1 kb.

FIGS. 6, A and B:

Analysis of SDS PAGE sample buffer eluates from Glutathione Sepharose beads having bound thereto recombinant GST-AKAP18delta (75 kDa) or GST (25 kDa) after purification under varying conditions of lysis and binding, using SDS PAGE with coomassie staining. 5 μl of sample buffer eluate was applied each time.
A: Varying the conditions of lysis. GST=product of empty pGEX vector; GST-18delta=product of pGEX-AKAP18delta; FP=French press; DTT=addition of DTT prior to single French press lysis; Lyso=lysis using lysozyme; RT=room temperature; MW standard=molecular weight standard.
B: Varying the conditions of binding. Each bacteria suspension was lysed three times using the French press.

FIG. 7:

By changing the elution buffer composition step by step, it was possible to optimize the elution of GST-AKAP18delta from the Glutathione (GSH) Sepharose beads. Sections from coomassie-stained SDS PAGEs (A-I) and Western blots (J) are shown in the first and last column, respectively. The arrows in the last column indicate the kind of elution conditions used on the beads prior to analysis thereof. The section between 70 and 80 kDa is shown each time. The second and third columns represent the sample volumes applied, the penultimate column represents the eluted fractions and the other columns represent the composition of the elution buffer. V=volume, c=concentration, T-X=Triton X-100, T-20=Tween 20, E=eluate No.

FIG. 8:

A: BSA, BSA-ELISA and skim milk powder were placed in binding buffer. Following addition of chemiluminescent substrate, the intensity of non-specific signals was determined (single determinations).
B: Binding buffer with increasing RIIalpha concentrations (represented as total mass m(RIIalpha)) was pipetted into MTP, non-specific binding sites were blocked with blocking buffer, and the amount of bound protein was detected using PKA_{RIIalpha} and anti-mouse POD Ab.
C: Binding buffer with constant amounts of RIIalpha (25 and 40 ng/well, respectively) was pipetted on MTP, non-specific binding sites were blocked with blocking buffer, blocking buffer including increasing concentrations of GST-AKAP18delta was added, and the amount of bound GST-AKAP18delta was detected using A18delta3 and anti-rabbit POD Abs. I=intensity, RLU=relative light units, m=mass.

FIG. 9:
A-C: Binding series were prepared (as in FIG. 8C).
   A: The chemiluminescence intensities were determined for binding series including constant RIIalpha amounts of 15 and 25 ng, respectively.
   B: Binding series were detected using varying A18delta3 and constant anti-rabbit POD Ab dilutions.
   C: Binding series were detected using constant A18delta3 and varying anti-rabbit POD Ab dilutions.

FIG. 10:

A: Constant amounts of RIIalpha were added with 0, 80 or 160 ng of GST-AKAP18delta. Detection was effected using constant A18delta3 dilutions and 1:3000 (3 k) or 1:10000 (10 k) dilutions of secondary (2^{nd}) anti-rabbit POD Ab. Each experiment was carried out in the presence (+) or absence (−) of 1% DMSO.
B: Binding of constant amounts of GST-AKAP18delta to constant amounts of RIIalpha was detected in the presence of increasing concentrations of DMSO (from 0 to 2.5%).
C: Binding series including 0 and 25 ng of RIIalpha, respectively, were produced and detected in order to identify the specificity of the signals.

FIG. 11:

To calculate the IC₅₀ values, binding between constant amounts of RIIalpha and GST-AKAP18delta was detected in the presence of increasing peptide concentrations. A one-site binding model was adapted to the measured values (see section 1.11). While L314E and Ht31 gave competitive inhibition of binding, 18d-PP and Ht31-P were used as negative controls.

FIG. 12:

Binding series were produced wherein development of RIIalpha-GST-AKAP18delta interaction was interrupted after 15, 30 or 60 min by washing. Thereafter, the amount of bound GST-AKAP18delta was detected.

FIG. 13:

A: Constant amounts of RIIalpha and GST-AKAP18delta were contacted with the peptides L314E and 18d-PP at a concentration of 0.01 or 10 μM for 15, 30 or 60 min. Thereafter, the amount of bound GST-AKAP18delta was detected.
B: RIIalpha-GST-AKAP18delta complexes at constant concentrations were detected with A18delta3 and anti-rabbit POD Abs using all possible combinations of 15, 30 and 60 min of incubation time. Illustration of error indicators was not possible in this Figure.
C: The same RIIalpha-GST-AKAP18delta binding series was added with the Abs and chemiluminescent substrate. The chemiluminescence intensity was measured after 2, 4 and 8 min.

FIG. 14:

MTPs were coated with equal amounts of RIIalpha for 1 h. Blocking buffer was subsequently added, and the MTPs were incubated for 1 h at room temperature or for 66 h at 4° C. Thereafter, GST-AKAP18delta was added at increasing concentrations. The amount of bound GST-AKAP18delta was detected using A18delta3 and anti-rabbit POD Abs.

FIG. 15:

A: The RIIalpha-GST-AKAP18delta binding curve shows that the other experiments, each of which performed using 50 nM GST-AKAP18delta, were in the sensitive measuring range.
B: The use of the inhibitory peptide L314E also allowed inhibition of binding in a concentration-dependent fashion.
C: Summary of the validation experiments using increasing concentrations of inhibitor, showing the 9 hits and the non-active compound 2348 for comparison.
D-L: Concentration-dependent effect of each single inhibitor, structural formula and IC₅₀ values (D-L: single determinations) calculated from an adaptation of a one-site binding model (see section 1.11) to the measured values (single determinations, hence no error indicators). Continued (H-L) on next page.

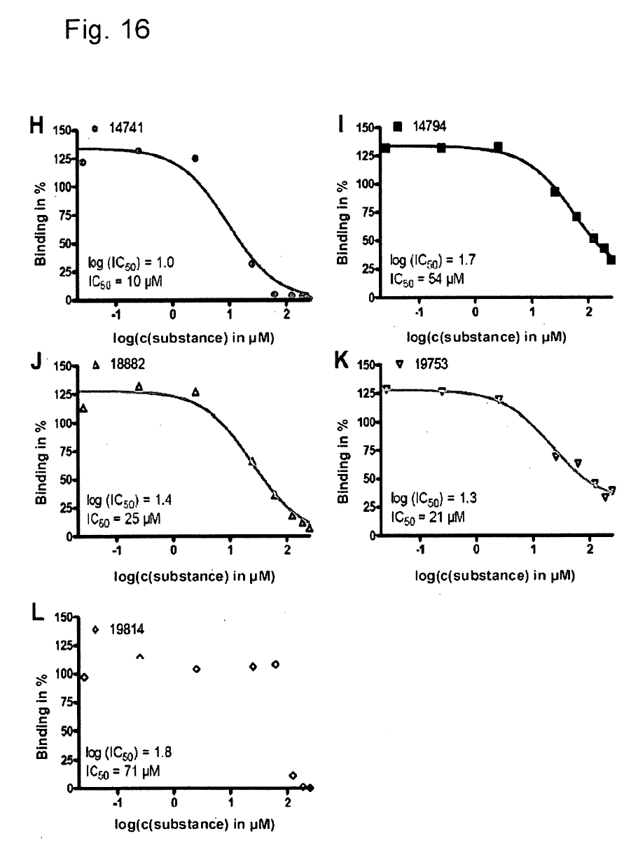

FIG. 16:

Part 2 of FIG. 15. Part 1 and legends see FIG. 15.

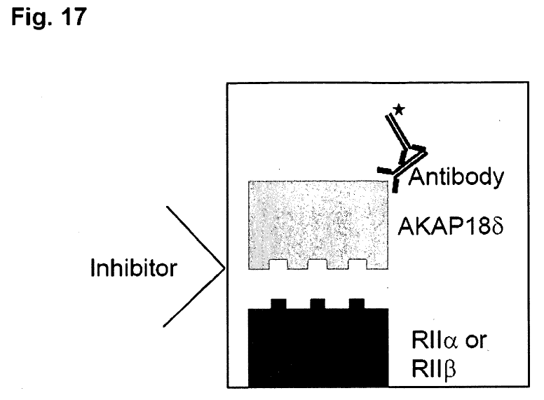

FIG. 17:

Schematic illustration of the ELISA used in quantitative detection of protein-protein binding between PKA-RIIalpha or PKA-RIIbeta and GST-AKAP18delta (glutathione S transferase fusion with AKAP18delta). The interaction proceeds in the absence or presence of low-molecular weight substances in 384-well ELISA plates. Detection of the protein interaction is carried out using primary anti-AKAP18delta and secondary peroxidase-coupled antibodies and chemiluminescence.

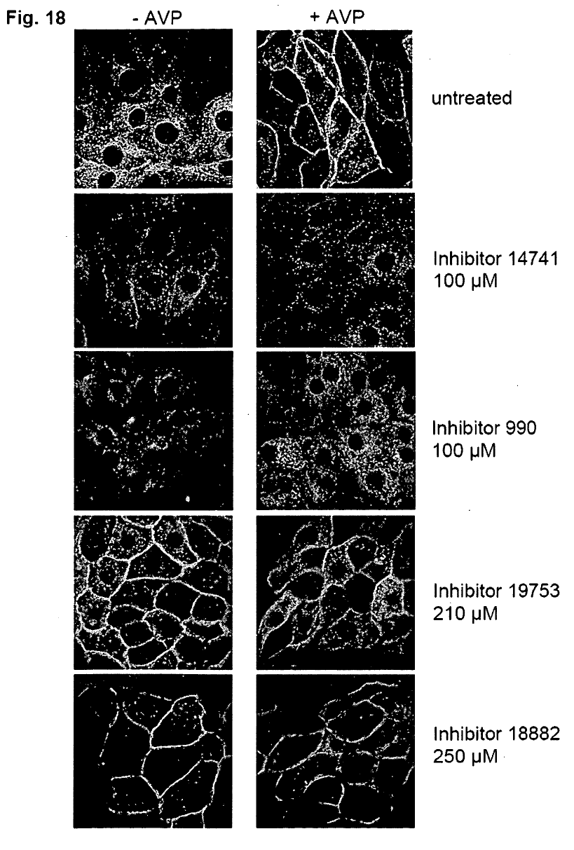

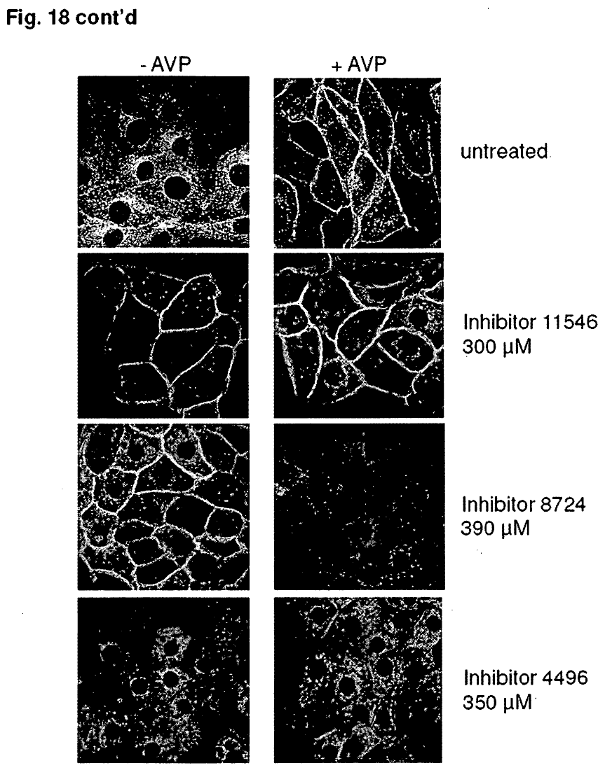

FIG. 18:

Inhibition of the vasopressin (AVP)-dependent redistribution of the aquaporin-2 (AQP2) water channel in renal principal cells by the substances specified (see Table B; concentrations of inhibitors in μM). Primarily cultured principal cells from the inner medulla of rat kidneys (IMCD cells) represent a model for the AVP-induced translocation of AQP2. Said redistribution is the molecular basis of AVP-induced water reabsorption in the kidneys. AQP2 was detected by means of a specific antiserum and Cy3-labelled secondary antibodies in untreated cells (control) and in cells treated with AVP or substances, using a laser scanning microscope (Tamma et al., 2003; Henn et al., 2004).

FIG. 19:

Group of substances which inhibit the interaction of AKAP18delta with regulatory RIIalpha subunits of PKA. All substances inhibiting the interaction (see Tables A and B) were investigated for structural similarities. The numbers under the designations “structures 1-18” correspond to the Comp_IDs (see Tables A and B). Structural similarities were found between substances 1-4, 5 and 6, 7-9, 11 and 12, 13 and 14, 15 and 16, and between 17 and 18. Substance 10 had no similarity with the other substances inhibiting interaction. Generalizations of the structures are illustrated in FIG. 20.

FIG. 20:

Generalization of the structures 1-17 illustrated in FIG. 19.

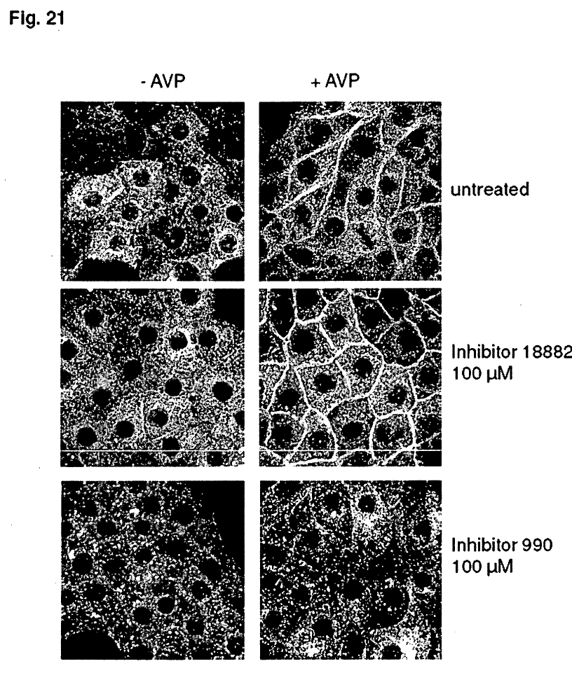

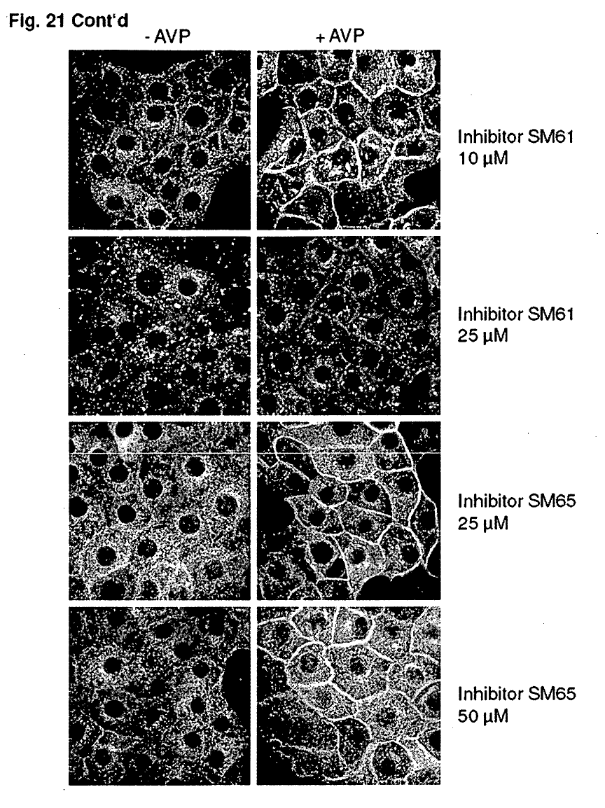

FIG. 21:

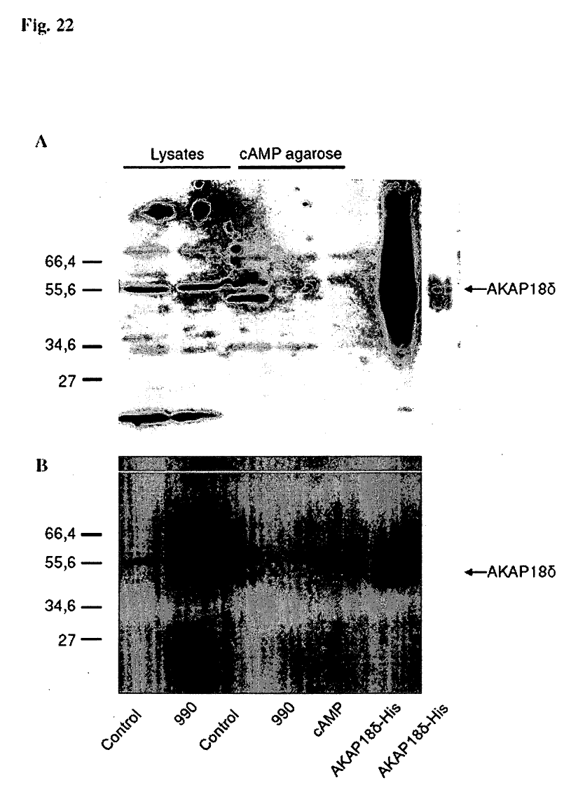

Effect of the low-molecular weight substances 18882, 990 and 990 derivatives SM61 and SM65 on AVP-induced redistribution of AQP2 in IMCD cells (see FIG. 18). The structures and chemical properties of the substances are illustrated in Tables A, B and C (concentrations of inhibitors in μM). AQP2 was detected by means of a specific antiserum and Cy3-labelled secondary antibodies in untreated cells (control) and in cells treated with AVP or substances, using a laser scanning microscope (Tamma et al., 2003; Henn et al., 2004). The low-molecular weight substance 990 inhibits the interaction of AKAP18delta with regulatory RIIalpha subunits of PKA in IMCD cells (see FIG. 22). Correspondingly, 990 prevents the AVP-induced redistribution of AQP2. The substance SM61, a 990 derivative, likewise inhibits the AVP-induced redistribution of AQP2. In contrast, the substances 18882 and the 990 derivative SM65 have no influence on AVP-induced AQP2 redistribution at the concentrations used.

FIG. 22:

The low-molecular weight substance 990 inhibits the interaction of AKAP18delta with regulatory RIIalpha subunits of PKA in IMCD cells. The substance (see Tables A, B and D) was identified by screening a substance library comprising 20,064 compounds, using the ELISA-based test array illustrated in FIG. 17. Primarily cultured IMCD cells either remained untreated (control) or were incubated for 30 min with substance 990 (100 μM) or with cAMP as negative control. Subsequently, the cells were lysed and subjected to cAMP agarose precipitation (Henn et al., JBC 279, 26654, 2004).

A: AKAP18delta was detected in the lysates and in the cAMP agarose precipitates (cAMP agarose) using the AKAP18delta-specific antibody A18delta4 in a Western blot (Henn et al., 2004). cAMP was added to the batch as negative control. As a result, binding of regulatory subunits of PKA to the cAMP agarose was inhibited in a competitive fashion. For this reason, no AKAPs are precipitated and, as a consequence, AKAP18delta cannot be detected in this sample. AKAP18delta produced in a recombinant fashion was used as positive control.

B: To detect the AKAPs in the lysates and cAMP agarose precipitates, an RII overlay was carried out (Henn et al., JBC 279, 26654, 2004). AKAP18delta produced in a recombinant fashion was detected as positive control. AKAP18delta is indicated by arrows in A and B.

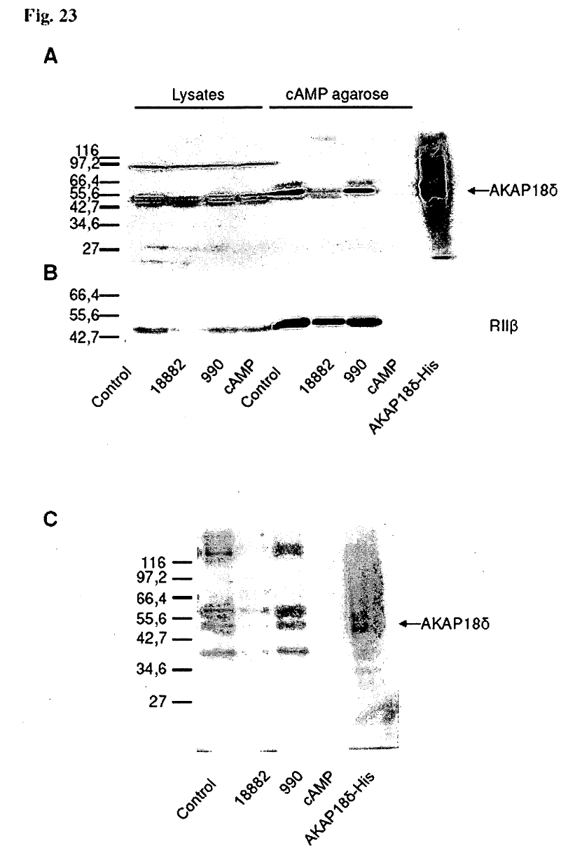

FIG. 23:

The low-molecular weight substance 18882 inhibits the interaction of AKAP18delta with regulatory RIIalpha subunits of PKA in myocardial cells. In contrast, substance 990 has no influence on this interaction. The substances (see Tables A, B and D) were identified by screening a substance library comprising 20,064 compounds, using the ELISA-based test array illustrated in FIG. 17. Primarily cultured neonatal myocardial cells either remained untreated (control) or were incubated for 30 min with the substances 990, 18882 (100 μM each time) or with cAMP as negative control. Subsequently, the cells were lysed and subjected to cAMP agarose precipitation (Henn et al., JBC 279, 26654, 2004).
A: AKAP18delta was detected in the lysates and in the cAMP agarose precipitates (cAMP agarose), using the AKAP18delta-specific antibody A18delta4 in a Western blot (Henn et al., JBC 279, 26654, 2004). cAMP was added to the batch as negative control. As a result, binding of regulatory subunits of PKA to the cAMP agarose was inhibited in a competitive fashion. For this reason, no AKAPs are precipitated and, as a consequence, AKAP18delta cannot be detected in this sample. AKAP18delta produced in a recombinant fashion was used as positive control.
B: Using Western blotting, regulatory RIIbeta subunits of PKA were detected in the same samples as specified in A. As expected, no RIIbeta is detected in the sample containing cAMP (see A.). As expected, the antibody does not recognize AKAP18delta.
C: For non-selective detection of AKAPs in the lysates and cAMP agarose precipitates, an RII overlay was carried out (Henn et al., JBC 279, 26654, 2004). AKAP18delta produced in a recombinant fashion was detected as positive control.

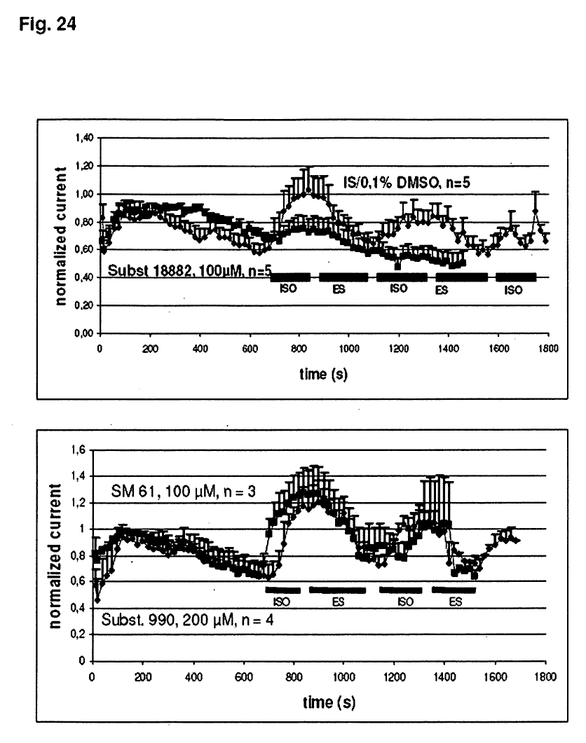

FIG. 24:

Inhibition of L-type Ca²⁺ channel currents by substance 18882. L-type Ca²⁺ channel currents were measured in neonatal rat myocardial cells using the patch-clamp technique. The cells were held at −70 mV and repeatedly depolarized to a test potential of 0 mV (after an increase to −35 mV for 400 ms). Upper diagram: substance 18882 (see Tables A and B) and the solvent DMSO as a control were added to the cells at the concentrations specified. Lower diagram: The substances 990 and 990 derivative SM61 (see Table C) were added to the cells at the concentrations specified. Currents were measured 400 s after the whole cell configuration had established. The cells were stimulated with isoproterenol (1 μM, ISO; a β-receptor agonist) at the times indicated. Isoproterenol was washed out by the ES medium. The diagram shows time profiles of normalized currents (n corresponds to the number of cells measured, the error bars represent means±SEM).

Tables A, B, C and D illustrate the following matters:

### Table A

Low-molecular weight substances which inhibit the interaction of AKAP18delta with regulatory RIIalpha subunits of PKA. The Table shows 142 substances inhibiting the interaction by at least 40%.

Binding (in %): this value indicates the percentage of relative binding of GST-AKAP18delta to PKA-RIIalpha in the presence of each substance, based on a control (binding of GST-AKAP18delta to PKA-RIIalpha in the absence of substance). The substances were identified by screening a substance library comprising 20,064 compounds, using the ELISA-based test array illustrated in FIG. 17. The structures of the substances are shown. The substances are arranged according to their inhibitory effect. The list starts with substances having the most potent inhibitory effect on the interaction of AKAP18delta with regulatory RIIa subunits of PKA.

MW (g/mol): this value indicates the molecular mass of each substance. The molecular mass also represents an approximation of the size of each substance, which is important with respect to bioavailability (small molecules can pass through cellular membranes more easily).

Comp_ID: these numerals represent the serial code of the Leibniz-Institut für Molekulare Pharmakologie (20,064 in total).

Plate_ID: number of each substance library plate wherein the substance has been stored.

Pos_ID: position on the substance library plate wherein the substance has been stored.

Formula: the empirical formulas specified herein indicate the elemental composition of each substance.

log P: this value is the calculated logarithm of the partition coefficient of each substance. It allows an estimation as to the solubility of each substance in polar (aqueous) and nonpolar (membranous (lipid)) phases, thus providing an initial information as to the bioavailability in cellular systems.

log Sw: this value is the calculated logarithm of the solubility coefficient, giving information as to the solubility of each substance in water.

H_acceptor: indicates the number of hydrogen bridge acceptors (N and O atoms) in each substance.

H_donor: indicates the number of hydrogen bridge donors (NH and OH groups) in each substance. Hydrogen bridges are the most important type of non-covalent binding between small molecules and proteins, for which reason hydrogen bridge donors and acceptors enhance the potential of the substances to interact with proteins.

B_rotN: this value corresponds to the number of atomic bonds in each substance, about which free rotation is possible. A higher number of such bonds increases the conformational flexibility of the substance and thus the probability of proper binding to a protein surface.

In summary, the above data allow predictions as to the bioavailability of chemical substances, as demonstrated by Lipinski et al. (Lipinski, C. A., Lombardo, F., Dominy, B. W., Feeney, P. J., Experimental and computational approaches to estimate solubility and permeability in drug discovery and development settings. Adv. Drug Deliv. Rev. 46, 3-26 (2001)). In addition, the substances should comply with the so-called Rule of Five:
five H bridge donors (sum of OH and NH groups) at maximum;
ten H bridge acceptors (sum of N and O atoms) at maximum;
max. molecular weight: 500 g/mol;
partition coefficient log P≦5.

### Table B

Selected low-molecular weight substances which inhibit the interaction of AKAP18delta with regulatory RIIalpha subunits of PKA. The Table shows 9 substances from Table A, which inhibit the interaction by at least 80%.

Binding (in %): this value indicates the percentage of relative binding of GST-AKAP18delta to PKA-RIIalpha in the presence of each substance, based on a control (binding of GST-AKAP18delta to PKA-RIIalpha in the absence of substance).

The IC₅₀ value was determined by titrating each substance. The IC₅₀ for the inhibition of interaction was determined using the ELISA experiment described in FIG. 17.

MW (g/mol): this value indicates the molecular mass of each substance. The molecular mass also represents an approximation of the size of each substance, which is important with respect to bioavailability (small molecules can pass through cellular membranes more easily).

Comp_ID: these numerals represent the serial code of the Leibniz-Institut für Molekulare Pharmakologie (20,064 in total).

Plate_ID: number of each substance library plate wherein the substance has been stored.

Pos_ID: position on the substance library plate wherein the substance has been stored.

Formula: the empirical formulas specified herein indicate the elemental composition of each substance.

log P: this value is the calculated logarithm of the partition coefficient of each substance. It allows an estimation as to the solubility of each substance in polar (aqueous) and nonpolar (membranous (lipid)) phases, thus providing an initial information as to the bioavailability in cellular systems.

log Sw: this value is the calculated logarithm of the solubility coefficient, giving information as to the solubility of each substance in water.

H_acceptor: indicates the number of hydrogen bridge acceptors (N and O atoms) in each substance.

H_donor: indicates the number of hydrogen bridge donors (NH and OH groups) in each substance. Hydrogen bridges are the most important type of non-covalent binding between small molecules and proteins, for which reason hydrogen bridge donors and acceptors enhance the potential of the substances to interact with proteins.

B_rotN: this value corresponds to the number of atomic bonds in each substance, about which free rotation is possible. A higher number of such bonds increases the conformational flexibility of the substance and thus the probability of proper binding to a protein surface.

### Table C

Low-molecular weight substances which modulate the interaction of AKAP18delta with regulatory RIIalpha subunits of PKA. The substances represent derivatives of substance 990 illustrated in Tables A and B and have been developed from substance 990 by chemical modification. The structures, their empirical formulas, their molecular weights (MW) as well as the log P value, the number of H acceptors and H donors of each substance are shown (explanations as to these parameters see legends of Tables A and B). The inhibitory effect of each substance on the interaction of AKAP18delta with regulatory RIIalpha subunits of PKA was examined up to three times (screenings No. 1-3) in the ELISA experiment illustrated in FIG. 17, and the IC₅₀ (mean in μM) was determined therefrom. The ratio of the IC₅₀ of the derivative and substance 990 was calculated (Ratio IC₅₀ (Comp./990)). Values of >1 reflect more inhibition and values of <1 less inhibition of interaction compared to the original substance. Mean IC₅₀ indicates the average IC₅₀ calculated from the experiments performed and normalized with respect to the mean IC₅₀ of 990. Substances showing no inhibition were given a “−”.

### Table D

Low-molecular weight substances SM-FP and 990 which inhibit the interaction of AKAP18delta with regulatory RIIalpha sub-units of PKA. The substance SM-FP represents a derivative of substance 990 illustrated in Tables A and B. SM-FP is characterized by the presence of a fluorescein group. The structures, their empirical formulas, their molecular weights (MW) as well as the log P value, the number of H acceptors and H donors of each substance are shown (explanations as to these parameters see legends of Tables A and B). The inhibitory effect of each substance on the interaction of AKAP18delta with regulatory RIIalpha subunits of PKA was examined up to three times (screenings No. 1-3) in the ELISA experiment illustrated in FIG. 17, and the IC₅₀ (mean in μM) was determined therefrom. The ratio of the IC₅₀ of the derivative and substance 990 was calculated (Ratio IC₅₀ (Comp./990)). Values of >1 reflect more inhibition and values of <1 less inhibition of interaction compared to the original substance. Mean IC₅₀ indicates the average IC₅₀ calculated from the experiments performed and normalized with respect to the mean IC₅₀ of 990. Substances showing no inhibition were given a “−”.

TABLE A
Low-molecular weight substances used to inhibit the AKAP18δ-RIIα interaction

| | MW | | | | |
|---|---|---|---|---|---|
| Structures | g/mol | Comp_ID | PlateID_384 | PosID_384 | Binding |
| 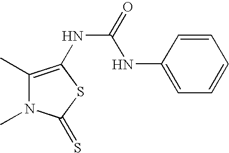 | 279.388 | 19814 | 57 | F08 | 0.27 |
| 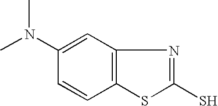 | 210.325 | 4496 | 13 | P18 | 0.64 |
| 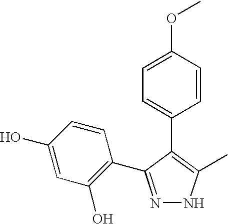 | 296.326 | 8724 | 25 | D19 | 1.27 |
| 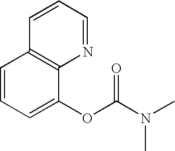 | 216.24 | 95 | 1 | O07 | 1.75 |
| 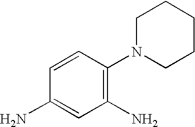 | 191.278 | 14741 | 42 | E21 | 2.30 |
| 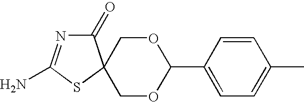 | 278.332 | 19015 | 55 | G02 | 2.49 |
| 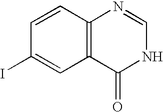 | 272.045 | 256 | 1 | P17 | 2.53 |
| 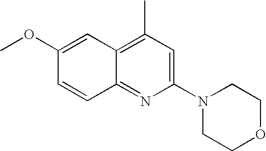 | 258.321 | 288 | 1 | P19 | 3.05 |
| 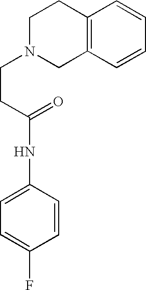 | 298.361 | 10868 | 31 | D21 | 3.46 |
| 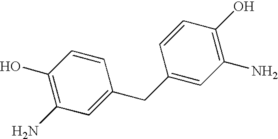 | 230.267 | 18882 | 54 | B16 | 3.53 |
| 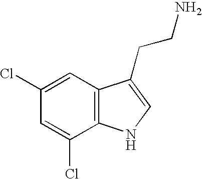 | 229.11 | 272 | 1 | P18 | 5.24 |
| 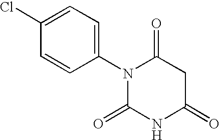 | 238.63 | 12840 | 37 | H12 | 6.77 |
| 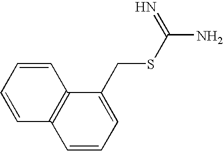 | 216.308 | 990 | 3 | N19 | 12.43 |
| 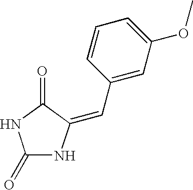 | 218.212 | 11120 | 32 | P14 | 12.61 |
| 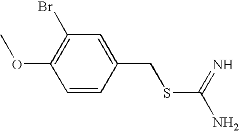 | 275.17 | 2348 | 7 | L16 | 12.98 |
| 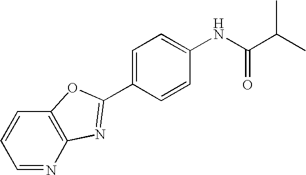 | 281.315 | 16247 | 47 | G05 | 14.34 |
| 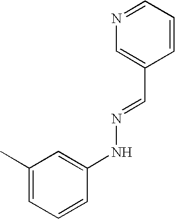 | 211.268 | 11546 | 33 | J19 | 16.36 |
| 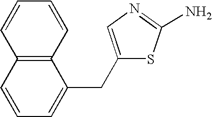 | 240.33 | 14794 | 43 | J02 | 17.71 |
| 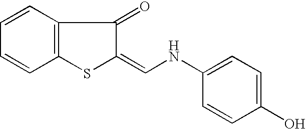 | 269.324 | 19753 | 57 | I04 | 19.35 |
| 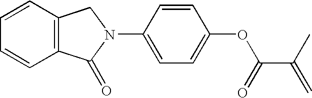 | 293.322 | 16212 | 47 | D03 | 20.67 |
| 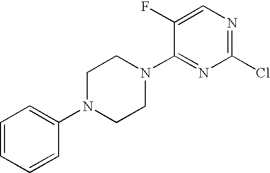 | 292.745 | 4186 | 12 | J21 | 21.85 |
| 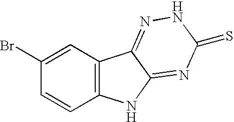 | 281.137 | 3502 | 10 | N22 | 22.33 |
| 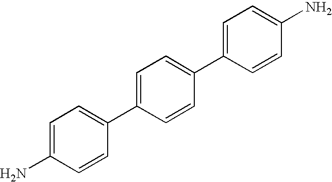 | 260.34 | 19588 | 56 | D16 | 24.43 |
| 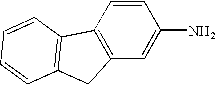 | 181.238 | 18803 | 54 | C11 | 24.88 |
| 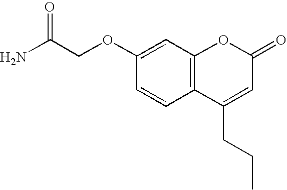 | 261.277 | 16184 | 46 | H23 | 26.39 |
| 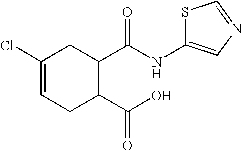 | 286.739 | 1749 | 5 | E23 | 30.81 |
| 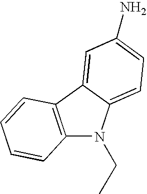 | 210.28 | 19919 | 57 | O14 | 32.36 |
| 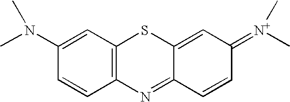 | 284.407 | 18310 | 53 | F02 | 32.50 |
| 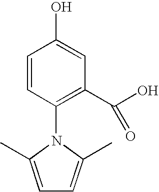 | 231.251 | 15643 | 45 | K11 | 33.97 |
| 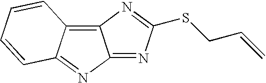 | 227.291 | 3313 | 10 | A11 | 34.31 |
| 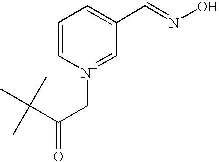 | 221.28 | 240 | 1 | P16 | 34.33 |
| 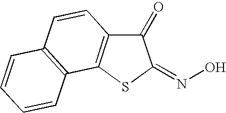 | 229.259 | 3878 | 12 | F02 | 34.49 |
| 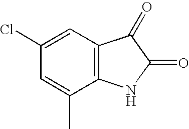 | 195.605 | 17607 | 51 | G02 | 36.10 |
| 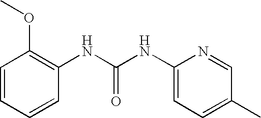 | 257.293 | 17554 | 50 | B21 | 36.81 |
| 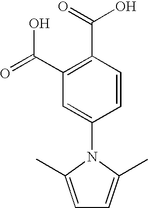 | 259.261 | 19002 | 54 | J23 | 37.08 |
| 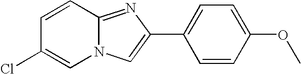 | 258.708 | 15140 | 44 | D02 | 37.54 |
| 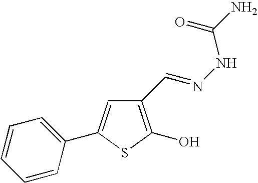 | 261.305 | 14647 | 42 | G15 | 38.05 |
| 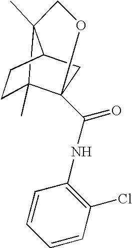 | 305.805 | 16216 | 47 | H03 | 39.29 |
| 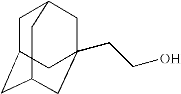 | 180.291 | 224 | 1 | P15 | 39.75 |
| 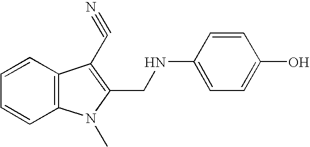 | 277.327 | 9490 | 27 | B23 | 40.72 |
| 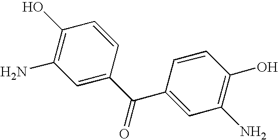 | 244.25 | 14160 | 41 | P06 | 40.82 |
| 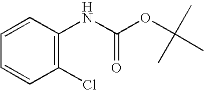 | 227.691 | 12641 | 36 | A22 | 42.26 |
| 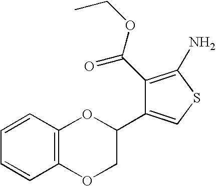 | 305.354 | 16248 | 47 | H05 | 42.52 |
| 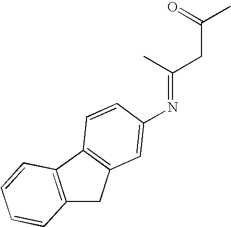 | 263.34 | 17958 | 52 | F02 | 42.56 |
| 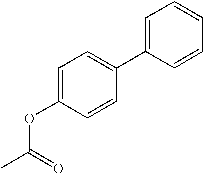 | 212.248 | 18707 | 54 | C05 | 42.71 |
| 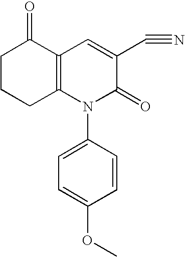 | 294.31 | 9007 | 26 | O14 | 43.06 |
| 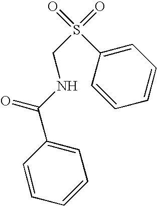 | 275.328 | 5948 | 17 | L21 | 43.10 |
| 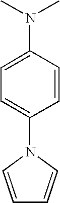 | 186.258 | 4101 | 12 | E16 | 43.48 |
| 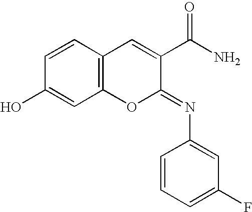 | 298.273 | 14617 | 42 | I13 | 43.50 |
| 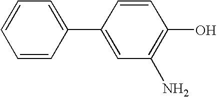 | 185.226 | 14542 | 42 | N08 | 45.52 |
| 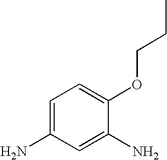 | 166.224 | 4712 | 14 | H10 | 45.55 |
| 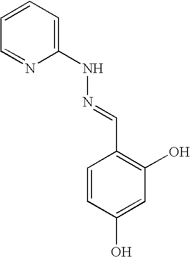 | 229.239 | 5831 | 17 | G14 | 45.71 |
| 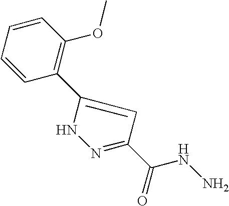 | 232.243 | 8337 | 24 | A17 | 46.18 |
| 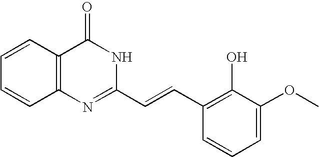 | 294.31 | 16440 | 47 | H17 | 46.66 |
| 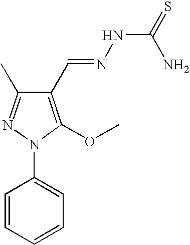 | 289.363 | 9698 | 28 | B14 | 46.91 |
| 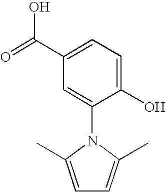 | 231.251 | 15644 | 45 | L11 | 47.17 |
| 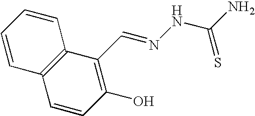 | 245.306 | 14774 | 42 | F23 | 47.58 |
| 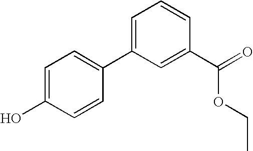 | 242.274 | 19114 | 55 | J08 | 47.74 |
| 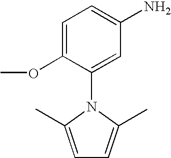 | 216.284 | 19013 | 55 | E02 | 48.09 |
| 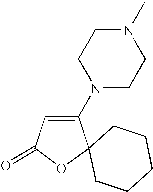 | 250.342 | 1203 | 4 | C11 | 48.47 |
| 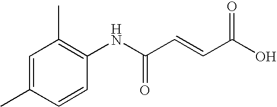 | 219.24 | 11955 | 34 | C23 | 48.60 |
| 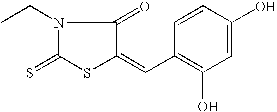 | 281.356 | 18027 | 52 | K06 | 48.81 |
| 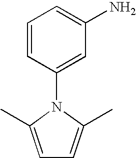 | 186.258 | 19011 | 55 | C02 | 48.96 |
| 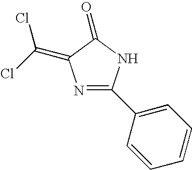 | 241.077 | 14683 | 42 | K17 | 49.03 |
| 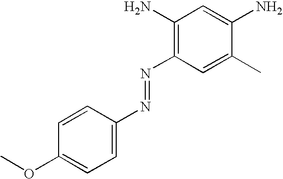 | 256.309 | 14087 | 41 | G02 | 49.14 |
| 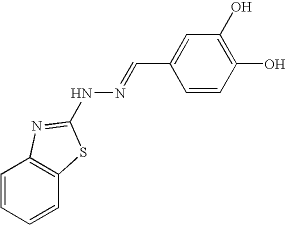 | 285.327 | 19031 | 55 | G03 | 49.49 |
| 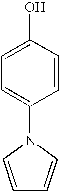 | 159.188 | 2889 | 9 | I06 | 49.74 |
| 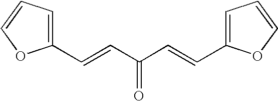 | 214.22 | 3546 | 11 | J03 | 50.00 |
| 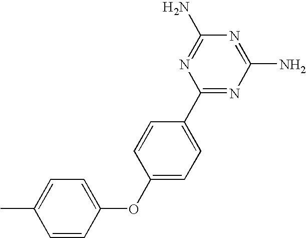 | 293.33 | 17633 | 51 | A04 | 50.27 |
| 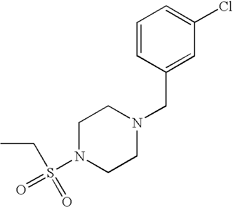 | 302.826 | 10898 | 31 | B23 | 50.32 |
| 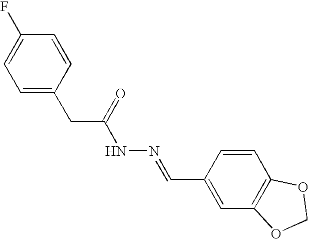 | 300.289 | 15092 | 43 | D21 | 50.40 |
| 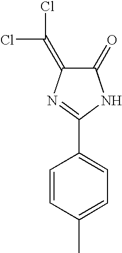 | 255.104 | 14478 | 42 | N04 | 50.87 |
| 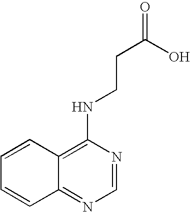 | 217.228 | 8107 | 24 | K02 | 51.21 |
| 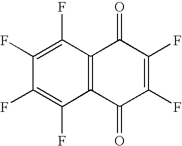 | 266.096 | 4896 | 14 | P21 | 51.38 |
| 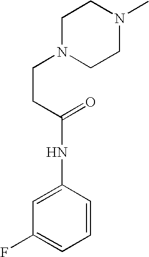 | 265.332 | 10882 | 31 | B22 | 51.65 |
| 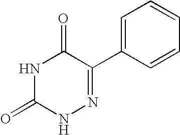 | 189.174 | 8099 | 24 | C02 | 52.20 |
| 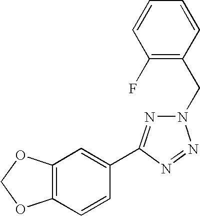 | 298.277 | 4123 | 12 | K17 | 52.35 |
| 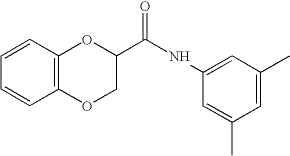 | 283.327 | 15109 | 43 | E22 | 52.72 |
| 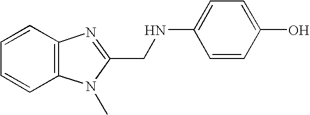 | 253.305 | 11396 | 33 | D10 | 53.12 |
| 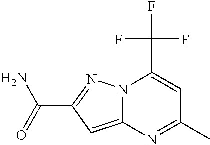 | 244.176 | 12642 | 36 | B22 | 53.16 |
| 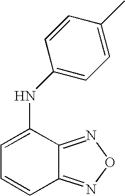 | 225.251 | 16409 | 47 | I15 | 53.26 |
| 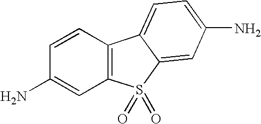 | 246.29 | 3484 | 10 | L21 | 53.37 |
| 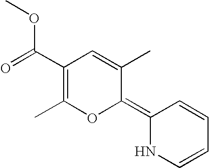 | 245.278 | 3434 | 10 | J18 | 53.53 |
| 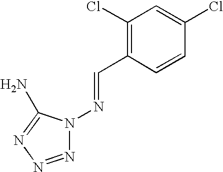 | 257.084 | 3530 | 11 | J02 | 53.82 |
| 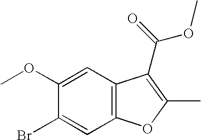 | 299.12 | 16504 | 47 | H21 | 54.16 |
| 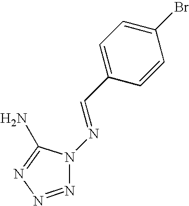 | 267.09 | 3531 | 11 | K02 | 54.31 |
| 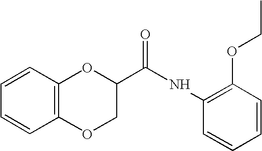 | 299.326 | 15108 | 43 | D22 | 54.41 |
| 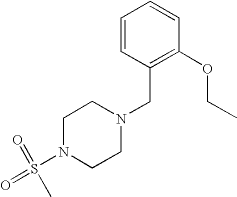 | 298.407 | 10911 | 31 | O23 | 54.49 |
| 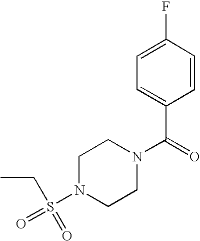 | 300.354 | 10912 | 31 | P23 | 54.57 |
| 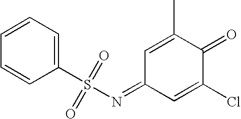 | 295.746 | 16827 | 48 | K19 | 54.59 |
| 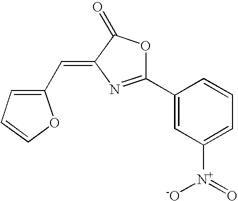 | 284.227 | 3535 | 11 | O02 | 55.05 |
| 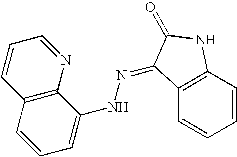 | 288.31 | 15060 | 43 | D19 | 55.18 |
| 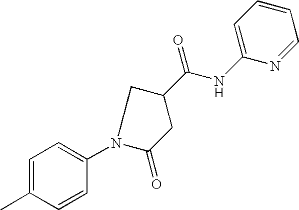 | 295.342 | 11425 | 33 | A12 | 55.62 |
| 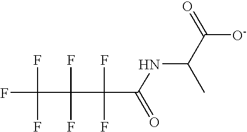 | 284.107 | 12639 | 36 | O21 | 55.75 |
| 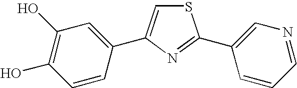 | 270.312 | 12357 | 36 | E04 | 55.79 |
| 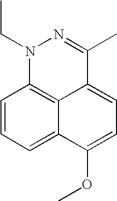 | 240.306 | 3448 | 10 | H19 | 56.36 |
| 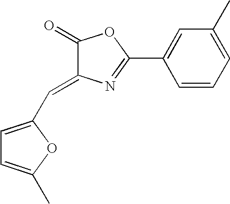 | 267.284 | 15126 | 43 | F23 | 56.46 |
| 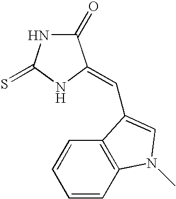 | 257.317 | 15134 | 43 | N23 | 56.50 |
| 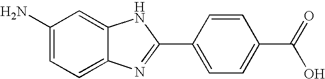 | 253.261 | 18318 | 53 | N02 | 56.68 |
| 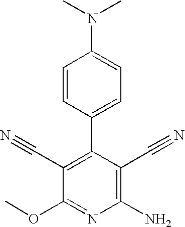 | 293.33 | 3524 | 11 | D02 | 56.69 |
| 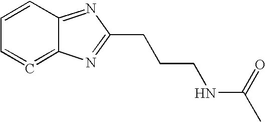 | 215.256 | 5067 | 15 | K10 | 56.74 |
| 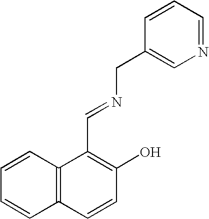 | 262.312 | 14438 | 42 | F02 | 56.74 |
| 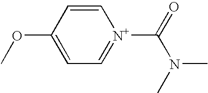 | 181.215 | 1075 | 4 | C03 | 56.79 |
| 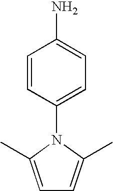 | 186.258 | 19010 | 55 | B02 | 56.83 |
| 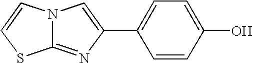 | 216.264 | 4042 | 12 | J12 | 56.90 |
| 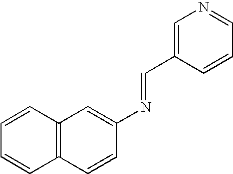 | 232.286 | 14634 | 42 | J14 | 56.91 |
| 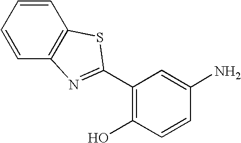 | 242.302 | 4051 | 12 | C13 | 57.06 |
| 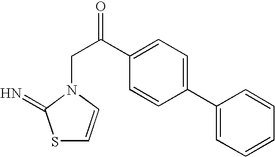 | 294.378 | 3525 | 11 | E02 | 57.10 |
| 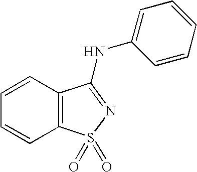 | 258.301 | 3534 | 11 | N02 | 57.10 |
| 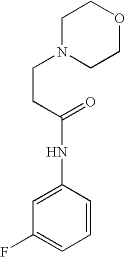 | 252.289 | 10881 | 31 | A22 | 57.20 |
| 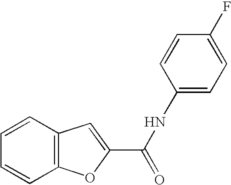 | 255.248 | 15111 | 43 | G22 | 57.42 |
| 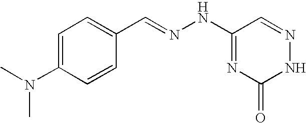 | 258.285 | 15122 | 43 | B23 | 57.54 |
| 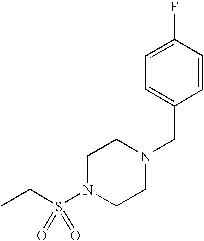 | 286.371 | 10901 | 31 | E23 | 57.54 |
| 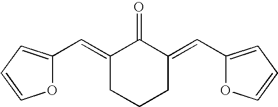 | 254.285 | 3521 | 11 | A02 | 57.59 |
| 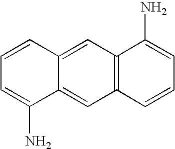 | 208.264 | 4409 | 13 | I13 | 57.72 |
| 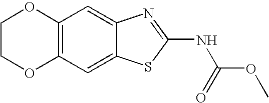 | 266.277 | 3414 | 10 | F17 | 57.75 |
| 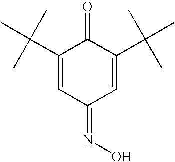 | 235.327 | 3527 | 11 | G02 | 57.75 |
| 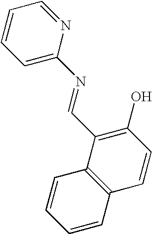 | 248.285 | 19617 | 56 | A18 | 58.00 |
| 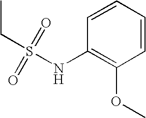 | 215.273 | 18809 | 54 | I11 | 58.09 |
| 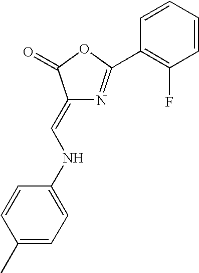 | 296.301 | 15125 | 43 | E23 | 58.54 |
| 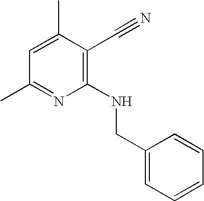 | 237.306 | 15132 | 43 | L23 | 58.81 |
| 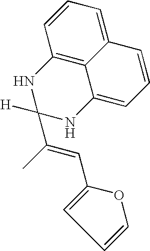 | 276.339 | 16054 | 46 | F15 | 58.84 |
| 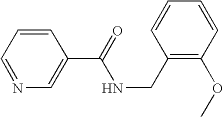 | 242.278 | 18289 | 52 | A23 | 58.89 |
| 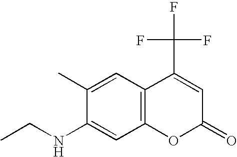 | 271.238 | 19916 | 57 | L14 | 58.97 |
| 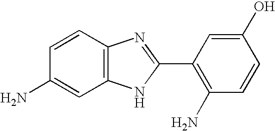 | 240.266 | 17129 | 49 | I16 | 58.99 |
| 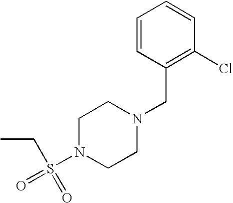 | 302.826 | 10897 | 31 | A23 | 59.00 |
| 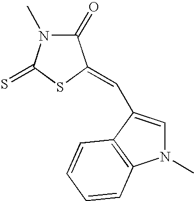 | 288.395 | 15135 | 43 | O23 | 59.01 |
| 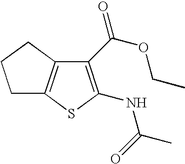 | 253.322 | 14419 | 41 | C23 | 59.07 |
| 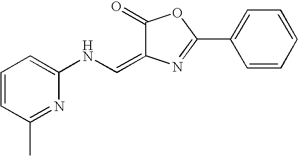 | 279.299 | 15123 | 43 | C23 | 59.16 |
| 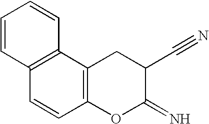 | 220.231 | 3526 | 11 | F02 | 59.23 |
| 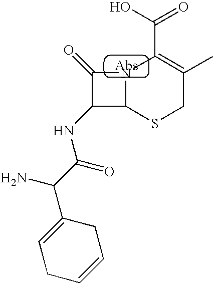 | 349.411 | 20044 | 58 | M14 | 59.29 |
| 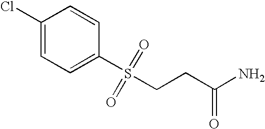 | 247.702 | 14129 | 41 | A05 | 59.35 |
| 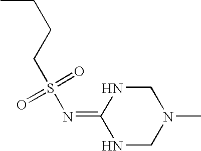 | 234.324 | 1267 | 4 | C15 | 59.49 |
| 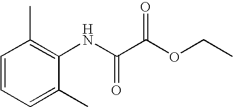 | 221.256 | 12671 | 36 | O23 | 59.68 |
| 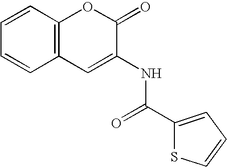 | 271.296 | 15124 | 43 | D23 | 59.70 |
| 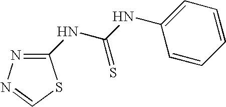 | 236.323 | 3523 | 11 | C02 | 59.80 |
| 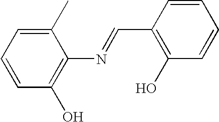 | 227.263 | 17889 | 51 | A20 | 59.85 |
| 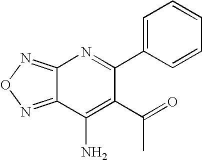 | 254.249 | 16312 | 47 | H09 | 59.86 |
| 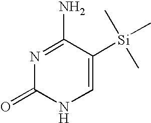 | 183.286 | 319 | 1 | O21 | 59.88 |
| 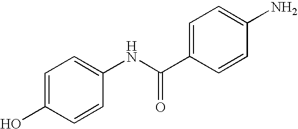 | 228.251 | 3353 | 10 | I13 | 60.08 |

| Structures | logP | logSw | H_acceptor | H_donor | B_rotN |
|---|---|---|---|---|---|
| 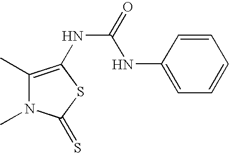 | 2.5046 | −4.26788 | 2 | 2 | 4 |
| 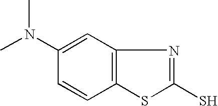 | 2.423 | −3.61712 | 1 | 0 | 1 |
| 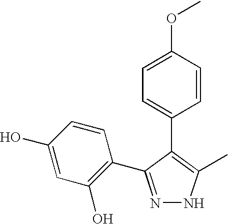 | 3.47192 | −4.28235 | 5 | 4 | 3 |
| 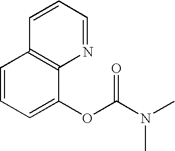 | 2.2952 | −2.05481 | 2 | 0 | 3 |
| 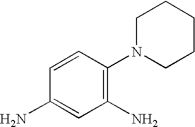 | 1.8413 | −1.5082 | 0 | 2 | 1 |
| 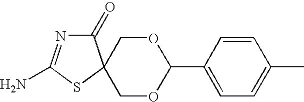 | 1.2348 | −4.10453 | 4 | 1 | 1 |
| 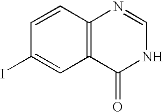 | 1.9657 | −3.07925 | 2 | 1 | 0 |
| 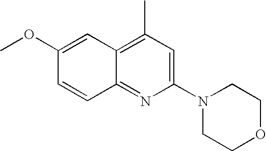 | 2.3884 | −3.02787 | 3 | 0 | 2 |
| 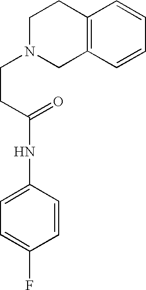 | 3.7503 | −3.61353 | 2 | 1 | 5 |
| 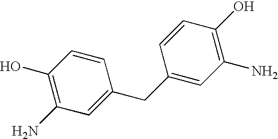 | 1.853 | −2.21912 | 2 | 4 | 2 |
| 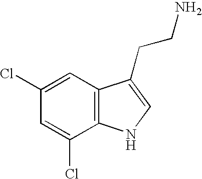 | 3.0285 | −2.86975 | 1 | 2 | 2 |
| 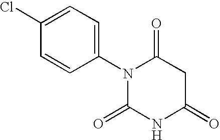 | 1.3392 | −2.86187 | 3 | 1 | 1 |
| 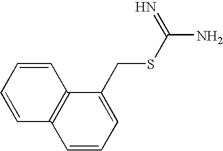 | 3.0049 | −4.76348 | 1 | 2 | 3 |
| 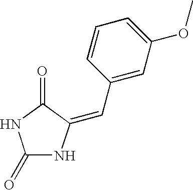 | 0.8755 | −2.53013 | 3 | 2 | 2 |
| 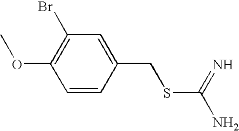 | 2.6961 | −4.02637 | 2 | 2 | 4 |
| 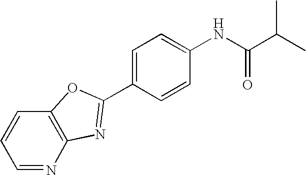 | 3.4843 | −5.76502 | 3 | 1 | 4 |
| 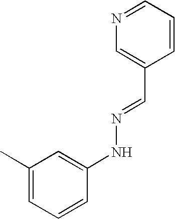 | 2.836 | −2.22269 | 2 | 1 | 3 |
| 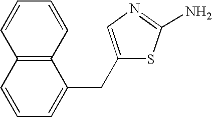 | 3.4693 | −4.41086 | 1 | 1 | 2 |
| 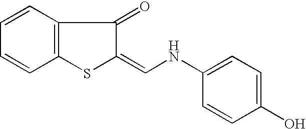 | 3.4061 | −3.83129 | 2 | 2 | 2 |
| 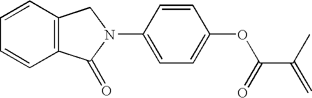 | 3.5949 | −4.23062 | 2 | 0 | 4 |
| 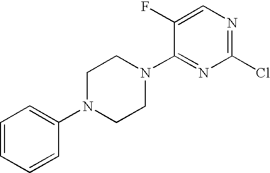 | 2.8933 | −4.02801 | 2 | 0 | 2 |
| 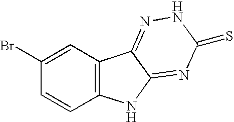 | 1.7106 | −4.86333 | 3 | 2 | 0 |
| 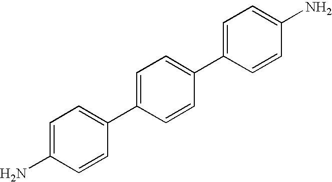 | 4.185 | −5.6794 | 0 | 2 | 2 |
| 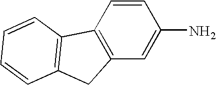 | 2.84 | −4.00302 | 0 | 1 | 0 |
| 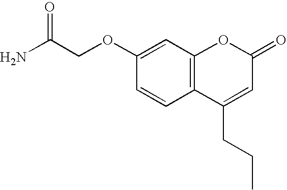 | 1.6532 | −4.46078 | 3 | 1 | 5 |
| 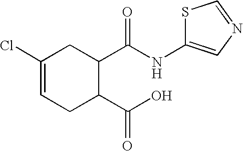 | 2.3151 | −1.52599 | 4 | 3 | 4 |
| 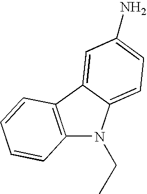 | 3.663 | −3.46356 | 0 | 1 | 1 |
| 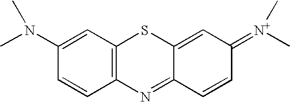 | 2.8696 | −3.86423 | 1 | 0 | 1 |
| 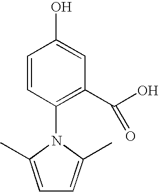 | 2.4979 | −1.7802 | 3 | 3 | 2 |
| 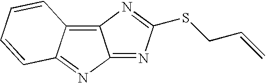 | 2.5801 | −4.61999 | 3 | 0 | 3 |
| 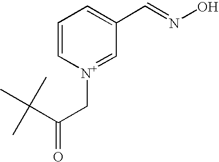 | 1.6638 | −0.76171 | 3 | 1 | 4 |
| 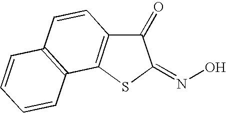 | 2.6879 | −4.34473 | 3 | 1 | 0 |
| 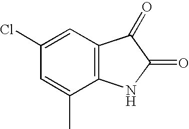 | 1.8095 | −2.93548 | 2 | 1 | 0 |
| 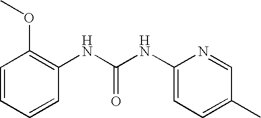 | 3.0426 | −2.51602 | 3 | 2 | 5 |
| 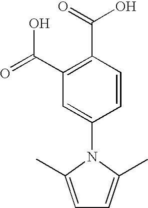 | 2.4905 | −2.11155 | 4 | 4 | 3 |
| 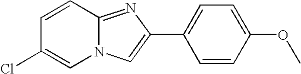 | 3.6227 | −3.89211 | 2 | 0 | 2 |
| 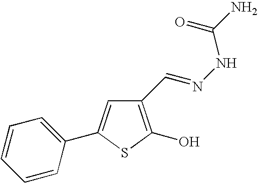 | 2.1229 | −3.7157 | 3 | 3 | 4 |
| 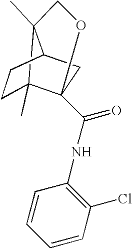 | 3.9001 | −4.46171 | 2 | 1 | 3 |
| 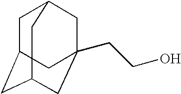 | 2.5852 | −4.24784 | 1 | 1 | 2 |
| 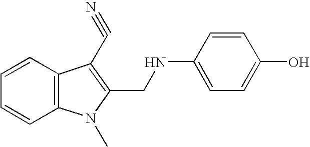 | 3.9933 | −3.27505 | 2 | 2 | 4 |
| 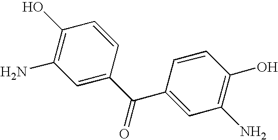 | 1.4932 | −2.17555 | 3 | 4 | 2 |
| 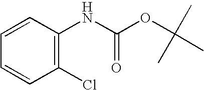 | 3.7133 | −3.37971 | 1 | 1 | 4 |
| 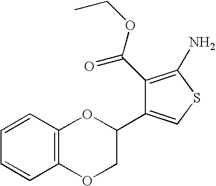 | 3.115 | −3.79721 | 3 | 1 | 4 |
| 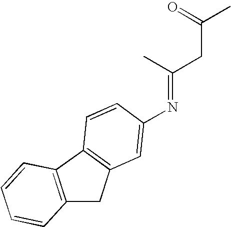 | 4.3293 | −4.96577 | 2 | 0 | 3 |
| 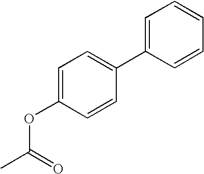 | 3.2789 | −4.16478 | 1 | 0 | 3 |
| 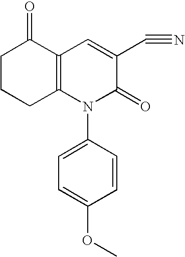 | 2.4989 | −3.59974 | 4 | 0 | 3 |
| 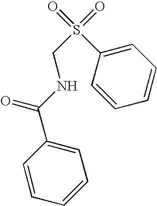 | 1.8478 | −3.17154 | 3 | 1 | 5 |
| 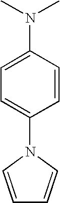 | 2.5433 | −1.47338 | 0 | 0 | 2 |
| 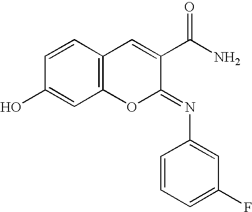 | 2.7939 | −4.91081 | 3 | 2 | 2 |
| 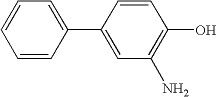 | 2.6414 | −3.17019 | 1 | 2 | 1 |
| 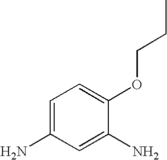 | 1.6398 | −1.406 | 1 | 2 | 3 |
| 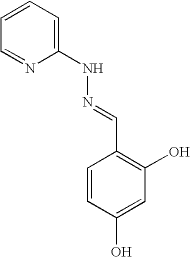 | 1.9388 | −1.33613 | 4 | 3 | 3 |
| 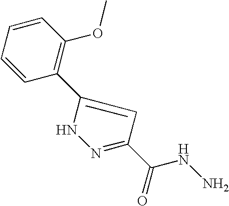 | 0.6888 | −2.65145 | 5 | 4 | 4 |
| 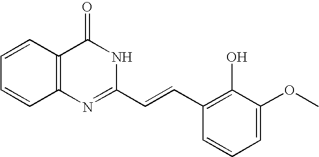 | 2.8876 | −4.1536 | 4 | 2 | 3 |
| 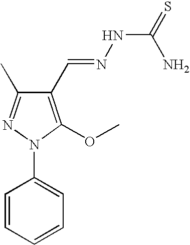 | 1.1284 | −3.3999 | 4 | 2 | 5 |
| 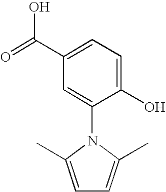 | 2.4979 | −1.7802 | 3 | 3 | 2 |
| 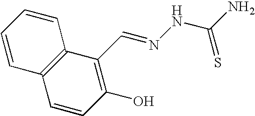 | 1.4845 | −4.08689 | 3 | 3 | 3 |
| 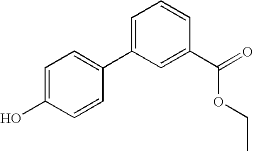 | 3.2359 | −4.15825 | 2 | 1 | 4 |
| 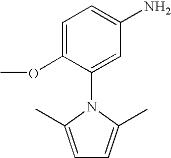 | 2.6849 | −1.94401 | 1 | 1 | 2 |
| 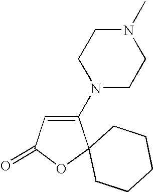 | 1.3774 | −2.13468 | 2 | 0 | 1 |
| 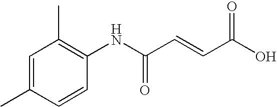 | 1.8827 | −2.72302 | 3 | 3 | 4 |
| 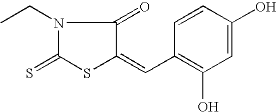 | 1.8629 | −3.84126 | 4 | 2 | 2 |
| 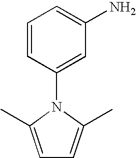 | 2.6763 | −1.89363 | 0 | 1 | 1 |
| 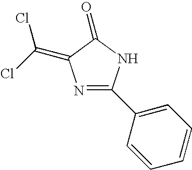 | 2.2097 | −4.38537 | 2 | 1 | 1 |
| 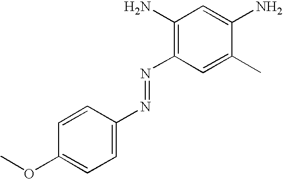 | 3.5834 | −2.93029 | 3 | 2 | 3 |
| 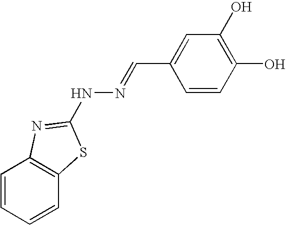 | 3.1535 | −3.56986 | 4 | 3 | 3 |
| 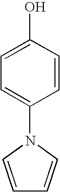 | 2.1829 | −1.18402 | 1 | 1 | 1 |
| 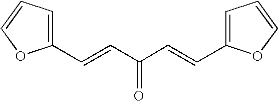 | 3.1683 | −3.97247 | 1 | 0 | 4 |
| 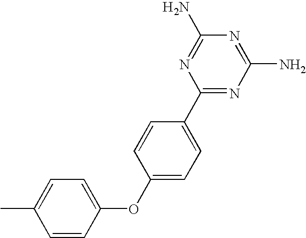 | 2.8037 | −6.03773 | 4 | 2 | 3 |
| 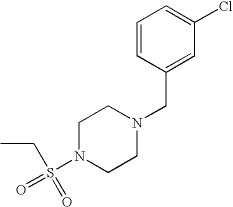 | 2.1 | −2.35071 | 3 | 0 | 4 |
| 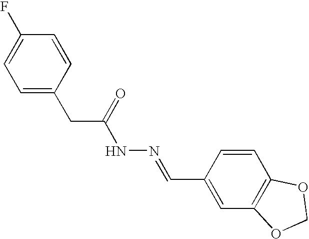 | 2.5185 | −3.8497 | 4 | 1 | 5 |
| 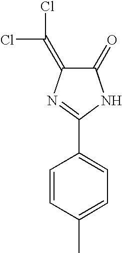 | 2.5181 | −4.85929 | 2 | 1 | 1 |
| 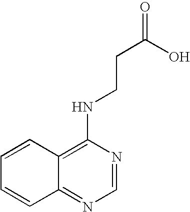 | 1.5164 | −2.1199 | 4 | 3 | 4 |
| 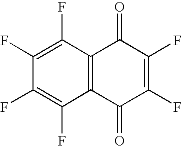 | 3.8578 | −4.60776 | 2 | 0 | 0 |
| 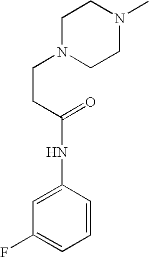 | 1.673 | −1.70849 | 3 | 1 | 5 |
| 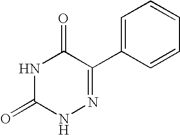 | 0.2301 | −2.44642 | 3 | 2 | 1 |
| 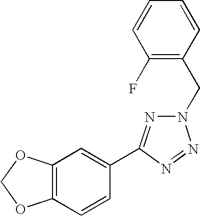 | 2.7939 | −3.93311 | 5 | 0 | 3 |
| 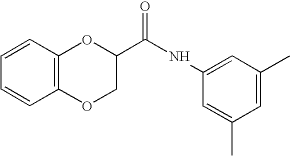 | 3.0819 | −4.57875 | 3 | 1 | 3 |
| 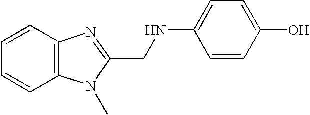 | 3.5166 | −2.73282 | 2 | 2 | 3 |
| 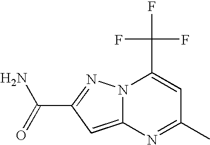 | 1.4912 | −2.69743 | 3 | 1 | 2 |
| 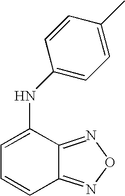 | 3.2748 | −3.93377 | 2 | 1 | 2 |
| 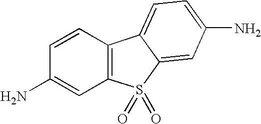 | 1.6642 | −3.57207 | 2 | 2 | 0 |
| 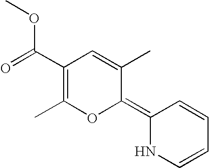 | 2.2946 | −2.9308 | 2 | 1 | 2 |
| 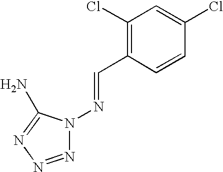 | 1.4969 | −3.02781 | 4 | 1 | 2 |
| 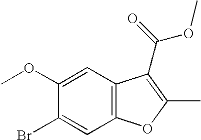 | 3.3722 | −4.6661 | 2 | 0 | 3 |
| 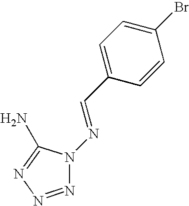 | 0.9733 | −2.64962 | 4 | 1 | 2 |
| 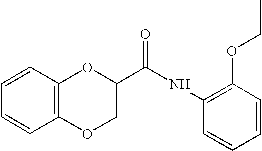 | 2.8638 | −4.0085 | 4 | 1 | 5 |
| 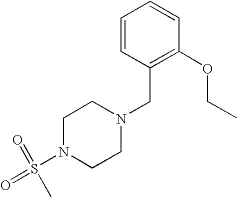 | 1.4289 | −1.6668 | 4 | 0 | 5 |
| 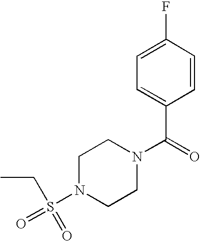 | 1.2045 | −2.08009 | 3 | 0 | 4 |
| 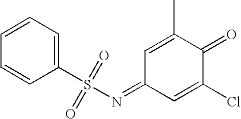 | 2.468 | −4.40322 | 4 | 0 | 2 |
| 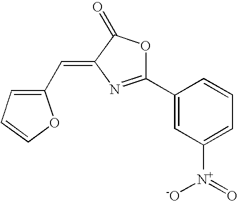 | 2.5323 | −5.59881 | 2 | 0 | 3 |
| 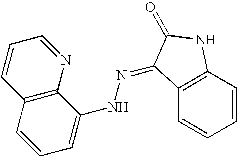 | 3.0031 | −4.24518 | 3 | 2 | 2 |
| 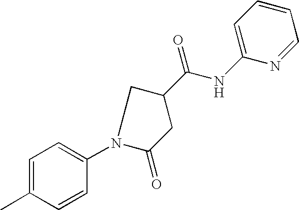 | 2.3816 | −2.79169 | 3 | 1 | 4 |
| 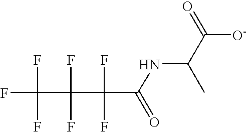 | 0.0739 | −2.83628 | 1 | 1 | 6 |
| 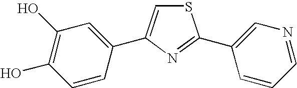 | 3.2833 | −3.35331 | 4 | 2 | 2 |
| 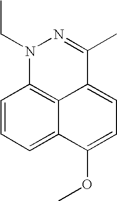 | 3.4123 | −3.99862 | 2 | 0 | 2 |
| 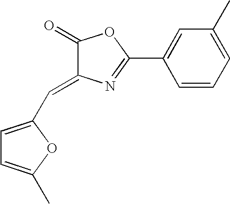 | 3.2409 | −5.59589 | 2 | 0 | 2 |
| 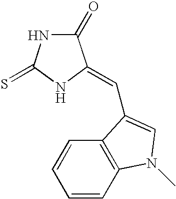 | 1.6547 | −3.89862 | 2 | 2 | 1 |
| 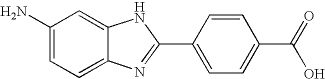 | 2.5103 | −4.18052 | 4 | 5 | 2 |
| 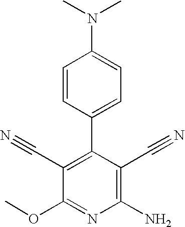 | 2.1488 | −3.57617 | 4 | 1 | 5 |
| 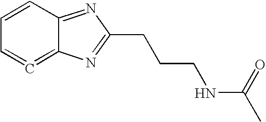 | 1.3646 | −2.02418 | 3 | 1 | 5 |
| 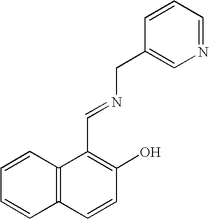 | 3.8259 | −3.51145 | 3 | 1 | 3 |
| 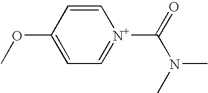 | 0.5124 | −0.16928 | 2 | 0 | 3 |
| 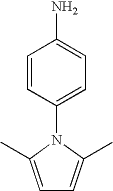 | 2.6763 | −1.89363 | 0 | 1 | 1 |
| 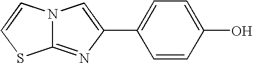 | 2.5616 | −3.13193 | 2 | 1 | 1 |
| 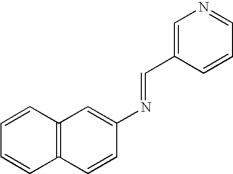 | 3.9854 | −3.92936 | 2 | 0 | 2 |
| 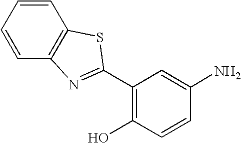 | 3.2511 | −4.11458 | 2 | 2 | 1 |
| 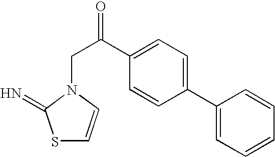 | 3.7631 | −5.0238 | 2 | 1 | 4 |
| 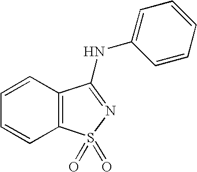 | 2.2476 | −3.85109 | 3 | 1 | 2 |
| 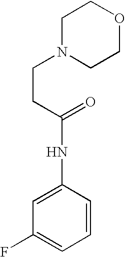 | 1.7578 | −1.95324 | 3 | 1 | 5 |
| 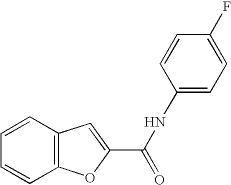 | 4.0955 | −5.2481 | 1 | 1 | 3 |
| 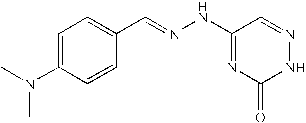 | 0.7836 | −2.40904 | 4 | 2 | 4 |
| 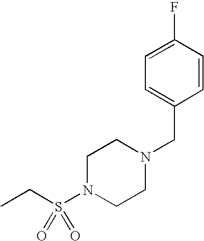 | 1.8307 | −1.9114 | 3 | 0 | 4 |
| 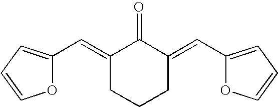 | 4.0926 | −4.41955 | 1 | 0 | 2 |
| 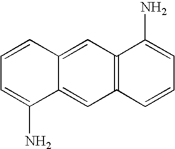 | 3.1574 | −4.5824 | 0 | 2 | 0 |
| 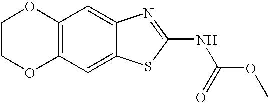 | 2.2458 | −3.20024 | 4 | 1 | 3 |
| 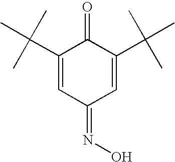 | 3.3443 | −4.35416 | 3 | 1 | 2 |
| 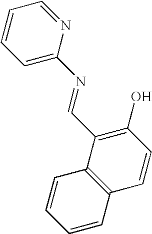 | 3.691 | −3.87867 | 3 | 1 | 2 |
| 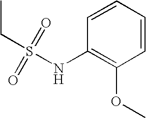 | 1.4568 | −1.60045 | 3 | 1 | 4 |
| 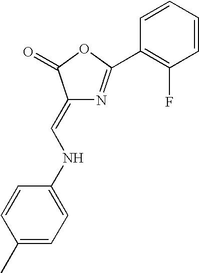 | 3.6623 | −5.41556 | 2 | 1 | 3 |
| 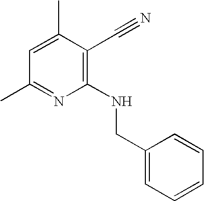 | 3.4486 | −3.1406 | 2 | 1 | 4 |
| 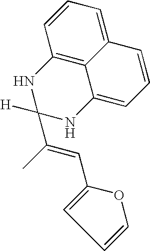 | 4.6998 | −4.92749 | 0 | 2 | 2 |
| 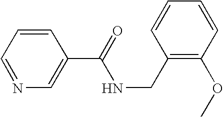 | 2.2866 | −2.09115 | 3 | 1 | 5 |
| 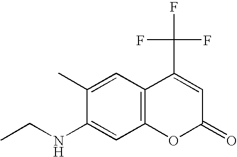 | 3.2915 | −4.14772 | 1 | 1 | 3 |
| 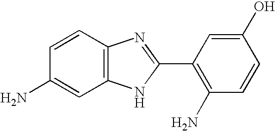 | 2.0999 | −3.57005 | 3 | 5 | 1 |
| 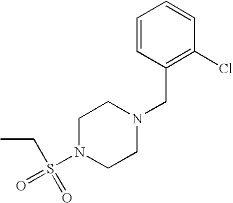 | 2.1 | −2.35071 | 3 | 0 | 4 |
| 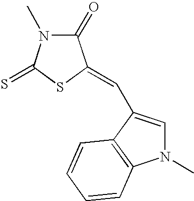 | 2.9125 | −4.42186 | 2 | 0 | 1 |
| 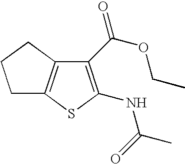 | 2.3718 | −2.84187 | 2 | 1 | 5 |
| 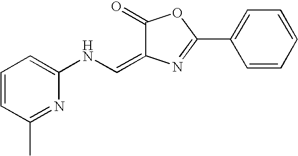 | 2.6469 | −4.01317 | 3 | 1 | 3 |
| 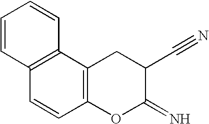 | 3.1165 | −5.2366 | 2 | 1 | 1 |
| 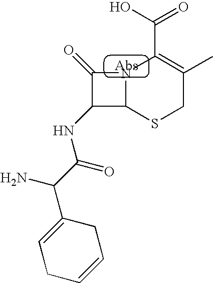 | 0.1206 | −2.52286 | 5 | 4 | 5 |
| 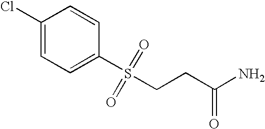 | 1.0154 | −2.42034 | 3 | 1 | 4 |
| 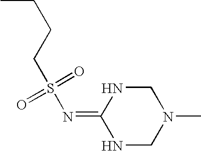 | −0.4881 | −0.42412 | 4 | 2 | 4 |
| 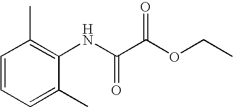 | 1.805 | −2.64329 | 2 | 1 | 5 |
| 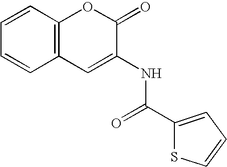 | 2.438 | −4.58369 | 2 | 1 | 3 |
| 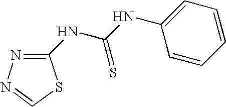 | 2.119 | −4.4069 | 3 | 2 | 4 |
| 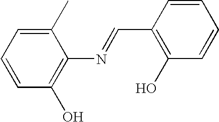 | 3.1568 | −2.74619 | 3 | 2 | 2 |
| 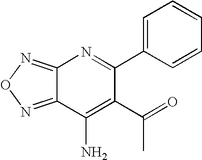 | 2.0696 | −4.24678 | 4 | 1 | 2 |
| 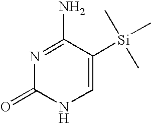 | 0.8281 | −0.30668 | 2 | 2 | 1 |
| 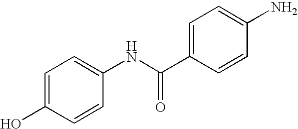 | 2.2267 | −2.7138 | 2 | 3 | 3 |

TABLE B
Inhibitors of the PKA-RIIα-GST-AKAP 185 interaction (substance library FMP20000 (ChemDiv))

| | ID_No. | | | MW | IC50/μM |
|---|---|---|---|---|---|
| Comp_ID | ChemDiv | Struktur | Formula | g/mol | RIIa-18d |
| 14741 | 1602-1355 | 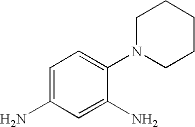 | C11H17N3 | 191.27 | 10 |
| 990 | 1749-0062 | 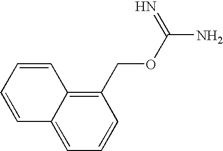 | C12H12N2S | 216.30 | 13 |
| 19753 | 3237-0008 | 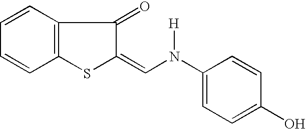 | C15H11NO2S | 269.32 | 21 |
| 18882 | 1731-0077 | 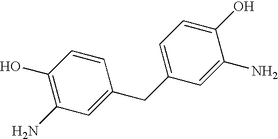 | C13H14N2O2 | 230.26 | 25 |
| 11546 | K072- 0237 | 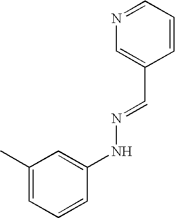 | C13H13N3 | 211.26 | 29 |
| 8724 | 3335-0360 | 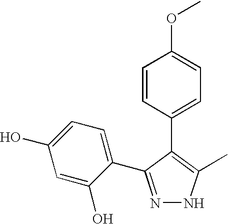 | C17H16N2O3 | 296.32 | 39 |
| 14794 | 1682-5682 | 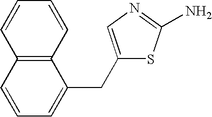 | C14H12N2S | 240.32 | 54 |
| 19814 | 3630-0129 | 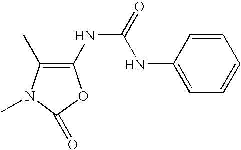 | C12H13N3OS2 | 279.38 | 71 |
| 4496 | 8010-0255 | 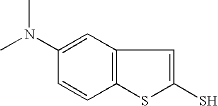 | C9H10N2S2 | 210.32 | 75 |

| | | | | | | | | | Bindung |
|---|---|---|---|---|---|---|---|---|---|
| Comp_ID | Plate_ID | Pos_ID | Barcode | logP | logSw | H_acceptor | H_donor | B_rotN | % |
| 14741 | 42 | E21 | 402000042 | 1.416 | −196.629 | 0 | 4 | 1 | 2.3 |
| 990 | 3 | N19 | 402000003 | 3.440 | −122.247 | 2 | 3 | 3 | 12.4 |
| 19753 | 57 | I04 | 402000057 | 2.491 | −34.951 | 3 | 2 | 3 | 19.3 |
| 18882 | 54 | B16 | 402000054 | 1.777 | −254.994 | 2 | 6 | 4 | 3.5 |
| 11546 | 33 | J19 | 402000033 | 2.342 | −310.178 | 2 | 1 | 3 | 16.4 |
| 8724 | 25 | D19 | 402000025 | 3.334 | −294.296 | 4 | 3 | 5 | 1.3 |
| 14794 | 43 | J02 | 402000043 | 3.164 | −34.827 | 2 | 2 | 2 | 17.7 |
| 19814 | 57 | F08 | 402000057 | 3.314 | −260.326 | 3 | 2 | 4 | 0.3 |
| 4496 | 13 | P18 | 402000013 | 3.308 | −268.578 | 3 | 1 | 2 | 0.6 |

TABLE C
Derivatives of substance 990

| Sub- | | | |
|---|---|---|---|
| stance | Structure | Formula | MW |
| SM13 | 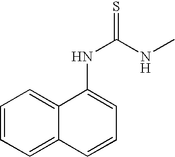 | C12H12N2S | 216.3 |
| SM25 | 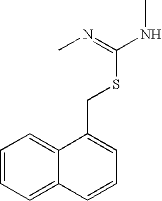 | C14H16N2S | 244.36 |
| SM33 | 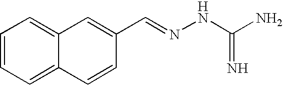 | C12H12N4 | 212.25 |
| SM39 | 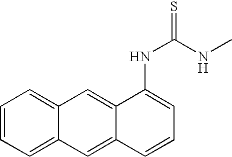 | C14H16N2S | 266.36 |
| SM42 | 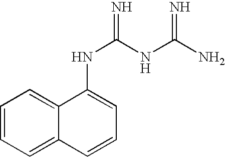 | C12H13N5 | 227.27 |
| SM44 | 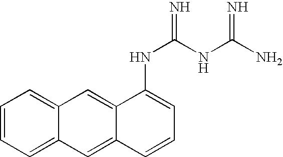 | C16H15N5 | 277.32 |
| SM47 | 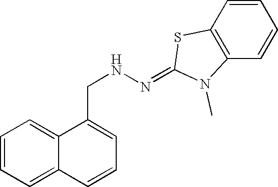 | C19H17N3S | 319.42 |
| SM51 | 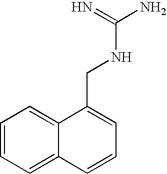 | C12H13N3 | 199.25 |
| SM52 | 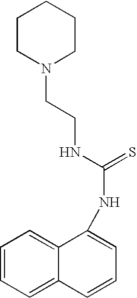 | C18H23N3S | 313.46 |
| SM53 | 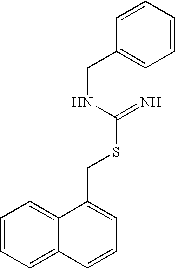 | C19H18N2S | 306.42 |
| SM54 | 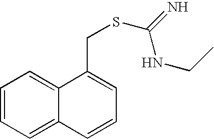 | C14H16N2S | 244.36 |
| SM55 | 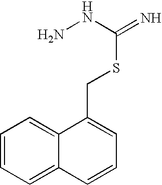 | C12H13N3S | 231.32 |
| SM56 | 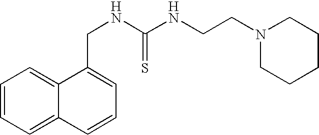 | C19H25N3S | 327.49 |
| SM57 | 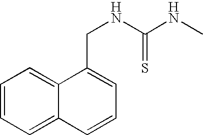 | C13H14N2S | 230.33 |
| SM58 | 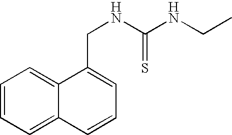 | C14H16N2S | 244.36 |
| SM59 | 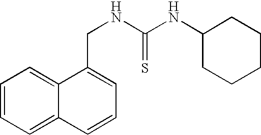 | C18H22N2S | 298.45 |
| SM61 | 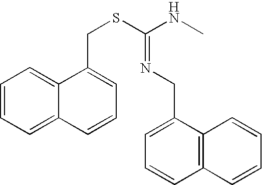 | C24H22N2S | 370.51 |
| SM63 | 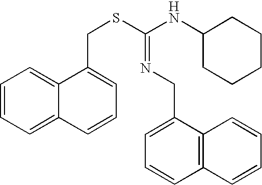 | C29H30N2S | 438.63 |
| SM65 | 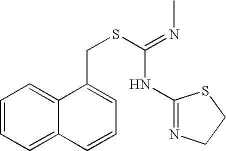 | C16H17N3S2 | 315.46 |
| SM66 | 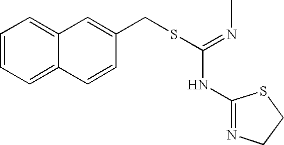 | C16H17N3S2 | 315.46 |
| SM67 | 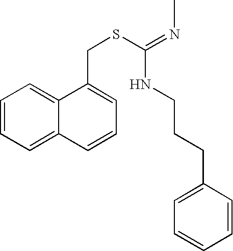 | C22H24N2S | 348.5 |
| SM68 | 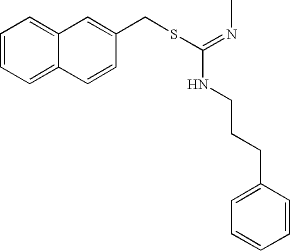 | C22H24N2S | 348.5 |
| SM69 | 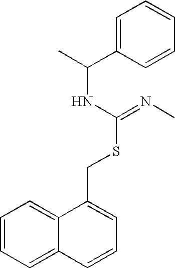 | C21H22N2S | 334.48 |
| SM70 | 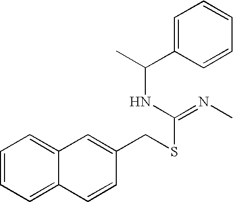 | C21H22N2S | 334.15 |
| SM71 | 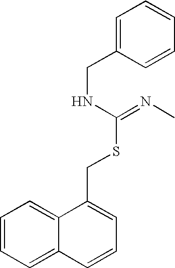 | C20H20N2S | 320.45 |
| SM72 | 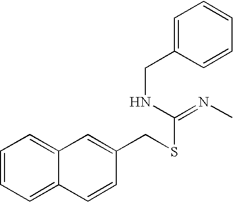 | C20H20N2S | 320.45 |
| JG5 | 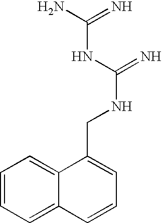 | C13H15N5 | 241.29 |
| JG20 | 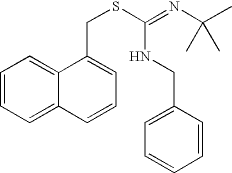 | C23H26N2S | 362.53 |
| JG21 | 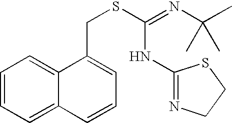 | C19H23N3S2 | 357.54 |
| JG23 | 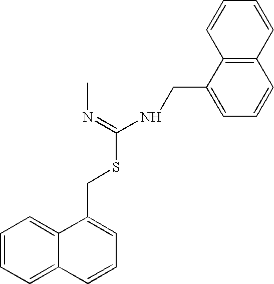 | C24H22N2S | 370.51 |
| JG24 | 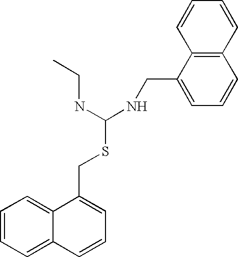 | C25H24N2S | 384.54 |
| JG25 | 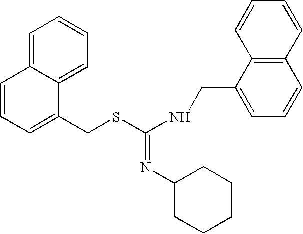 | C29H30N2S | 438.63 |
| JG26 | 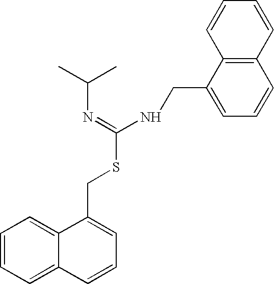 | C26H26N2S | 398.56 |
| JG27 | 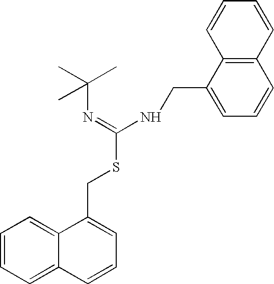 | C27H28N2S | 412.59 |
| JG28 | 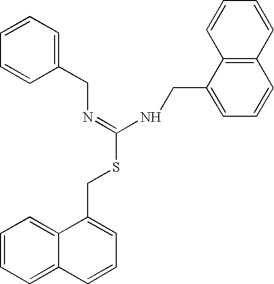 | C30H26N2S | 446.61 |
| JG31 | 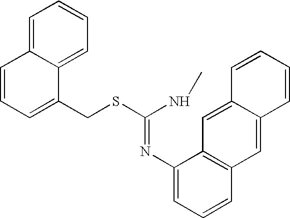 | C27H22N2S | 406.15 |

| | | | | | | | Ratio | | | |
|---|---|---|---|---|---|---|---|---|---|---|
| | | | | | | | IC₅₀ (comp./990) | | | |
| | | | | IC₅₀ in Screening | | | in Screening no. 1-3 | | | Mean |
| Sub- | | H- | H- | no. 1-3 (μM) | | | (μM) | | | IC₅₀ |
| stance | logP | Acc. | Don. | 1 | 2 | 3 | 1 | 2 | 3 | (μM) |
| SM13 | 2.055 | 2 | 2 | — | | | | | | |
| SM25 | 2.943 | 2 | 1 | 0.64 | 0.76 | 0.21 | 0.97 | 1.55 | 0.68 | 0.52 |
| SM33 | 2.349 | 4 | 4 | — | | | | | | |
| SM39 | 3.229 | 2 | 2 | — | | | | | | |
| SM42 | 2.089 | 5 | 6 | — | | | | | | |
| SM44 | 3.263 | 5 | 6 | — | | | | | | |
| SM47 | 6.88 | 3 | 1 | — | | | | | | |
| SM51 | 1.438 | 3 | 4 | | 43.3 | | | 88.35 | | 43.00 |
| SM52 | 3.66 | 3 | 2 | | 472 | | | 962.65 | | 468.49 |
| SM53 | 4.842 | 2 | 2 | | 1.57 | 0.35 | | 3.20 | 1.13 | 1.05 |
| SM54 | 3.603 | 2 | 2 | | 3.15 | | | 6.43 | | 3.13 |
| SM55 | 2.134 | 3 | 4 | | 1.2 | | | 2.45 | | 1.19 |
| SM56 | 4.135 | 3 | 2 | | 290 | | | 592.04 | | 288.13 |
| SM57 | 2.514 | 2 | 2 | | 2.04 | | | 4.16 | | 2.03 |
| SM58 | 3.043 | 2 | 2 | | 483 | | | 986.12 | | 479.91 |
| SM59 | 4.545 | 2 | 2 | | 169 | | | 343.88 | | 167.35 |
| SM61 | 5.885 | 2 | 1 | | 0.08 | 0.1 | | 0.16 | 0.32 | 0.12 |
| SM63 | 7.916 | 2 | 1 | | 0.27 | 0.35 | 0.00 | 0.55 | 1.13 | 0.27 |
| SM65 | 1.68 | 3 | 1 | | 0.07 | 0.12 | | 0.14 | 0.39 | 0.13 |
| SM66 | 1.68 | 3 | 1 | | 14.8 | | | 30.29 | | 14.74 |
| SM67 | 5.419 | 2 | 1 | | 0.25 | 0.34 | | 0.51 | 1.10 | 0.39 |
| SM68 | 5.419 | 2 | 1 | | 42.4 | | | 86.51 | | 42.10 |
| SM69 | 5.02 | 2 | 1 | | 0.86 | | | 1.76 | | 0.85 |
| SM70 | 5.02 | 2 | 1 | | 39.1 | | | 79.84 | | 38.85 |
| SM71 | 4.711 | 2 | 1 | | 0.2 | 0.14 | | 0.41 | 0.45 | 0.21 |
| SM72 | 4.711 | 2 | 1 | | 27.1 | | | 55.39 | | 26.96 |
| JG5 | 0.158 | 5 | 6 | | | — | | | | |
| JG20 | 5.948 | 2 | 1 | | | 0.23 | | | 0.74 | 0.36 |
| JG21 | 2.917 | 3 | 1 | | | 0.61 | | | 1.97 | 0.96 |
| JG23 | 5.885 | 2 | 1 | | | bad | | | | |
| JG24 | 6.414 | 2 | 1 | | | | | | | |
| JG25 | 7.916 | 2 | 1 | | | 0.08 | | | 0.26 | 0.13 |
| JG26 | 6.723 | 2 | 1 | | | 0.27 | | | 0.87 | 0.42 |
| JG27 | 7.122 | 2 | 1 | | | 0.32 | | | 1.03 | 0.50 |
| JG28 | 7.653 | 2 | 1 | | | 0.12 | | | 0.39 | 0.19 |
| JG31 | 6.726 | 2 | 1 | | | 2.57 | | | 8.29 | 4.03 |

TABLE 4

| Substance | Structure | Formula |
|---|---|---|
| SM-FP | 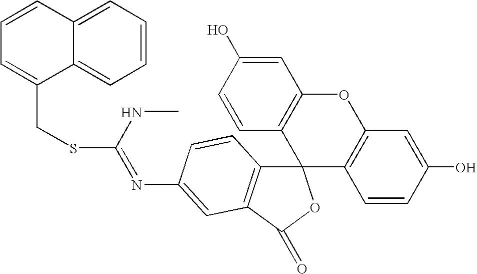 | C33H24N2O5S |
| 990 | 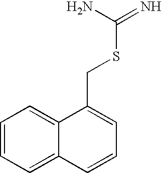 | C12H12N2S |

| | | | | | | | | Radio | | | |
|---|---|---|---|---|---|---|---|---|---|---|---|
| | | | | | | | | IC₅₀ (comp./990) | | | |
| | | | | | IC₅₀in Screening | | | in Screening no. 1-3 | | | Mean |
| | | | H- | H- | no. 1-3 (μM) | | | (μM) | | | IC50 |
| Substance | MW | logP | Acc. | Don. | 1 | 2 | 3 | 1 | 2 | 3 | (μM) |
| SM-FP | 560.62 | 4.891 | 7 | 3 | | | 0.17 | | | 0.55 | 0.27 |
| 990 | 216.3 | 3.44 | 2 | 3 | 0.66 | 0.49 | 0.31 | 1.00 | 1.00 | 1.00 | 0.49 |

TABLE D
Fluorescent dye-coupled substance SM-FP (derivative of substance 990)

| Substance | Structure | Formula |
|---|---|---|
| SM-FP | 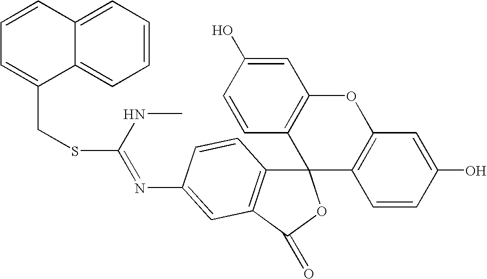 | C33H24N2O5S |
| 990 | 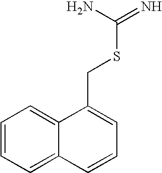 | C12H12N2S |

| | | | | | | | | Radio | | | |
|---|---|---|---|---|---|---|---|---|---|---|---|
| | | | | | | | | IC₅₀ (comp./990) | | | |
| | | | | | IC₅₀in Screening | | | in Screening no. 1-3 | | | Mean |
| | | | H- | H- | no. 1-3 (μM) | | | (μM) | | | IC50 |
| Substance | MW | logP | Acc. | Don. | 1 | 2 | 3 | 1 | 2 | 3 | (μM) |
| SM-FP | 560.62 | 4.891 | 7 | 3 | | | 0.17 | | | 0.55 | 0.27 |
| 990 | 216.3 | 3.44 | 2 | 3 | 0.66 | 0.49 | 0.31 | 1.00 | 1.00 | 1.00 | 0.49 |

1. Non-peptidic protein kinase A/protein kinase A anchor protein decouplers in accordance with Table A.

2. Non-peptidic protein kinase A/protein kinase A anchor protein decouplers having a molecular weight in the range of from 150 to 600 g/mol, preferably from 190 to 300 g/mol, a partition coefficient of log P≦10, preferably ≦8, with a maximum of 10 hydrogen bridge donors and a maximum of 10 hydrogen bridge acceptors, a solubility value log Sw of from −400 to 0, and a BrotN value of from 0 to 7.

3. The decouplers according to claim 2,
characterized in that
they have a maximum of 7, preferably 6H bridge donors, a maximum of 6, preferably 5, more preferably 4 hydrogen bridge acceptors and/or a log P value of ≧1 to ≦8, preferably ≧1 to ≦5.

4. The decouplers according to claim 2,
characterized in that
they inhibit the interaction of AKAP and PKA subunits by at least 40%, preferably by at least 80%.

5. The decouplers according to claim 1, said decouplers being selected from Table B.

6. The decouplers according to claim 1, said decouplers being selected from Table C.

7. The decouplers according to claim 1, said decouplers being selected from Table D.

8. The decouplers according to claim 1, in accordance with general formula I, said decouplers being interconvertible via mesomerism (R² and R³ are regarded as interchangeable) 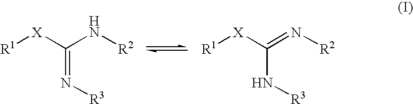
wherein
X is a non-hydrogen atom, preferably a sulfur atom,
R¹ is an alkyl or aryl residue, preferably a 1-naphthylmethyl residue,
R² and R³ are hydrogen atoms or alkyl or aryl residues, and R² and R³ are preferably two hydrogen atoms, two methyl residues, one benzyl residue and one methyl residue or one benzyl residue and one tert-butyl residue,
in a particularly preferred fashion, R² and R³ are a 2-thiazolidinyl residue and a methyl or tert-butyl residue, as well as a 1-naphthyl residue and an isopropyl, cyclohexyl, benzo or methyl residue; or in accordance with general formula II 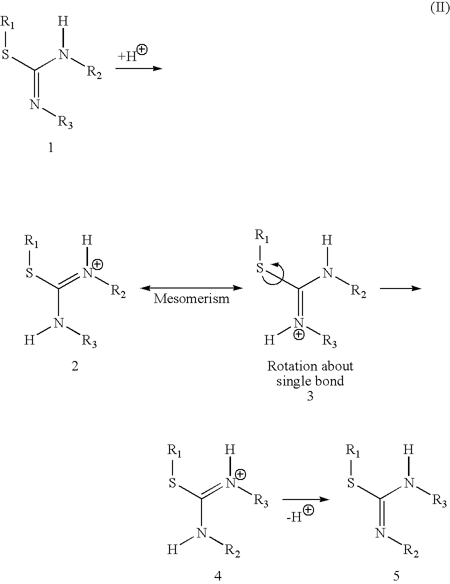
wherein X, R¹, R² and R³ have the same meaning as in formula I.

9. The decouplers according to claim 1, characterized in that
they comprise a structure in accordance with FIG. 19.

10. The decouplers according to claim 1, characterized in that
they comprise a structure in accordance with FIG. 20.

11. The decouplers according to claim 1, characterized in that
binding of AKAP18 proteins, preferably AKAP18delta proteins and/or RI alpha and/or RIIalpha and/or RI beta and/or RIIbeta is inhibited.

12. The decouplers according to claim 1 for the surgical and/or therapeutic treatment of a human or animal body and/or for use in diagnostic methods carried out on a human or animal body.

13. A pharmaceutical agent comprising at least one decoupler according to claim 1, additionally comprising at least one pharmaceutical carrier and/or adjuvants.

14. The pharmaceutical agent according to claim 1, characterized in that
the carrier is selected from the group comprising fillers, disintegrants, binders, humectants, extenders, dissolution retarders, absorption enhancers, wetting agents, adsorbents and/or lubricants.

15. The pharmaceutical agent according to claim 14, characterized in that
said agent is a capsule, a tablet, a coated tablet, a suppository, an ointment, a cream, a pad, an injection solution and/or an infusion solution.

16. A recognition molecule targeted to the decoupler according to claim 1, said recognition molecule being an antibody, a complexing agent and/or a chelating agent.

17. A kit comprising a decoupler according to claim 1, optionally together with information relating to combining and/or handling the components of the kit.

18. Method of using the decoupler and/or pharmaceutical agent and/or of the recognition molecule comprising the decoupler of claim 1 for the modification, particularly inhibition, of an AKAP-PKA interaction.

19. The method of claim 18, characterized in that
modifying the interaction is effected in a cell, a cell culture, a tissue and/or a target organism.

20. The method of claim 19, characterized in that
the modification of the interaction, the vasopressin-induced redistribution of AQP2 is modified, particularly prevented.

21. The method of claim 18, characterized in that
the interaction of RIalpha, RIIalpha, RIbeta and/or RIIbeta PKA subunits with AKAP is modified, particularly inhibited.

22. The method of claim 18, characterized in that
the agents according to claims 1 to 17 bind in a specific fashion to AKAP, preferably AKAP18, more preferably AKAP18delta and/or in a specific fashion to PKA, preferably to subunits thereof, and especially preferably to RII subunits.

23. The method of claim 18, characterized in that
the subunits are of human and/or murine origin and/or obtained from rats.

24. Method for exerting influence on a preferably compartmentalized cAMP-dependent signal transduction in vitro or in vivo via the decoupler or pharmaceutical agent comprising the decoupler of claim 1 to exert influence on a preferably compartmentalized cAMP-dependent signal transduction.

25. Production of a medicament for prophylaxis or treatment of diseases associated with defects of compartmentalized cAMP-dependent signal transduction comprising the decoupler of claim 1, wherein the disease is selected from the group comprising any type of asthma, etiology or pathogenesis, or asthma from the group of atopic asthma, non-atopic asthma, allergic asthma, IgE-mediated atopic asthma, bronchial asthma, essential asthma, primary asthma, endogenous asthma caused by pathophysiologic disorders, exogenous asthma caused by environmental factors, essential asthma of unknown or unapparent origin, non-atopic asthma, bronchitic asthma, emphysematous asthma, stress-induced asthma, occupational asthma, infectious-allergic asthma caused by bacterial, fungous, protozoal or viral infection, non-allergic asthma, incipient asthma, “wheezy infant syndrome”;
chronic or acute bronchoconstriction, chronic bronchitis, obstruction of the small respiratory tract, and emphysema;
any type of obstructive or inflammatory diseases of the respiratory tract, etiology or pathogenesis, or obstructive or inflammatory diseases of the respiratory tract from the group of asthma; pneumoconiosis, chronic eosinophilic pneumonia; chronic obstructive pulmonary disease (COPD); COPD including chronic bronchitis, pulmonary emphysema or associated dyspnoea, COPD characterized by irreversible, progressive obstruction of the respiratory tract, shock lung (adult respiratory distress syndrome, ARDS), as well as aggravation of respiratory tract hypersensitivity due to therapy with other medical drugs;
pneumoconiosis of any type, etiology or pathogenesis, or pneumoconiosis from the group of aluminosis or aluminum pneumoconiosis, anthracosis (asthma), asbestosis or asbestos pneumoconiosis, chalicosis or lime pneumoconiosis, ptilosis caused by inhalation of ostrich feather dust, siderosis caused by inhalation of iron particles, silicosis or Potters asthma, byssinosis or cotton pneumoconiosis, as well as talc dust pneumoconiosis;
bronchitis of any type, etiology or pathogenesis, or bronchitis from the group of acute bronchitis, acute laryngotracheal bronchitis, bronchitis induced by peanuts, bronchial catarrh, croupous bronchitis, unproductive bronchitis, infectious asthma bronchitis, bronchitis with sputum, staphylococcal or streptococcal bronchitis; as well as vesicular bronchitis;
bronchiectasia of any type, etiology or pathogenesis, or bronchiectasia from the group of cylindrical bronchiectasia, saccular bronchiectasia, spindle bronchiectasia, bronchiole dilatation, cystic bronchiectasia, unproductive bronchiectasia, as well as follicular bronchiectasia;
seasonal allergic rhinitis, perennial allergic rhinitis, or sinusitis of any type, etiology or pathogenesis, or sinusitis from the group of purulent or non-purulent sinusitis, acute or chronic sinusitis, ethmoiditis, frontal sinusitis, maxillary sinusitis, or sphenoiditis;
rheumatoid arthritis of any type, etiology or pathogenesis, or rheumatoid arthritis from the group of acute arthritis, acute gouty arthritis, primary chronic polyarthritis, osteoarthrosis, infectious arthritis, Lyme arthritis, progredient arthritis, psoriatic arthritis, as well as spondylarthritis;
gout as well as fever associated with inflammation, or pain associated with inflammation;
eosinophile-related pathologic disorders of any type, etiology or pathogenesis, or eosinophile-related pathologic disorders from the group of eosinophilia, eosinophilic pulmonary infiltrate, Löffler's syndrome, chronic eosinophilic pneumonia, tropical pulmonary eosinophilia, bronchopneumonic aspergillosis, aspergilloma, eosinophilic granuloma, allergic granulomatous angiitis or Churg-Strauss syndrome, polyarteritis nodosa (PAN), as well as systemic necrotizing vasculitis;
atopic dermatitis, allergic dermatitis, or allergic or atopic eczema;
urticaria of any type, etiology or pathogenesis, or urticaria from the group of immune-related urticaria, complement-related urticaria, urticaria induced by material causing urticaria, urticaria induced by physical stimuli, urticaria induced by stress, idiopathic urticaria, acute urticaria, chronic urticaria, angioneurotic edema, Urticaria cholinergica, cold urticaria in its autosomal-dominant or acquired form, contact urticaria, Urticaria giantean as well as papuloid urticaria;
conjunctivitis of any type, etiology or pathogenesis, or conjunctivitis from the group of actinic conjunctivitis, acute catarrhal conjunctivitis, acute contagious conjunctivitis, allergic conjunctivitis, atopic conjunctivitis, chronic catarrhal conjunctivitis, purulent conjunctivitis, as well as spring conjunctivitis;
uveitis of any type, etiology or pathogenesis, or uveitis from the group of inflammation of the whole uvea or a part thereof, Uveitis anterior, iritis, cyclitis, iridocyclitis, granulomatous uveitis, non-granulomatous uveitis, phacoantigenic uveitis, Uveitis posterior, choroiditis, as well as chorioretinitis; psoriasis;
multiple sclerosis of any type, etiology or pathogenesis, or multiple sclerosis from the group of primary progredient multiple sclerosis, as well as multiple sclerosis with episodic course and tendency of remission;
autoimmune/inflammatory diseases of any type, etiology or pathogenesis, or autoimmune/inflammatory diseases from the group of autoimmune-hematological disorders, hemolytic anemia, aplastic anemia, aregenerative anemia, idiopathic thrombocytopenic purpura, systemic lupus erythematosus, polychondritis, scleroderma, Wegeners granulomatosis, photopathy, chronically active hepatitis, Myasthenia gravis, Stevens-Johnson syndrome, idiopathic sprue, autoimmune irritable colon disease, ulcerous colitis, Crohn's disease, endocrine opthalmopathy, Basedow's disease, sarcoidosis, alveolitis, chronic hypersensitive pneumonitis, primary biliary cirrhosis, insulin deficiency diabetes or type 1 pancreatic mellitus, Uveitis anterior, granulomatous uveitis or Uveitis posterior, dry keratoconjunctivitis, epidemic keratoconjunctivitis (diffuse), interstitial pulmonary fibrosis, pulmonary cirrhosis, mucoviscidosis, psoriatic arthritis, glomerulonephritis with and without nephrosis, acute glomerulonephritis, idiopathic nephrosis, minimal-change nephropathy, inflammatory/hyperproliferative dermal diseases, psoriasis, atopic dermatitis, contact dermatitis, allergic contact dermatitis, familial benign pemphigus, Pemphigus erythematosus, Pemphigus foliaceus as well as Pemphigus vulgaris;
preventing allograft rejection after organ transplantation,
irritable intestine (inflammatory bowel disease, IBD) of any type, etiology or pathogenesis, or irritable intestine from the group of ulcerous colitis (UC), collagenous colitis, Colitis polyposa, transmural colitis, as well as Crohn's disease (CD);
septic shock of any type, etiology or pathogenesis, or septic shock from the group of renal failure, acute renal failure, cachexia, malaria cachexia, hypophyseal cachexia, uremic cachexia, cardiac cachexia, Cachexia suprarenalis or Addison's disease, carcinomatous cachexia, as well as cachexia due to infection with human immunodeficiency virus (HIV);
liver damage;
pulmonary hypertension, as well as pulmonary hypertension induced by oxygen deficiency;
bone rarefaction diseases, primary osteoporosis and secondary osteoporosis;
any type of pathologic disorders of the central nervous system, etiology or pathogenesis, or pathologic disorders of the central nervous system from the group of depression, Parkinson's disease, learning and memory disorders, tardive dyskinesia, drug addiction, arteriosclerotic dementia, as well as dementia as an accompanying symptom of Huntington's disease, Wilson's disease, agitated paralysis, as well as thalamus atrophy;
infections, especially viral infections, such viruses increasing the production of TNF-α in their host or being sensitive to TNF-α upregulation in their host, thereby impairing their replication or other important activities, including viruses from the group of HIV-1, HIV-2 and HIV-3, cytomegalovirus, CMV; influenza, adenovirus and herpes viruses, including Herpes zoster and Herpes simplex; yeast and fungous infections, such yeasts and fungi being sensitive to upregulation by TNF-α or inducing TNF-α production in their host, preferably fungous meningitis, especially in case of simultaneous administration with other drugs of choice for the treatment of systemic yeast and fungous infections, including polymycins, preferably polymycin B, imidazoles, preferablyclotrimazol, econazol, miconazol and/or ketoconazol, triazoles, preferablyfluconazol and/or itranazol, as well as amphotericins, preferably amphotericin B and/or liposomal amphotericin B.

26. The pharmaceutical agent according to claim 13 wherein the agent is in form of a gel, poudrage, powder, tablet, sustained-release tablet, premix, emulsion, brew-up formulation, drops, concentrate, granulate, syrup, pellet, bolus, capsule, aerosol, spray and/or inhalant.

27. The pharmaceutical agent according to claim 13 present in a preparation at a concentration of from 0.1 to 99.5, preferably from 0.5 to 95.0, more preferably from 20.0 to 80.0 wt.-%.

28. The preparation of claim 27, wherein the preparation is applied orally, intravenously, intramuscularly, intraperitoneally vaginally, rectally, nasally and/or topically.

29. The pharmaceutical agent according to claim 13, wherein the agent is employed in a total amount of 0.05 to 500 mg/kg, preferably 5 to 100 mg/kg body weight per 24 hours.

30. (canceled)

31. (canceled)

32. Method for production of a medicament comprising the decoupler or a pharmaceutical agent comprising the decoupler for the prophylaxis or treatment of asthma, hypertonia, coronary heart diseases, hypertrophy of the heart, duodenal ulcer, heart failure, hepatic cirrhosis, schizophrenia, AIDS, pancreatic diabetes, insipid diabetes, obesity, chronic obstructive pulmonary diseases, and/or edemas.

33. The pharmaceutical agent of claim 13, wherein said agent is an aquaretic, contraceptive, anti-infectious, anxiolytic and/or anti-tumor agents.

34. An organism comprising the decoupler according to claim 1 and/or the recognition molecule comprising said decoupler said recognition molecule being an antibody, a complexing agent and/or a chelating agent.

35. The organism according to claim 34,
characterized in that
the organism, preferably as a result of the presence of said recognition molecule, exhibits a disease selected from the group comprising asthma, hypertonia, hypertrophy of the heart, coronary heart diseases, duodenal ulcer, heart failure, hepatic cirrhosis, schizophrenia, AIDS, pancreatic diabetes, insipid diabetes, obesity, cancer, chronic obstructive pulmonary diseases, learning disorders and/or edemas.

36. The organism according to claim 34, wherein said organism is a model for tissue- and/or cell-specific AKAP-PKA interaction, particularly as a model of insipid diabetes, pancreatic diabetes, obesity, edemas, chronic obstructive pulmonary diseases, AIDS, schizophrenia, hepatic cirrhosis, heart failure, coronary heart diseases, hypertrophy of the heart, improvement of learning, hypertonia, duodenal ulcer and/or asthma.

37. A method of modifying, preferably inhibiting, an AKAP-PKA interaction, said method comprising the steps of:
(a) providing a decoupler according to claim 1 and/or a recognition molecule comprising said decoupler, said recognition molecule being an antibody, a complexing agent and/or a chelating agent, and
(b) contacting at least one product according to (a) with a cell, a cell culture, a tissue and/or a target organism.

38. The method according to claim 18,
characterized in that
said modification is effected on a regulatory RII subunit of PKA.

39. The method according to claim 38,
characterized in that
the RII subunits are RIIalpha and/or RIIbeta subunits.

40. The decoupler of claim 1, wherein said decoupler is a lead structures in the development of pharmaceutical agents, particularly using combined and/or structure-based drug design.

41. A method for the production of pharmaceutical agents, said method comprising the following steps:
(a) providing a decoupler according to claim 1 as lead structure,
(b) chemical modification of the lead structure, preferably by means of combined and/or structure-based drug design, thereby obtaining substances, and optionally
(c) testing the substances for their capability of influencing the AKAP-PKA interaction, and selecting suitable substances as pharmaceutical agents.

42. The method according to claim 42, comprising formulating the substance into a pharmaceutically acceptable form.
